SEQUENCE LISTING (22 sequences)

SEQ ID NO: 1 - protein, 10 aa, Unknown
  Description of Unknown Organism  A screened peptide, from a random peptide library, that binds and internalizes in a FGF receptor overproducing cell line
FVPDPYRKSR

SEQ ID NO: 2 - protein, 10 aa, Unknown
  Description of Unknown Organism  A screened peptide, from a random peptide library, that binds and internalizes in a FGF receptor overproducing cell line
CGGGPVAQRC

SEQ ID NO: 3 - protein, 10 aa, Unknown
  Description of Unknown Organism  A screened peptide, from a random peptide library, that binds and internalizes in a FGF receptor overproducing cell line
CLAHPHGQRC

SEQ ID NO: 4 - protein, 4 aa, Unknown
  Consensus nuclear localization sequence
KXXX

SEQ ID NO: 5 - DNA, 47 bp, Artificial Sequence
  PCR Primer
aaaggatccgggttcccgcgtgggcgatggttgttgtcattgtcggc

SEQ ID NO: 6 - DNA, 25 bp, Artificial Sequence
  PCR Primer
ccgtaacactgagtttcgtcaccag

SEQ ID NO: 7 - DNA, 30 bp, Artificial Sequence
  PCR Primer
gcgtgggcgatggttgttgtcattgtcggc

SEQ ID NO: 8 - DNA, 25 bp, Artificial Sequence
  PCR Primer
ccacagacaaccctcatagttagcg

SEQ ID NO: 9 - DNA, 20 bp, Artificial Sequence
  PCR Primer
ttttggagattttcaacgtg

SEQ ID NO: 10 - DNA, 20 bp, Artificial Sequence
  PCR Primer
tgctaaacaactttcaacag

SEQ ID NO: 11 - protein, 4 aa, Unknown
  Lysosomal directing sequence
KCPL

SEQ ID NO: 12 - protein, 10 aa, Unknown
  Lysosomal directing sequence
DSWVEFIELD

SEQ ID NO: 13 - protein, 6 aa, Unknown
  Lysosomal directing sequence
DQRDLI

SEQ ID NO: 14 - protein, 6 aa, Unknown
  Lysosomal directing sequence
EQLPML

SEQ ID NO: 15 - protein, 26 aa, Artificial Sequence
  Endosomal escape peptide adapted from the INF7 N-terminal sequence of the influenza virus X-31 hemagglutinin subunit HA-2.
MAEGLFEAIEGFIENGWEGMIDGWYG

SEQ ID NO: 16 - DNA, 237 bp, Artificial Sequence
  Dual display vector
gaattcatgaaatacctattgcctacggccgcagcaggtctcctcctcttagcagcacaaccagcaatggccgcggagtgactcgaggatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataagaattc

SEQ ID NO: 17 - protein, 72 aa, Artificial Sequence
  Protein translation of dual display vector
MKYLLPTAAAGLLLLAAQPAMAAELEDPAKAAFNSLQASATEYIGYAWAMVVVIVGATIGIKLFKKFTSKAS

SEQ ID NO: 18 - protein, 6 aa, Unknown
  Heparin binding consensus sequence
RRXRRX

SEQ ID NO: 19 - DNA, 44 bp, Artificial Sequence
  PCR primer
ggctcgagcggccgctgcagctcacccagaaacgctggtgaaag

SEQ ID NO: 20 - DNA, 50 bp, Artificial Sequence
  PCR primer
ggctcgagggccggccggcgcgcccgccatggtgctatggctatagaacg

SEQ ID NO: 21 - protein, 14 aa, Artificial Sequence
  random peptides
CXXXXXXXXCGGGS

SEQ ID NO: 22 - protein, 10 aa, Artificial Sequence
  selected random peptide
CQQESGKQSC

(19) United States
(12) Patent Application Publication
(10) Pub. No.: US 2003/0148263 A1
(43) Pub. Date: Aug. 7, 2003

(54) Methods and compositions using genetic package display

(75) Inventors: David Larocca, Encinitas, CA (US); Paul Kassner, San Mateo, CA (US); Andrew Baird, London (GB); Michael Alan Burg, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Selective Genetics, Inc., San Diego, CA (US)

(21) Appl. No.: 10/151,204

(22) Filed: May 17, 2002

Related U.S. Application Data
(63) Continuation-in-part of application No. 09/866,073, filed on May 24, 2001.
Application No. 09/866,073 is a continuation-in-part of application No. PCT/US99/25361, filed on Oct. 29, 1999.
Application No. PCT/US99/25361 is a continuation-in-part of application No. 09/258,689, filed on Feb. 26, 1999, now Pat. No. 6,451,527.
Application No. 09/258,689 is a continuation-in-part of application No. 09/193,445, filed on Nov. 17, 1998.
Application No. 09/258,689 is a continuation-in-part of application No. 09/195,379, filed on Nov. 17, 1998, now Pat. No. 6,472,146.
(60) Provisional application No. 60/057,067, filed on Aug. 29, 1997.

(51) Int. Cl. C12Q 1/70; C12Q 1/68; C12N 15/74

(52) U.S. Cl. 435/5; 435/6; 435/320.1

(57) ABSTRACT

A genetic package display system and methodology for probing protein-protein interactions that lead to cell transduction, selecting and/or identifying internalizing ligands, target cells and tissues which internalize known or putative ligands, and cell transduction facilitating peptides is provided. A ligand displaying genetic package that carries a selectable marker (e.g., reporter, selection, etc.) and presents a ligand on its surface is utilized to identify internalizing ligands, tranduction facilitating peptides, and/or a variety of cells and tissue types for the ability to be successfully transduced by the ligand displaying genetic package. Also provided are methods for evolving a ligand displaying package to facilitate gene delivery or delivery of any desired agent (e.g., pharmaceutical, polypeptide, peptide, etc.) into a cell and/or targeting cellular compartments such as the nucleus, endosome, chloroplast, mitochondria, etc.

### CROSS REFERENCE TO RELATED APPLICATIONS

The present application is a continuation-in-part application of U.S. application Ser. No. 09/866,073, filed May 24, 2001, which in turn is a continuation-in-part application of PCT Application No. PCT/US99/25361, published May 25, 2000 and filed Oct. 29, 1999, which in turn claims priority to U.S. application Ser. No. 09/258,689, filed Feb. 26, 1999, which in turn is a continuation-in-part application of U.S. application Ser. Nos. 09/193,445 and 09/195,379, both filed Nov. 17, 1998, which in turn are continuation-in-part applications of U.S. application Ser. No. 09/141,631, filed Aug. 28, 1998, which in turn claims priority to U.S. Provisional Application Ser. No. 60/057,067, filed Aug. 29, 1997, now abandoned.

### BACKGROUND OF THE INVENTION

1. Field of the Invention

This invention relates generally to genetic package display (e.g., phage display), and in particular, to selection of ligands that bind to a cell surface receptor/moeity and internalize useful in delivery of molecules to the nucleus of a target cell and for use in gene therapy, screening, and in various methodologies. The methods described herein are also referred to as Ligand Identification Via Expression™ or LIVE™.

2. Description of the Related Art

Bacteriophage expressing a peptide on its surface has been used to identify protein binding domains including antigenic determinants, antibodies that are specifically reactive, mutants with high affinity binding, novel ligands, and substrate sites for enzymes. In its most common form, a peptide is expressed as a fusion protein with a coat protein of a filamentous phage. This results in the display of the foreign protein on the surface of the phage particle. Libraries of phage are generated that express a multitude of foreign proteins. These libraries are bound to a substrate or cell that presents the binding partner of interest. This screening process is essentially affinity purification. Bound phage are recovered, propagated, and the genes encoding the foreign proteins may be isolated and characterized. This technology is commonly referred to as “phage display.”

Through a process called “biopanning,” the specific phage carrying a peptide or protein that interacts with a protein or other moiety on a solid phase can be identified and isolated. However, in many applications, binding or binding affinity is not the sole critical parameter. One example is applications wherein intracellular delivery is desirous, such as gene therapy. A ligand that is capable of being internalized in its native form, when used in a system for intracellular delivery, may not be efficiently internalized or while internalized may not lead to gene expression. Further, many ligands that are found to internalize do not facilitate tranduction of the targeted cells, leaving the internalized nucleic acid sequences in a non-functional state.

Phage libraries can be screened for potentially internalizing ligands by biopanning on live cells and rescuing internalized phage from the cells after stripping off externally bound phage (e.g., acid elution). However, this method may result in recovery of undesired phage that bind very tightly or are only partially internalized. Moreover, phage that are internalized and subjected to proteases lose infectivity and can not be recovered. Molenaar et al., Virology 293:182-191, 2002.

Generally speaking, the selection of ligands from phage display libraries or other genetic packages relies on peptide affinity and avidity. The number of phage recovered is determined by the complexity of the library, target protein, and selection stringency. Accordingly, prior to the present invention, three types of selection have been evaluated over the last several years: 1) affinity selection against simple targets like immobilized proteins; 2) affinity selection against complex targets such as cell surfaces; and 3) selection through phage processing such as their internalization by cells.

When utilizing affinity selection, unbound phage are washed away with buffers of different stringencies, and the remaining attached phage particles are recovered, amplified in bacteria, and then further enriched by repeated rounds of adsorption and recovery. In early rounds of the selection, a specific binding phage may be present among millions, if not billions, of other non-specific phage particles, depending on the complexity of the library. Moreover, while the phage recovered may be present in extremely low concentration, they must be recovered in an infective form in order to allow for amplification by infecting host bacteria. As the round of selection is repeated, the library is significantly reduced in complexity and phage encoding the binding ligands can then be characterized by DNA sequencing.

However, the ability to recover the most useful cell binding ligands rapidly is extremely limited. In theory, if it were possible to incubate a phage library with a target for sufficient time with efficient washing away of non-binders, while leaving binders intact such that they can be recovered as infective phage, then one would be able to isolate a relatively pure population of binding phage in a single round of selection. However, this is rarely the case, and even the relatively simple selection of binding phage against a purified molecular target requires several rounds to “affinity purify” the desired phage. Selection against more complex targets, such as whole cells, can take six or more rounds of selection, and this is only for detecting ligands that bind, excluding internalization. There are several possible explanations for why several rounds of screening are necessary, one being the problem of background. Even so-called non-binders have a certain affinity for the target (or the solid support, cell surface, etc.). Since non-binders are commonly in million fold or more excess, even low affinity binders will result in a background of false positives. On the other hand, true binders of modest affinity may be lost if washes are too stringent, and tight binders could be lost if they can not be removed from the target or if removal results in loss of infectivity.

When selecting phage libraries against a complex target, the hurdles are even more substantial, since the target as well as the binding phage are both present in low concentrations. Further, as disclosed in the present application, one can take advantage of the cell's ability to internalize functional ligands as a way to capture phage displaying receptor binding ligands. In this case, the off rate of the reaction goes to infinity since the binding phage is never released. Acid washing or protease treatment can be used to remove phage that stick non-specifically to the cell surface to reduce background. The problem then becomes how to recover the internalized phage. In traditional biopanning for internalized phage, the cells are lysed, the lysate is contacted with a suitable host bacteria, and new phage are prepared from the infected bacteria. It has been demonstrated, however, that internalized phage lose infectivity as they pass through the endosome to the lysosome, since chloroquine can be used to prevent this loss to some degree. Ivanenkov et al., Biochimica et Biophysica Acta 1448:450-462, 1999 and Ivanenkov et al., Biochem Biophys Res Commun 276(1):251-7, 2000. However, in practice three to eight rounds of selection are required to identify internalizing peptides from a phage display library. Even when screening is complete, it is not known if rare binders are lost in the early rounds. Thus, a more sensitive way of recovering internalized phage would be useful for both shortening the number of selection rounds and for recovering a higher percentage of the complete repertoire of binding phage present in the original library. Moreover, even more complex libaries (>10⁹ members) could be screened, if the selection for internalized phage could be made more sensitive while keeping the background low. The present invention fulfills such a need.

Ligand selection using phage libraries screened against whole cells has been investigated. See, Hoogenboom et al., European J. of Biochem. 260:774-784, 1999; Szardenings et al., J. Biol. Chem. 272:27943-27948, 1997; Pereira et al., J. Immunol Meth 203:11-24, 1997; Pasqualini et al., Nature 380:364-366, 1996. An apparent advantage of this kind of “biopanning” using whole cells is that little or no prior knowledge of a target molecule (e.g., a receptor) is needed, and the target molecule can be in its native form on the cell surface. However, a target protein may be low in concentration relative to other cell surface proteins. This presents a significant disadvantage to selection and, as in the affinity selection against immobilized targets, non-specifically adherent phage can give false positive signals. Similarly, a low concentration of non-specific phage can interfere in the early rounds of selection when the specific binding phage are in extremely low concentrations.

Recent studies have demonstrated that vasculature-specific organ homing peptides can be selected from libraries that are “biopanned” in vivo. For instance, by applying standard phage display selection techniques to mice, in vivo, Pasqualini et al. identified peptides that are capable of selectively targeting phage to the vasculature of different organs including, brain, prostate, and kidney. Pasqualini and Ruoslahti, Mol. Psychiatry 1(6):423, 1996. However, Pasqualini et al. did not select phage that target parenchymal cells of tissues, but rather only the endothelial cells of the vasculature of these tissues. See, e.g., Molenaar et al., Virology 293:182-191, 2002.

In an effort to increase selection stringency and overcome the problems of non-specific adsorption that are associated with biopanning against whole cells, alternative strategies have been explored. For instance, Hart et al. initially demonstrated that RGD targeted phage were internalized through receptor mediated endocytosis. J. Biol. Chem. 269(17):12468-12474, 1994. Subsequently, Barry et al. showed that cell-specific internalizing peptides could be selected from large diverse libraries of displayed peptides by washing phage off the cell surface at low pH and recovering internalized phage from cell lysates. Nat. Med. 2:299-305, 1996. However, these methods suffered from the requirement of multiple steps as well as no clear ability to determine in an initial screen which ligands would facilitate gene transduction and which would not. The original rationale behind selection by internalization was merely to increase the stringency of selection and therefore increase the ratio of signal to background.

Accordingly, current methodologies are inadequate to determine the usefulness of ligands for facilitating transfer and transduction of a cell by a nucleic acid molecule associated with the genetic package and ligand.

Further, identification of target cells or tissues that are able to internalize ligands and express a transgene would readily allow one to identify specific target cells for known or putative ligands, as well as allow one to identify ligands for specific cell or tissue types. However, current methods of target cell identification are hampered by the same difficulties, as noted above, with regard to screening for internalizing ligands. Accordingly, current methodologies are inadequate to determine which cell or tissue types are useful targets for ligand mediated gene transfer.

Thus, current screening methods are inadequate for selecting peptide or protein ligands that bind to a cell surface receptor, internalize, and lead to expression of the carried nucleic acid molecule. The present invention discloses display methods that select peptide or protein ligands that internalize and facilitate cell transduction and expression of product from an associated nucleic acid molecule, and further provides other related advantages. In addition, the present invention provides the ability to perform in-vivo selection of ligands that target and internalize in specific tissues, rather than simply the vasculature of selected tissues.

The present invention further provides novel vectors useful for practicing the methods of the invention. In one embodiment, the invention provides a novel cloning vector for selecting in-frame libraries, which may be used according to the present invention to produce vectors useful for practicing screening methods of the present invention. In another related embodiment, the invention provides a retro-vector useful for selecting internalizing/transducing phage display ligands capable of nuclear localization and gene expression, according to methods of the invention.

### BRIEF SUMMARY OF THE INVENTION

The present invention utilizes novel genetic package display of putative ligands to investigate the ability of these molecules to facilate cellular transduction with associated nucleic acid molecules, while also providing a functional genomic benefit, in that a variety of sequences can be screened for their ability to successfully deliver a reporter gene or other nucleic acid molecule to a cell by analyzing expression of that nucleic acid molecule or an expressed product. The finding that this could be achieved is quite surprising, as many genetic packages, including filamentous phage, were thought to be too large to pass through an endosomal pathway, and, even if they did, it seemed unlikely that a bacterial virus could traffic appropriately through the endosomal environment, uncoat, and express their single-stranded DNA in a mammalian cell. Barry et al. (supra); Molenaar et al. (supra). Remarkably, however, as demonstrated herein, significant levels of gene transfer are obtained when phage are targeted to mammalian cells.

The present invention also provides additional embodiments including vector designs for selecting in-frame inserts and methods of using such vectors. In addition, the present invention provides additional screening methods utilizing PCR and alternative DNA amplification methods, such as rolling circle amplification or the like, for example. The invention also provides a novel method of detecting internalized ligand displaying genetic packages that are expressed in a host or target cell through the use of a reporter containing an intron, such that the detectable product is expressed following mRNA expression in a mammalian cell.

In one embodiment of the invention, the invention provides a vector for selecting in-frame inserts. The vector contains a nucleic acid sequence encoding a coat protein signal sequence and another nucleic acid sequence encoding a selectable marker, wherein the in-frame insertion of an insert nucleic acid sequences allows expression of the selectable marker. In a specific embodiment, the vector has one insert.

In one embodiment, the above vector also contains an inducible promoter. In a specific embodiment, the inducible promoter is araC.

In certain embodiments, the coat protein signal sequence is from a bacteriophage. In specific embodiments, the bacteriophage coat protein is M13 gIII, M13 gVI, M13 gVII, M13 gVIII, M13 gIX, fd minor coat protein pIII, lambda D protein, lambda phage tail protein pV, fr coat protein, Ø29 tail protein gp9, MS2 coat protein, T4 small outer capsid protein, T4 nonessential capsid scaffold protein IPIII, T4 lengthened fibritin protein gene, PRD-1 gene III, Qβ3 capsid protein, or P22 tailspike protein.

In another embodiment of the invention, the selectable marker of a vector of the invention is an antibiotic resistance gene. In one specific embodiment, the antibiotic resistance gene is an ampicillin resistance gene.

In a related aspect, the invention includes a method for making a library of vectors with in-frame inserts by introducing a mixture of nucleic acid inserts into a vector of the invention, resulting in insert-containing vectors; transducing the insert-containing vectors into bacteria; and selecting bacterial clones that grow under selective conditions specific for the selectable marker. In one embodiment, this method includes recovering the vectors from the bacterial clones. In one embodiment, the method includes isolating the inserts from the vectors and inserting the isolated inserts into a phage genome or a phagemid. In one embodiment of the invention, the phagemid is a pUC-based phagemid. In specific embodiments, the pUC-based phagemid is pUC198, pUC207, or pUC250. In another embodiment, the phage genome or phagemid comprises a marker gene capable of being expressed in a mammalian cell.

In certain embodiments, the mixture of nucleic acid inserts is derived from a cDNA library. In specific embodiments, the mixture is derived from an antibody gene library, a peptide gene library, or a mutein library.

In a related embodiment, the invention includes a library of vectors with in-frame inserts produced by a method of the invention. In one embodiment, the vectors are pUC-based phagemids. In specific embodiments, they are pUC198, pUC207, or pUC250.

In principle, the genetic selection of functional ligands by the Ligand Identification Via Expression (LIVE) methods set forth herein represents a significant departure from traditional biopanning (see Table 1), because it increases the stringency of selection by requiring the displayed ligand to bind, internalize, traffick to the desired cellular location, and deliver a selectable genetic marker. This increased stringency decreases background as compared to phage displaying simple binding proteins. In addition, biopanning relies on the recovery of infective phage, whereas selection by the methods described herein does not require the presence of infective phage. Therefore, the present methods allow one to recover phage that are subjected to proteolytic cleavage after internalization and would otherwise be lost during biopanning. Moreover, because certain selection methods used in the present invention are genetic, a stable inherited change in the cell (e.g., marker expression) can be used as the basis for selection. Thus, for example, it is feasible that stable cell colonies could be used to directly identify rare phage internalization events in one round of screening.

TABLE 1
Comparison of phage selection on cells using Biopanning versus LIVE ™.

| Biopanning and/or Internalization | |
|---|---|
| Screening | LIVE ™ |
| Selects by affinity or internalization | Selects by internalization |
| | and gene transfer |
| Requires recovery of infective phage | Does not require infective |
| | phage for identification |
| | and further analysis |
| High background from adherent phage | Low background from |
| | adherent phage |
| Selection transitory | Can select cells having |
| | stable genetic change |
| | (e.g., marker expression/ |
| | drug resistance) |
| Efficiently internalized phage lost during | Most efficiently |
| panning procedure | internalized phage having |
| | gene transfer |
| | ability selected |

The present invention also offers other unexpected advantages over biopanning. A direct comparison of the ligands identified by LIVE versus biopanning revealed two surprising results. First, the LIVE method identified more different ligands than biopanning (data not shown). In addition, the LIVE method identified ligands not identified using biopanning, while biopanning failed to identify a ligands not identified by the LIVE method (data not shown). Thus, the LIVE method allows the identification of a more diverse pool of ligands, as compared to biopanning.

Accordingly, through the use of the present invention one of ordinary skill in the art could functionally assess a variety of displayed peptides, polypeptides, etc. for the ability to facilitate internalization and genetic transduction. Thus, a logical extension of this methodology is the use of these methods to functionally explore the existence of natural ligands present in existing libraries, such as those now deposited due to the human genome project. In this case, it is the cell surface, itself, that selects the “most fit” ligands based upon their ability to stimulate receptor mediated endocytosis and subsequent phage transduction. The identification of novel ligands and their receptors using the present methodologies is likely to lead to new drugs and drug targets, because cell-surface interacting ligands effect critical cellular processes like cell growth and differentiation. After all, natural ligands having direct clinical utility are the leading therapeutic products in biotechnology (e.g., erythropoietin, growth hormone, IL-2, and GM-CSF). Yet, such ligands are the most difficult to mine from published genomic databases, because they often exist as small fragments contained in much larger genes that are processed in a cell specific fashion.

Further, the present invention lends itself to the discovery of ligands useful for more traditional therapeutics. For example, once a sequence is identified that facilitates genetic transduction, this ligand could be used to target small molecules (e.g., pharmaceutical drugs) to the nucleus of a cell or to other “targeted” areas within a cell, thus increasing the therapeutic efficacy of the associated drug.

Within one aspect of the present invention, a method of selecting internalizing ligands displayed on a genetic package is presented, comprising: (a) contacting a ligand displaying genetic package(s) within a cell(s), wherein the package carries a gene encoding a detectable product which is expressed upon internalization of the package; and (b) detecting product expressed by the cell(s); thereby selecting internalizing ligands displayed on a genetic package.

In another aspect, the invention provides a method of identifying an internalizing ligand displayed on a genetic package, comprising: (a) contacting one or more ligand displaying genetic packages with a cell(s), wherein each package carries a gene encoding a selectable marker which is expressed upon internalization of the package, (b) detecting the selectable marker expressed by the cell(s); and (c) recovering a nucleic acid molecule encoding an internalizing ligand from the cell(s) expressing the product, and thereby identifying an internalizing ligand displayed on a genetic package.

In yet another aspect, the invention provides a method of identifying an internalizing ligand displayed on a genetic package, comprising: (a) contacting one or more ligand displaying genetic packages with a cell(s), wherein each package carries a gene encoding a selectable product which is expressed upon internalization of the package, (b) incubating the cell(s) under selective conditions; and (c) recovering a nucleic acid molecule encoding an internalizing ligand from the cell(s) which grow under the selective conditions; thereby identifying an internalizing ligand displayed on a genetic package.

In yet another aspect, a method is provided for a high throughput method of identifying an internalizing ligand displayed on a genetic package, comprising: (a) contacting one or more ligand displaying genetic packages with a cell(s) in an array, wherein each package carries a gene encoding at least one detectable product which is expressed upon internalization of the package; and (b) detecting product(s) expressed by the cell(s) in the array, and thereby identifying an internalizing ligand displayed on a genetic package. In one embodiment, the ligand displaying package comprises a library of ligand displaying packages.

In another aspect, the present invention provides a method of identifying an internalizing ligand displayed on a genetic package, comprising: (a) contacting one or more ligand displaying a genetic packages with a cell(s), wherein each package carries a selectable marker which is detectable upon internalization of the package, (b) detecting the selectable marker internalized by the cells; and (c) recovering a nucleic acid molecule encoding an internalizing ligand from the cell(s) carrying the selectable marker, thereby identifying an internalizing ligand displayed on a genetic package.

Within one aspect of the present invention, a method of selecting internalizing ligand/anti-ligand pairs is presented, comprising: (a) contacting a ligand displaying genetic package(s) with a cell(s), wherein the package carries a gene encoding a detectable product which is expressed upon internalization of the package; and (b) detecting product expressed by the cell(s); thereby selecting ligand/anti-ligand pairs.

In another aspect, the invention provides a method of identifying a ligand or anti-ligand of an internalizing ligand/anti-ligand pair, comprising: (a) contacting one or more ligand displaying genetic packages with a cell(s), wherein each package carries a gene encoding a detectable product which is expressed upon internalization of the package, and wherein the cell(s) expresses an anti-ligand-receptor fusion protein on its surface; (b) detecting product expressed by the cell(s); and (c) recovering a nucleic acid molecule encoding an internalizing ligand and/or a nucleic acid molecule encoding an internalizing anti-ligand from the cell(s) expressing the product, and thereby identifying a ligand or anti-ligand of a internalizing ligand/anti-ligand pair.

In yet another aspect, the invention provides a method of identifying a ligand or anti-ligand of an internalizing ligand/anti-ligand pair, comprising: (a) contacting one or more ligand displaying genetic packages with a cell(s), wherein each package carries a gene encoding a detectable product which is expressed upon internalization of the package, and wherein the cell(s) expresses an anti-ligand-receptor fusion protein on its surface; (b) incubating the cell(s) under selective conditions; and (c) recovering a nucleic acid molecule encoding an internalizing ligand and/or a nucleic acid molecule encoding an internalizing anti-ligand from the cell(s) which grow under the selective conditions; thereby identifying a ligand or anti-ligand of a internalizing ligand/anti-ligand pair.

In yet another aspect, a method is provided for a high throughput method of identifying a ligand or anti-ligand of an internalizing ligand/anti-ligand interactions, comprising: (a) contacting one or more ligand displaying genetic packages with a cell(s) in an array, wherein each package carries a gene encoding at least one detectable product which is expressed upon internalization of the package; and (b) detecting product(s) expressed by the cell(s) in the array, and thereby identifying a ligand or anti-ligand of a internalizing ligand/anti-ligand interactions. In one embodiment, the array contains cells expressing a library of anti-ligand-receptor fusion proteins. In another embodiment, the ligand displaying package comprises a library of ligand displaying packages.

Within one aspect of the present invention, a method of identifying a target cell or tissue for internalizing ligands is presented, comprising: (a) contacting a library of ligand displaying genetic packages with a cell(s) or tissue(s), wherein each package carries a gene encoding a detectable product which is expressed upon internalization of the package; and (b) detecting product expressed by the cell(s) or tissue(s), and thereby identifying a target cell or tissue for internalizing ligands.

In another aspect, the invention provides a method of selecting an internalizing ligand for a selected target cell or tissue within a pool of target cells or tissues and identifying a target cell or tissue for the internalizing ligand, comprising: (a) contacting a library of ligand displaying genetic packages with a pool of cell(s) or tissue(s), wherein each package carries a gene encoding a selectable marker which is expressed upon internalization of the package; (b) detecting the selectable marker expressed by the cell(s) or tissue(s); and (c) recovering a nucleic acid molecule encoding an internalizing ligand from a selected set of cell(s) or tissue(s) within the pool expressing the product.

In yet another aspect, the invention provides a method of selecting an internalizing ligand for a selected target cell or tissue within a pool of target cells or tissues and identifying a target cell or tissue for the internalizing ligand, comprising: (a) contacting a library of ligand displaying genetic packages with a pool of cell(s) or tissue(s), wherein each package carries a gene encoding a detectable product which is expressed upon internalization of the package; (b) incubating the cell(s) or tissue(s) under selective conditions; and (c) recovering a nucleic acid molecule encoding an internalizing ligand from a selected set of cell(s) or tissue(s) within the pool which grow under the selective conditions; thereby selecting internalizing ligands and identifying a target cell or tissue for the internalizing ligand.

In yet another aspect, a method is provided for a high throughput method of identifying target cells or tissues for internalizing ligands, comprising: (a) contacting a library of ligand displaying genetic packages with cells or tissue in an array, wherein each package carries a gene encoding at least one detectable product which is expressed upon internalization of the package; and (b) detecting product(s) expressed by the cells or tissue in the array; thereby identifying target cells or tissues for internalizing ligands. In one embodiment, the array contains a variety of cell types. In another embodiment, the method further comprises step (c), wherein the library is a library of ligand displaying bacteriophage that is repeatedly divided into subset pools and screened using steps (a) and (b) until a specific bacteriophage expressing an internalizing ligand is identified.

In yet additional embodiments a medicament for gene therapy is provided, comprising an internalizing ligand identified by the of the present invention. Also provided are anti-bacterial agents comprising an internalizing ligand identified by the methods of the present invention.

Also provided are methods for identifying transduction facilitating peptides, comprising: (a) contacting one or more ligand displaying a genetic packages with a cell(s), wherein each package displays a putative transduction facilitating peptide and a ligand known to internalize, and wherein each package carries a selectable marker which is detectable upon internalization of the package, (b) detecting the selectable marker internalized by the cells; and (c) recovering a nucleic acid molecule encoding an internalizing ligand from the cell(s) carrying the selectable marker, and thereby identifying an internalizing ligand displayed on a genetic package.

In related embodiments, the selectable marker is selected from reporter gene expression, expression of a gene that confers the ability to permit cell growth under selection conditions, non-endogenous nucleic acid sequences that permit PCR amplification, and nucleic acid sequences that can be purified by protein/DNA binding.

In certain embodiments, the ligand displaying genetic package comprises a bacteriophage. The bacteriophage are filamentous phage or lambdoid phage in other preferred embodiments. In some embodiments, the bacteriophage carries a genome vector. In other embodiments, the bacteriophage carries a hybrid vector.

In other embodiments, the library is a cDNA library, an antibody gene library, a random peptide gene library, or a mutein library. In other preferred embodiments, the detectable product is selected from the group consisting of green fluorescent protein, β-galactosidase, secreted alkaline phosphatase, chloramphenicol acetyltransferase, luciferase, human growth hormone and neomycin phosphotransferase.

In other embodiments, the cells may be isolated by flow cytometry, for example. In further embodiments, the methods further comprise recovering a nucleic acid molecule encoding the ligand from the cell(s) expressing the product. Also provided are methods for enhancing transduction by utilizing genotoxic agents, heat shock, and transduction facilitating peptides.

In certain embodiments, PCR, RT-PCR, rolling circle amplification, or Hirt extraction methods are used to recover the internalized nucleic acid molecules. In certain embodiments, recovered nucleic acid molecules are DNA or RNA.

In one embodiment, a detectable marker comprises an intron, and the marker is detected following mRNA process in a mammalian cell.

These and other aspects of the present invention will become evident upon reference to the following detailed description and attached drawings. In addition, various references are set forth below which describe in more detail certain procedures or compositions (e.g., plasmids, etc.), and are therefore incorporated herein by reference in their entirety.

### BRIEF DESCRIPTION OF THE SEVERAL VIEWS OF THE DRAWINGS

FIGS. 1A and 1B are schematic representations of phage vectors for mammalian cell transduction. FIG. 1A depicts the parent phage vector with wild type pIII coat protein. The base vector is M13 genome with the ampicillin resistance (Amp^{R}) gene and a GFP expression cassette inserted into the intergenic region between pIV and pII (MEGFP3). The MEGFP3 vector contains the following elements: ori-CMV, SV40 replication origin and CMV promoter; EGFP, enhanced green fluorescent protein gene; BGH; and a bovine growth hormone polyadenylation sequence. FIG. 1B represents the FGF-pIII fusion display phage (MF2/1G3).

FIG. 2 is a scanned image of a Western Blot analysis representing detection of FGF2-pIII fusion protein in protein extracts from purified FGF2-phage (FGF2-MEGFP).

FIGS. 3A and 3B are bar graphs of ELISA detection of FGF2 on FGF2-phage. FIG. 3A depicts the amount of phage protein detected using both the empty MEGFP3 (i.e., MG3) vector and the FGF2 fusion construct (FGF2-MEGFP). FIG. 3B depicts the amount of FGF2 detected on the phage having the fusion construct.

FIGS. 4A and 4B are bar graphs representing the transduction of COS cells by FGF2-phage.

FIG. 5 is a bar graph representing the transduction of COS cells by peptide display phage.

FIG. 6 is a scanned image of a Western Blot analysis representing detection of EGF-pIII fusion protein in protein extracts from purified EGF-phage.

FIG. 7 is a bar graph representing the dose response of COS cells to various phage titers.

FIG. 8 is a bar graph representing a time course analysis of various incubation times and the effect on transduction.

FIGS. 9A and 9B are bar graphs representing the specificity of transduction of COS cells by EGF-phage.

FIG. 10 is a bar graph representing transduction specificity of a variety of human carcinoma cells.

FIG. 11 is a scanned image of ethidium bromide stained gel electrophoretic analysis of products obtained by PCR amplification of pIII genes/pIII gene fusions following various rounds of selection.

FIG. 12 is a vector map of an AAV-Phage hybrid genome vector.

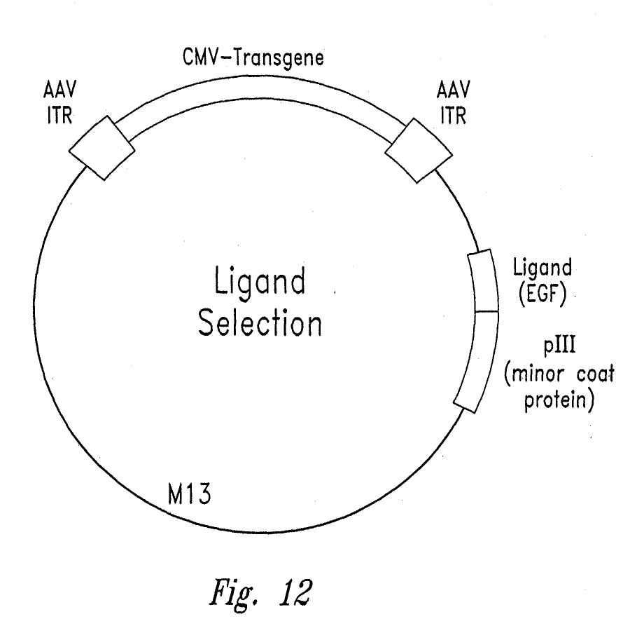

FIG. 13 is a vector map of an AAV-Phage hybrid phagemid vector.

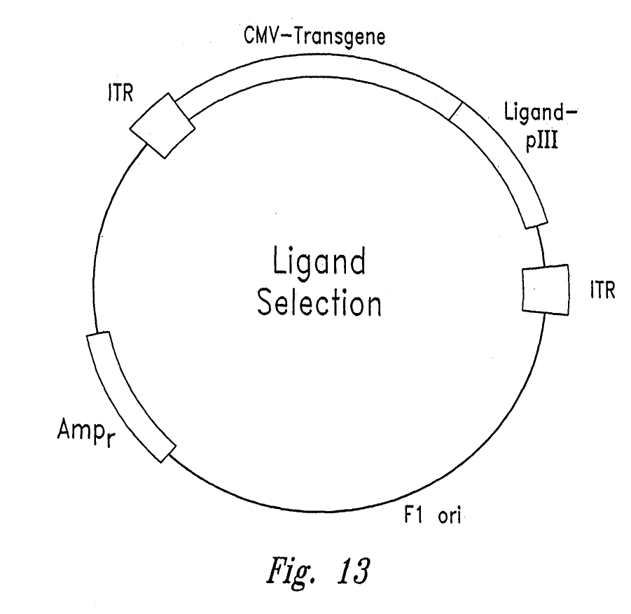

FIG. 14 is a bar graph depicting the effects on transduction following genotoxic treatment and/or heat shock treatment of target cells.

FIG. 15 is a bar graph representing the effects on transduction following display of an endosomal escape peptide.

FIG. 16 is a bar graph displaying the combined effect of heat shock and display of an endosomal escape peptide on transduction.

FIG. 17 is a vector map of a pUC-MG4 phagemid vector.

FIG. 18 is a vector map of a pUC-MG4-38 dual display vector.

FIG. 19 is a graph representing the dose-dependent increase in colonies.

FIG. 20 is a graph representing the time-dependent increase in the number of colonies.

FIG. 21 is a vector map of pBADamp.

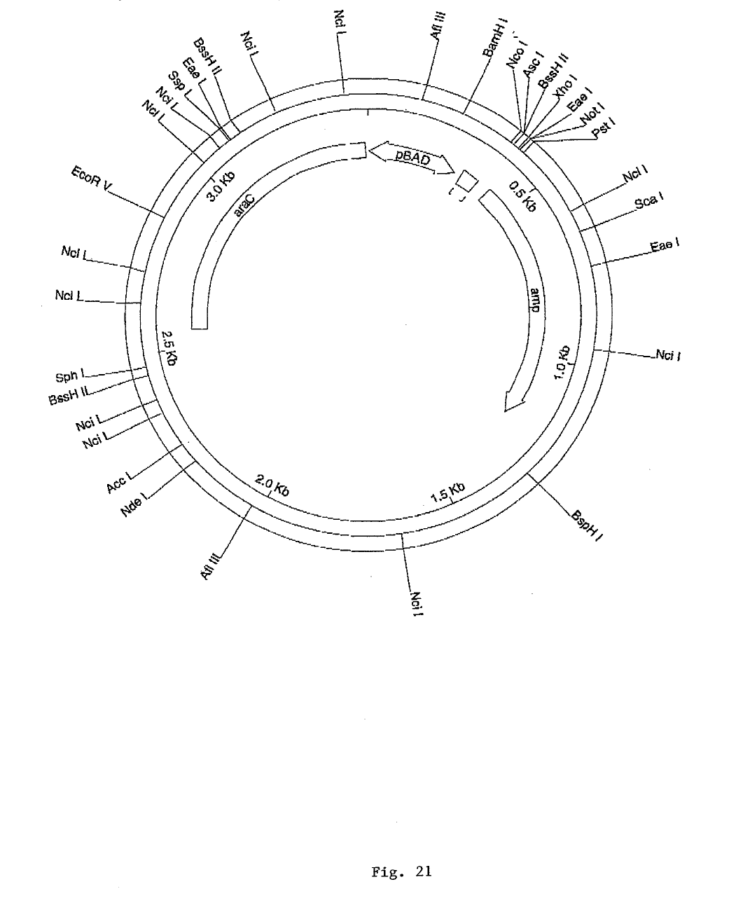

FIG. 22 is a diagram depicting comparative amplification and quantification of circular genetic packages (amplification products) containing selectable antibiotic resistance markers.

FIG. 23 is a vector map of pRV-198.

### DETAILED DESCRIPTION OF THE INVENTION

As noted above, the present invention provides methods of using ligand displaying genetic packages to identify protein-protein interactions, ligands that bind and internalize and lead to genetic transduction of a marker nucleic acid molecule, to identify target cells and/or tissues for known or putative ligands, or to identify transduction facilitating peptides. While it should be understood that a variety of ligand display genetic packages may be utilized (e.g., phage display, yeast display, RNA-peptide fusions, adenoviral display, retroviral display, and ligand displaying bacteria), the present invention uses bacteriophage ligand display to exemplify the various embodiments.

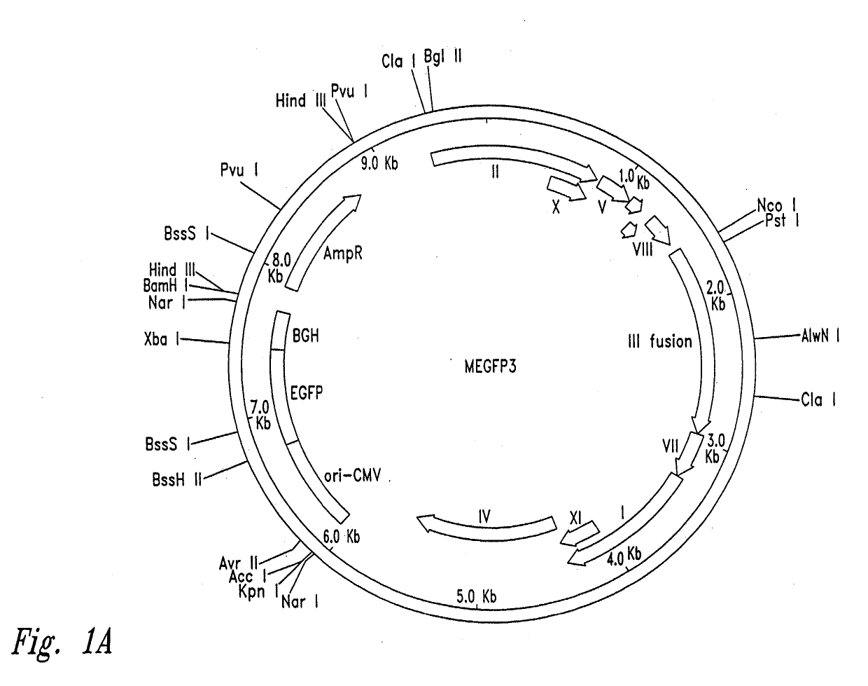

Briefly, in one aspect of the present invention, a library of antibodies, cDNAs, or genes encoding random peptides is cloned into a coat protein of a ligand displaying genetic package (e.g., gene III protein of filamentous phage). The phage genome also contains an “expression cassette” encoding a transgene/marker nucleic acid molecule placed downstream from a cell promoter that is active in the cells to be infected (FIG. 1A). The transgene is generally a selectable gene product and/or a detectable marker. Phage are contacted with test cells, and expression of the transgene is monitored or selected. Desirable genetic packages, such as phage that internalize and lead to transgene expression, will confer the phenotype of the transgene, such as drug resistance or expression of a fluorescing protein. The cells may be isolated on the basis of transgene expression. For example, when the transgene is a drug resistance gene, cells are grown in the presence of the drug, such that only those cells receiving and expressing the transgene are propagated. The gene(s) that are fused with the coat protein and promotes cell binding, internalization, and transgene expression are recovered from the selected cells by a suitable method.

In certain aspects of the present invention a variety of modifications may be utilized. For example, nucleic acid sequences may be recovered from targeted cells by Hirt extraction, by PCR, by RT-PCR of the encoded RNA transcript of the encoded detectable marker, by utilizing a binding agent such as a primer or a nucleic acid binding domain that is labeled and allows separation once bound to phage nucleic acid sequences (e.g., the primer could be biotinylated and then mixed with a streptavidin conjugated magnetic bead, thereby allowing extraction of only the sequences, such as phage sequences, bound by the primer), or by DNA amplification methods that use rolling circle methods such as phi 29 polymerase. In addition, vectors for selection of in-frame inserts may be utilized to enhance selection of only in-frame sequences from cDNA, mutein, or other nucleic acid libraries.

I. Display Packages

A variety of ligand displaying genetic packages may be used within the context of the present invention. A “ligand displaying genetic package”, as used herein, refers to any package that comprises a peptide/protein ligand and carries a nucleic acid molecule which is itself detectable, or which expresses a detectable product, once internalized in a target cell. Detectable products, therefore, as used herein, include any DNA, RNA, or polypeptide sequence capable of being detected following introduction into a cell by any method known and available in the art. In one aspect, a nucleic acid molecule carried by the ligand displaying genetic package is expressed upon internalization into a cell, thereby allowing for recovery and detection of the internalized genetic package. In other aspects, the ligand displaying genetic package may carry a nucleic acid molecule that allows for detection by a variety of methods, including RT-PCR of RNA transcripts derived from the delivered nucleic acid, PCR of unique sequences, rolling circle DNA amplification (e.g., phi 29 polymerase), the Hirt extraction method (Hirt, J. Mol. Biol. 26:365-369, 1967), and the like, or by the ability of the internalized nucleic acid sequences to bind to non-endogenous DNA binding proteins (e.g., nucleic acid sequence could comprise a lac operon, thereby allowing for lac repressor binding, binding domains, labeled primers, etc.). Accordingly, display may be by a virus, RNA-peptide fusions, bacteriophage, yeast, bacteria, or similar system (See, Phage Display of Peptides and Proteins, pages 151-193, Kay (Ed.), Academic Press, San Diego, 1996). Certain specific embodiments described herein utilize bacteriophage. Such phage include the filamentous phage, lambda, T4, MS2, and the like. A preferred phage is a filamentous phage, such as M13 or f1. Accordingly, many illustrations, while exemplifying the use of phage, could also be performed with any ligand displaying genetic package.

Phage that present a foreign protein or peptide as a fusion with a phage coat protein are designed to contain appropriate coding regions of the coat proteins. A variety of bacteriophage and coat proteins may be used. Examples include, without limitation, M13 gene III, gene VI, gene VII, gene VIII, and gene IX; fd minor coat protein pIII (Saggio et al., Gene 152:35, 1995); lambda D protein (Sternberg and Hoess, Proc. Natl. Acad. Sci. USA 92:1609, 1995; Mikawa et al., J. Mol. Biol. 262:21, 1996); lambda phage tail protein pV (Maruyama et al., Proc. Natl. Acad. Sci. USA 91:8273, 1994; U.S. Pat. No. 5,627,024); fr coat protein (WO 96/11947; DD 292928; DD 286817; DD 300652); φ29 tail protein gp9 (Lee, Virol. 69:5018, 1995); MS2 coat protein; T4 small outer capsid protein (Ren et al., Protein Sci. 5:1833, 1996), T4 nonessential capsid scaffold protein IPIII (Hong and Black, Virology 194:481, 1993), or T4 lengthened fibritin protein gene (Efimov, Virus Genes 10:173, 1995); PRD-1 gene III; Qβ3 capsid protein (as long as dimerization is not interfered with); and P22 tailspike protein (Carbonell and Villaverde, Gene 176:225, 1996). Techniques for inserting a foreign coding sequence into a phage gene sequence are well known to one of ordinary skill in the art (see e.g., Sambrook et al., Molecular Cloning: A Laboratory Approach, Cold Spring Harbor Press, NY, 1989; Ausubel et al., Current Protocols in Molecular Biology, Greene Publishing Co., NY, 1995).

In the preferred filamentous phage system, a wide range of vectors is available (see, Kay et al., Phage Display of Peptides and Proteins: A Laboratory Manual, Academic Press, San Diego, 1996). The most common vectors contain inserts in the gene III or gene VIII. Furthermore, a foreign gene can be inserted directly into a phage genome or into a phagemid vector. Methods of propagation of filamentous phage and phagemids are well known to one of ordinary skill in the art.

Filamentous phage vectors generally fall into two categories: phage genomes and phagemids. Either type of the vectors may be used within the context of the present invention. Many such commercial vectors are available. For example, the pEGFP vector series (Clontech; Palo Alto, Calif.), M13mp vectors (Pharmacia Biotech, Sweden), pCANTAB 5E (Pharmacia Biotech), pBluescript series (Stratagene Cloning Systems, La Jolla, Calif.), pComb3 and M13KE (New England Biolabs), and the like may be used. One particularly useful commercial phagemid vector is PEGFP-N1, which contains a green fluorescent protein (GFP) gene under control of the CMV immediate-early promoter. This plasmid also includes an SV40 origin of replication to enhance gene expression by allowing replication of the phagemid to high copy numbers in cells that make SV40 T antigen.

Other vectors are available in the scientific community (see, e.g., Smith, Vectors: A Survey of Molecular Cloning Vectors and their Uses, Rodriquez and Denhardt (eds.), Butterworth, Boston, pp 61-84, 1988) or may be constructed using standard methods (Sambrook et al., Molecular Biology: A Laboratory Approach, Cold Spring Harbor, N.Y., 1989; Ausubel et al., Current Protocols in Molecular Biology, Greene Publishing, NY, 1995), guided by the principles discussed below.

The source of the ligand (e.g., gene, gene fragment, peptide encoding nucleic sequence, chemically conjugated peptide or protein, or, non-covalently conjugated peptide or protein) may be, for example, derived from a cDNA library, antibody library, or random peptide library. Alternatively, the ligand may be from a library of random or selective mutations of a known ligand. In an additional alternative, the ligand may be from a library of a known receptor or cell surface binding agent. For example, the library may contain a subset of peptides that are known to bind to the FGF or EGF receptor, but have unknown gene delivery and expression characteristics (i.e., transduction capacity). Further, the ligand may be from a library of single chain antibodies, Fab fragments or other antibody fragments. Virtually any peptide or polypeptide that can be attached to the surface via covalent or non-covalent attachment or via genetic fusion of the nucleic acid sequence encoding the peptide or polypeptide of interest can be a putative ligand. Other ligands may include randomly or selectively cleaved protein fragments.

When a cDNA library is used, the starting cDNAs are synthesized from mRNAs isolated from a tissue or cell line from which a desired ligand originates. The cDNAs are then amplified using primers containing sequences of appropriate restriction enzyme sites for insertion into a desired vector. Alternatively, cDNAs from commercially available cDNA libraries (e.g., Clontech; Palo Alto, Calif.) may be amplified for insertion into a vector.

Similarly, libraries of antibody fragments can be made from mRNAs isolated from the spleen cells of immunized animals (immunized, for example, with whole target cells or membranes) or through subcloning of existing antibody libraries made from immunized or naive animals. Random peptide encoding sequences are subcloned from libraries that are commercially available (New England Biolabs; Mass.) or can be synthesized and cloned using previously described methods (see, Kay et al., supra).

Phage display libraries of random or selective mutations of known ligands (referred to herein as a “mutein library”) for improved gene delivery are performed in the same manner as described for screening random peptide libraries. Random mutations of a native ligand gene may be generated using DNA shuffling as described by Stemmer (Nature 370:389-391, 1994). Briefly, in this method, the native ligand gene is amplified and randomly digested with DNase I. The resultant 50-300 base pair fragments are reassembled by amplification that is performed with no primers and using a Taq DNA polymerase or the like. The high error rate of the Taq DNA polymerase or the like introduces random mutations into the fragments that are reassembled at random thus introducing combinatorial variations of different mutations distributed over the length of the native ligand gene. Error prone amplification may alternatively be used to introduce random mutations (Bartell and Szostak, Science 261:1411, 1993). The ligand may be mutated by cassette mutagenesis (Hutchison et a., in Methods in Enzymology 202:356-390, 1991), in which random mutations are introduced using synthetic oligonucleotides and cloned into the ligand to create a library of ligands with altered binding specificities. Additional mutation methods can be used. Some additional methods are described in Kay et al., supra. Further, selective mutations at predetermined sites may be performed using standard molecular biological techniques (Sambrook et al., Molecular Cloning: A Laboratory Manual, Cold Spring Harbor Press, 1989).

If a cDNA library cannot be generated because, for example, the source of the desired ligand is not available or unknown, random peptide libraries or a cDNA library from placenta may be used as a starting point for screening. Methods for construction of random peptide libraries may be found, for example, in Kay et al., supra. Briefly, random peptides that are encoded by DNAs assembled from degenerate oligonucleotides are inserted into one of the bacteriophage vectors described herein. Several different strategies may be used to generate random peptides or peptide encoding sequences. For example, triplets of NNN, wherein each N is an equimolar representation of all four nucleotides, will generate all 20 amino acids as well as 3 stop codons. Alternative strategies use NN(G/T) and NN(G/C), which results in 32 codons that encode all 20 amino acids and only 1 stop codon. Other strategies utilize synthesis of mixtures of trinucleotide codons representing all 20 amino acids and no stop codons. Once the oligonucleotides are synthesized, they are assembled as double strands by a variety of schemes, one of which involves synthesis of the complementary strand (see, Kay et al., supra).

When a nucleic acid molecule encoding a ligand, as described above, is fused with the coat protein of the ligand display genetic packages, including phagemids or phage genomes, the expression of the ligand frequently requires that the nucleic acid molecule encoding the ligand be fused in-frame with the coat protein. For example, when it is important to display a specific or known ligand on a genetic package, the ligand should be fused in-frame with the coat protein such that the coat protein-ligand fusion comprises the desired ligand sequence. In addition, even when random or unknown cDNAs or nucleic acid sequences are fused to a coat protein, it may be desirous to fuse the cDNAs in-frame to the coat protein. Examples of situations where in-frame fusions are preferred include when a library of cDNAs is being screened to identify a naturally occurring ligand and when out-of-frame fusions result in the expression of a coat protein-ligand fusion protein that is truncated due top the presence of stop codons in the out-of-frame ligand-encoding nucleic acid.

One way to increase the percentage of in-frame inserts in the ligand display genetic packages is to enrich for in-frame inserts prior to or concurrent with fusion with the coat proteins. The present invention provides a method for enriching in-frame inserts by utilizing a plasmid vector in which a coat protein signal sequence is fused to a selectable marker. When all DNA inserts are inserted into the plasmid vector between the coat protein signal sequence and the selectable marker, only the vectors containing an in-frame insert express the selectable marker. Typically, each vector contains one insert.

In certain embodiments, all DNA inserts can be inserted into the in-frame vector first, followed by selection of the in-frame inserts by transforming a host bacterium and culturing the bacteria in the presence of the appropriate selection (e.g., gene expression, fluorescence, antibiotic resistance, etc.). One of ordinary skill in the art would readily appreciate that the signal sequence can be derived from many proteins, so long as the signal sequence is suitable for a desired host bacterium. For instance, the coat proteins that may be used in the plasmid vector for enriching in-frame inserts include, without limitation, M13 gene III, gene VI, gene VII, gene VIII, and gene IX; fd minor coat protein pIII (Saggio et al., Gene 152:35, 1995); lambda D protein (Sternberg and Hoess, Proc. Natl. Acad. Sci. USA 92:1609, 1995; Mikawa et al., J. Mol. Biol. 262:21, 1996); lambda phage tail protein pV (Maruyama et al., Proc. Natl. Acad. Sci. USA 91:8273, 1994; U.S. Pat. No. 5,627,024); fr coat protein (WO 96/11947; DD 292928; DD 286817; DD 300652); φ29 tail protein gp9 (Lee, Virol. 69:5018, 1995); MS2 coat protein; T4 small outer capsid protein (Ren et al., Protein Sci. 5:1833, 1996), T4 nonessential capsid scaffold protein IPIII (Hong and Black, Virology 194:481, 1993), or T4 lengthened fibritin protein gene (Efimov, Virus Genes 10:173, 1995); PRD-1 gene III; Qβ3 capsid protein (as long as dimerization is not interfered with); and P22 tailspike protein (Carbonell and Villaverde, Gene 176:225, 1996).

In certain embodiments, it may be beneficial to utilize antibiotic resistance as the selectable marker. Furthermore, when the selectable marker is fused to a signal sequence that directs the marker to a particular site or region of a cell, it is important that the marker is active in the site. For example, when a marker is fused to a signal sequence, such as pIII, which directs a fused marker to the periplasm, it is important to use a marker that is active in the periplasm, such as β-lactamase. In one preferred embodiment, the selectable marker is the ampicillin resistance gene (β-lactamase). In other embodiments, other markers active in the periplasm may also be used. One of skill in the art could determine an appropriate marker to use with a given signal sequence, based upon information known in the art.

In addition to a ligand/coat protein fusion, in one aspect, a phage vector may contain a gene whose product can be detected or selected for. As referred to herein, a “reporter or marker” gene is one whose product can be detected, such as by immunodetection, fluorescence, enzyme activity on a chromogenic or fluorescent substrate, and the like, or selected for by growth conditions. Such reporter genes include, without limitation, green fluorescent protein (GFP), β-galactosidase, chloramphenicol acetyltransferase (CAT), luciferase, neomycin phosphotransferase, secreted alkaline phosphatase (SEAP), and human growth hormone (HGH). Selectable markers include genetic resistances to drugs, such as ampicillin, neomycin (G418), hygromycin, and the like. However, the present invention is not limited to these markers, as one of ordinary skill in the art could readily envision using any detectable product that allows one to distinguish a cell in which the transgene was introduced and/or expressed. For example, the gene or transgene may be a structural gene that is heterologous or endogenous to the host. If the transgene is endogenous to the host, detection may be done by comparison with a control of untreated cells. Further, as noted above, RNA derived from a transduced gene may be detected by RT-PCR, even absent a phenotypic change in the cell. In addition, any DNA sequence of the vector that is not normally expressed in the cell comprising an internalized ligand displaying genetic package may be directly selected by methods of detecting specific DNA sequences, such as Hirt extraction, PCR, and rolling circle amplificiation (using, e.g., phi29 or exo(−)BST DNA polymerase, etc.), for example. Direct detection of exogenous DNA sequence circumvents the need for expression of a transgene in the host or target cell and allows detection absent a phenotypic or observable change in the cell.

In order to be expressed, a marker gene is in operative linkage with a promoter in a vector. Any promoter that is active in the cells to be transfected can be used. The vector should also comprise a viral origin of replication and a packaging signal for assembling the vector DNA with coat proteins.

Most applications of the present invention will involve transfection of mammalian cells, including human, canine, feline, equine, and the like. However, several embodiments of the present invention utilize expression in non-mammalian cells (e.g., fungal, yeast, bacteria, and plant). The choice of a promoter will depend in part upon the targeted cell type. Promoters that are suitable within the context of the present invention include, without limitation, constitutive, inducible, tissue specific, cell type specific, temporal specific, or event-specific, while constitutive promoters are preferred.

Examples of constitutive or nonspecific promoters include SV40 early promoter (U.S. Pat. No. 5,118,627), SV40 late promoter (U.S. Pat. No. 5,118,627), CMV early gene promoter (U.S. Pat. No. 5,168,062), bovine papilloma virus promoter, and adenovirus promoter. In addition to viral promoters, cellular promoters are also amenable within the context of this invention. In particular, cellular promoters for so-called housekeeping genes are useful (e.g., β-actin). Viral promoters are generally stronger than cellular promoters.

Additional promoters known and available to one of ordinary skill in the art may also be useful within the context of the present invention. For example, if display packages are to be used to transduce yeast, a yeast promoter will be required. Some yeast specific promoters are available in the context of a number of commercially available vectors from a variety of sources including Clontech, (Palo Alto, Calif.) and Invitrogen (Carlsbad, Calif.). Further, if transduction of bacteria is of interest, a number of bacterial vectors are available of which most are derived from the pUC lineage. In addition, a variety of plant vectors and promoters are available. For example, general descriptions of plant expression vectors and reporter genes can be found in Gruber et al. (Vectors for Plant Transformation, in Methods in Plant Molecular Biology & Biotechnology, Glich et al. (Eds.) pp. 89-119, CRC Press, 1993). Promoters useful within the context of the present invention include both constitutive and inducible natural promoters, as well as engineered promoters. Such promoters may be obtained from plants, viruses or other sources, and include, but are not limited to, those described herein, such as 35S promoter of cauliflower mosaic virus (CaMV). Typically, for plant expression vectors, suitable promoters include 35S RNA and 19S RNA promoters of CaMV (Brisson et al., Nature 310:511, 1984; Odell et al., Nature 313:810,1985), the full-length transcript promoter from Figwort Mosaic Virs FMV) (Gowda et al., J. Cell Biochem. 13D: 301, 1989 and U.S. Pat. No. 5,378,619), and the coat protein promoter to TMV (Takamatsu et al., EMBO J 6:307,1987). Alternatively, plant promoters such as the light-inducible promoter from the small subunit of ribulose bis-phosphate carboxylase (ssRUBISCO) (Coruzzi et al., EMBO J3:1671, 1984; Broglie et al., Science 224:838,1984); mannopine synthase promoter (Velten et al., EMBO J 3:2723, 1984); nopaline synthase (NOS) and octopine synthase (OCS) promoters (carried on tumor-inducing plasmids of Agrobacterium tumefaciens) or heat shock promoters (e.g., soybean hsp17.5-E or hsp17.3-B) (Gurley et al., Mol. Cell. Biol. 6:559, 1986; Severin et al., Plant Mol. Biol. 15:827, 1990) may be used. See PCT Publication WO 91/19806 for a review of a variety of known plant promoters which are suitable for use within the context of the present invention.

In preferred embodiments, the phage has an origin of replication suitable for the transfected cells. Viral replication systems, such as EBV ori and EBNA gene, SV40 ori and T antigen, or BPV ori, may be used. Mammalian replication systems may also be used. Expression of therapeutic or reporter genes from a phage genome may be enhanced by increasing the copy number of the phage genome. For example, the SV40 origin of replication is used in the presence of the SV40 T antigen to cause several hundred thousand copies. A T antigen gene may be already present in cells, introduced separately into cells, or included in a phage genome under the transcriptional control of a suitable cell promoter. Other viral replication systems for increasing copy number can also be used, such as EBV origin and EBNA.

As noted above, phagemid vectors may also be utilized in the practice of the present invention. Phagemid vectors are plasmid vectors that contain filamentous phage sequences and, therefore, can be packaged into phage particles when complementing phage structural proteins are provided in trans by a helper phage. In conventional phagemid systems, both phagemid and helper phage encode pIII coat protein. Both wild type and pIII-fusion protein are displayed on the resulting phagemid particles with the phagemid encoding the pIII fusion protein and the helper encoding wild type pIII protein. This results in a monovalent display of peptides or proteins with often less than one recombinant pIII fusion protein displayed per phage particle. Many antibody libraries are made in phagemid vectors because monovalent display is advantageous for selecting high affinity antibodies over lower affinity antibodies which in a high valence system might be selected on the basis of avidity. While similar concerns may justify the use of monovalent display vectors in the practice of the present invention, current data suggests, however, that multivalent display is important for mammalian cell internalization. (Larocca et al., FASEB J. 13:727-734, 1999; Larocca et al., Human Gene Therapy 9:2393-2399, 1998; Becerril et al., Biochem. Biophys. Res. Comm. 255:386-393; Ivanenkov et al., Biochim. Biophys. Acta 1448:450-462, 1999). Thus, a phagemid vector with multivalent display would likely be more useful for selecting certain internalizing phage in mammalian cells.

Multivalency in a phagemid system may be provided by a variety of techniques including by rescuing with a helper having a gIII deletion (or gVI, gVII, gVIII, gIX deletion or similar phage coat protein, depending on the phagemid). Accordingly, rescuing with a gIII deleted helper can alter the valency of phagemid vectors. Rakonjac and colleagues have developed host strains which allow production of the gIII deleted helper phage with very low background of gIII containing recombinants (as low as 1 in 10⁹). See, e.g., Rakonjac et al., Gene 198:99-103, 1997. In this system, there is little or no wild type pIII provided by the helper phage so that each phage displays multiple copies of the pIII fusion derived from the phagemid. Thus, in one embodiment, the present invention utilizes a vector containing an EGF-pIII fusion protein from a phage vector, MG4-EGF (the vector MG4 is constructed by reversing the orientation of the SV40ori/CMV/GFP expression cassette in the MEGFP3 (MG3) phage vector. The MG4-EGF has the EGF encoding gene inserted “in-frame” at the pst1/Nco1 sites in MG4) and, similarly, the control vector containing the CMV/SV40ori/GFP cassette and the pIII gene from the control phage. The pIII genes are under the transcriptional control of the inducible lac promoter to minimize synthesis of pIII protein in the absence of a suitable inducer (e.g., IPTG).

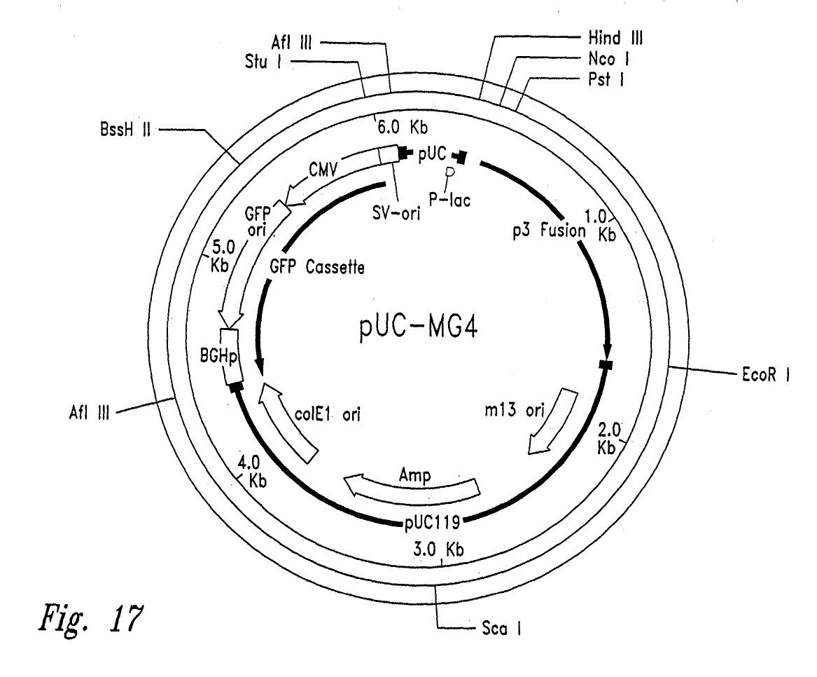

Construction and utilization of multivalent phagemid vectors are within the knowledge of those of skill in the art. Briefly, the backbone of the above phagemids can be easily constructed from pUC119 (purchased from ATTC), which contains an M13 phage origin of replication and an ampicillin resistance gene. The CMV/SV40ori/GFP gene is PCR amplified using primers that incorporate an AflIII endonuclease site and is inserted into the unique AflIII site in pUC119 to create pUC-GFP. The pIII and EGF-pIII fusion genes are subcloned from MG4 and MG4-EGF by PCR amplification using primers that encode HindIII and EcoR1 endonuclease sites and inserted into pUC-GFP at the unique HindIII and EcoR1 sites in the multicloning site to create pUC-MG4 (FIG. 17) and pUC-MG4-EGF. The phagemid DNA constructs are used to transform host bacteria (XL-1 Blue; Stratagene, San Diego, Calif.) that contain a lac IQ repressor protein gene to suppress expression of the pIII or EGF-pIII fusion gene in the absence of IPTG. Phagemid particles are rescued from transformed bacteria with a wild type helper phage (i.e. R408 or VCSM13) or a gIII deleted helper (i.e. R408d3 or VCSM13d3, Stratagene, San Diego, Calif.) and tested on COS1 cells for phage mediated transduction efficiency as measured by % GFP positive cells. The results of these experiments (not shown) indicate that the multivalent phage (VCSM13d3 rescued) are about 2 orders of magnitude more efficient at transducing COS1 cells than the monovalent display phagemid particles (wild type rescued). Western blot analysis (not shown) of CsCl phagemid particles reveals that about 50% of the pIII protein displayed on the VCSM13d3 rescued phagemid particles is fused to EGF; the remaining pIII protein is likely derived from a proteolytic cleavage of the fusion protein. Analysis of the monovalent phagemid particles shows that more than 66% of the displayed pIII is the length of wild type pIII and of MG4-EGF phage shows that 60-80% of the pIII displayed is full length. Accordingly, such data demonstrate that multivalent display of the targeting ligand facilitates uptake of the ligand targeted phage via cognate receptor mediated endocytosis and that it is feasible to construct and use a multivalent phagemid system for phage mediated mammalian cell transduction.

In yet further embodiments, ligand fusions to a truncated pIII, pVI, pVII, pVIII, pIX, or other appropriate phage coat gene may be utilized. Several advantages become apparent when expressing displayed ligands as fusions to a truncated phage coat gene. For example, rescue by a helper phage is simplified, because there is no interference of phagemid derived pIII with the infection by the helper phage. Thus, multivalent phagemid yields will likely be increased by infection with a helper phage after induction of the phagemid pIII fusion gene (not possible with fusions to full length pIII because of interference). Additionally, some ligands may be more efficiently expressed as a fusion to truncated pIII.

Phagemid vectors having truncated fusions to pIII or pVIII are easily produced by those of ordinary skill in the art. Briefly, a phagemid vector is created with a pIII gene having only domain 2 of the pIII (preferably starting from amino acids 198-250 and ending at amino acid 406), as described by Doftavio (in Phage Display of Peptides and Proteins, Kay et al. (Eds.) San Diego, Academic Press, 1998). When this phagemid is rescued with the R408d3 or VCSMI 3d3 helper phage, the resulting phagemid particles are no longer infective in bacteria, since domain 1 of pIII is required for infectivity. However, these particles can be used for non-bacterial cell transduction and subsequent rescue of ligand encoding sequences, as described herein. Further, to enhance ligand recognition, it may be beneficial to fuse the sequence encoding the peptide or protein ligand or the peptide or protein ligand itself to domain 2 of pIII via a linker molecule (e.g., gly₄ser, heterobifunctional linkers, other chemical linkers, and the like).

In a further embodiment, a viral replication system, such as an SV40 based shuttle vector, can be used as a phagemid. Accordingly, cells infected by the ligand-expressing phage package the phagemid DNA into SV40 viral particles. These viral particles infect neighboring cells, thus spreading the phagemid DNA. Following growth in culture, a dish of cells contains millions of copies of the original phagemid, thereby enriching the population of internalized ligand encoding genetic packages several fold. The cell lines used with such a system can either be transfected with DNA sequences encoding SV40 small and large T-antigens or can contain the proteins through delivery with fusion constructs, such as VP22. VP22 is a herpes virus structural protein that is exported from cells and spreads to neighboring cells where it concentrates in the nucleus (Elliot and O'Hare, Cell 88:223-233, 1997). VP22-SV40 T antigen fusion protein-encoding vectors exist in the art and are available from Invitrogen Corp., such as the pVP22myc-His vector. A vector useful in this application ideally contains an f1, M13 or comparable phage origin, a coat protein fusion cassette (pIII or pVIII), an SV40 late region genes, and an SV40 origin.

Similarly, an adeno-associated virus/phage (AAV-phage) hybrid vector may be used to achieve the same amplified ligand production. An AAV-phage hybrid vector combines selected elements of both vector systems, providing the vector that is simple to produce in bacteria without constraint by capsid packaging limit, while allowing infection of quiescent cells and integration into the host chromosomes. Vectors containing many of the appropriate elements are readily available, and can be further modified by standard methodologies to include necessary sequences. For example, the phagemid pMV/Svneo, ATCC Accession No. 68065, contains AAV ITR sequences and an F1 origin of replication. In addition the vector pAV.CMV.LacZ provides many of the appropriate elements. Fisher et al., J. Virol. 70:520-532, 1996.

Adeno-associated virus (AAV) is a defective member of the parvovirus family. The AAV genome is encapsulated as a single-stranded DNA molecule of plus or minus polarity (Berns and Rose, J. Virol. 5:693-699, 1970; Blacklow et al., J. Exp. Med. 115:755-763,1967). Strands of both polarities are packaged, but in separate virus particles (Berns and Adler, Virology 9:394-396, 1972) and both strands are infectious (Samulski et al., J. Virol. 61:3096-3101, 1987). The single-stranded DNA genome of the human adeno-associated virus type 2 (AAV2) is 4681 base pairs in length and is flanked by inverted terminal repeated (ITR) sequences of 145 base pairs each (Lusby et al., J. Virol. 41:518-526,1982; Muzyczka, Curr. Top. Microbiol. Immunol. 158:97-129, 1992). In addition, the viral rep protein appears to mediate non-homologous recombination through the ITRs (Giraud et al., J. Virol. 69:6917-6924, 1995; Linden et al., Proc. Natl. Acad. Sci. USA 93:7966-7972, 1996). Accordingly, as parvoviral genomes have ITR sequences at each end which play a role in recombination and which are generally required for parvoviral replication and packaging, the vectors of the present invention generally contain all or a portion of at least one of the ITRs or a functional equivalent thereof.

Adeno-associated viruses may be readily obtained and their use as vectors for gene delivery has been described in, for example, Muzyczka, Curr. Top. Microbiol. Immunol. 158:97-129, 1992; U.S. Pat. No. 4,797,368, and PCT Application WO 91/18088. Construction of AAV vectors is described in a number of publications, including U.S. Pat. No. 5,173,414; Lebkowski et al., Mol. Cell. Biol. 8:3988-3996, 1988; Tratschin et al., Mol. Cell. Biol. 5(11):3251-3260; Hermonat and Muzyczka, Proc. Nat'l. Acad. Sci. USA 81:6466-6470,1984; U.S. Pat. Nos. 5,871,982, 5,773,289, 5,843,742, and 5,474,935; and PCT Application Nos. WO 98/45462 and WO 98/48005, all of which are incorporated herein by reference.

AAV-2 can be propagated as a lytic virus or maintained as a provirus integrated into host cell DNA (Cukor et al., in “The Parvoviruses,” Berns ed., Plenum Publishing Corp., N.Y. pp. 33-66, 1984). Although under certain conditions AAV can replicate in the absence of a helper virus (e.g., Yakobson et al., J. Virol. 61:972-981, 1987), efficient replication requires coinfection with either an adenovirus (Atchinson et al., Science 194:754-756, 1965; Hoggan, Fed. Proc. Am. Soc. Exp. Biol. 24:248,1965; Parks et al., J. Virol. 1:171-180,1967), herpes simplex virus (Buller et al., J. Virol. 40:241-247,1981), cytomegalovirus, Epstein-Barr virus, or a vaccinia virus. Hence, AAV is classified as a “defective” virus.

An AAV-phage hybrid vector for ligand identification generally comprises an F1, M13 or comparable origin, a coat protein fusion cassette (e.g., pIII-ligand, pVI-ligand, pVII-ligand, pVIII-ligand, pIX-ligand, or other phage coat-ligand combinations), a bacterial gene which facilitates selection, such as ampicillin resistance, and the two AAV ITRs (or functional equivalents thereof), between which is inserted a promoter-driven reporter or selectable gene. The hybrid phage can then be used to transduce cells. Positive cells are identified and ligand sequences are amplified by PCR using the ITRs or coat protein gene as the template sequence. This amplified ligand fusion construct can then be sub-cloned into other vectors for further rounds of transfection, selection, and ligand sequence identification.

In related embodiments, the present invention provides an AAV-phage vector that is designed to produce functional AAV viral particles upon co-infection with a “rescue” virus, such as an adenovirus. In this embodiment, the AAV-phage particle enters a cell as a “phage particle”, but once inside, produces AAV particles that infect surrounding cells. This method can thus be used to amplify gene delivery effectiveness in a target organ or tissue. Briefly, a ligand displaying phage particle containing an AAV-phage vector which contains a transgene of interest as well as ligand fused to the coat protein is used to transduce a cell. Concurrent with or subsequent to contacting the cells with the transgene containing AAV-phage particle, a ligand displaying helper AAV-phage is also used to supply the rep and cap genes for viral particle formation. Following transduction with the appropriate bacteriophage, the cell is infected with a “rescue” virus, such as an adenovirus, which allows viral particles to form and infect neighboring cells. Alternatively, the rep and cap functions can be supplied on the helper adenoviral genome.

In another aspect, mutant coat proteins or additional components or methods may be used to increase transduction efficiency. A particularly preferred manipulation is to mutagenize a coat protein so as to facilitate uncoating upon cellular internalization. For example, the filamentous phage coat protein VIII encoding gene can be mutagenized such that it is encodes a slightly unstable protein and thus allows more rapid uncoating and increased transduction capacity. Accordingly, one of ordinary skill in the art would readily recognize that given the teachings presented herein, mutations may be selected for using reporter gene expression.

In various embodiments, when utilizing filamentous phage or another single-stranded DNA vector, transduction may be enhanced by facilitating the conversion of the single-stranded DNA to a double-stranded DNA. The mechanism for conversion of a single-stranded phage DNA to a double-stranded DNA is analogous to that which occurs during infection with single-stranded DNA genome of a parvovirus such as an adenoassociated virus (AAV). In the case of a recombinant AAV, conversion to dsDNA is a rate-limiting step for efficient transduction. The E4 or f6 gene product provided by helper Adenovirus or on a separate expression plasmid provides this function and increases transduction efficiency between 100-1000 fold. Accordingly, a variety of genotoxic treatments including gamma radiation, UV, heat shock, and DNA synthesis inhibitors and topoisomerase inhibitors (e.g., hydroxyurea, camptothecin, etoposide, and the like (see, e.g., Ferrari et al., J. Virol. 70:3227-3234, 1996; Alexander et al., J. Virol. 68:8282-8287, 1994; Russell et al., PNAS 92:5719-5723, 1995)) will increase rAAV transduction efficiency in the absence of infection by a helper virus. Similar treatments can also increase the transduction efficiency of recombinant phage vectors and thus may be utilized in the practice of the present invention. Accordingly, any such treatment or agent that facilitates DNA repair and/or facilitates the conversion of a single stranded DNA to a double stranded DNA is considered a genotoxic agent or treatment as that term is utilized herein.

In other embodiments, peptides or other moieties that allow or promote the escape of the vectors (and any molecule attached thereto or enclosed therein) from the endosome and/or target the vectors to the nucleus are incorporated and expressed on or attached to the surface of the ligand displaying genetic package (e.g., bacteriophage). Such “other moieties” include molecules that are not themselves peptides but which have the ability to disrupt the endosomal membrane, thereby facilitating the escape of the vector, and molecules that otherwise mimic the endosomal escape properties of the within-described peptide sequences (see, e.g., published PCT Application No. WO 96/10038, the disclosure of which is incorporated by reference herein).

Peptide sequences that confer the ability to escape the endosome are particularly preferred. Such sequences are well known and can be readily fused or conjugated covalently or genetically to a coat protein, such as genes III, VI, VII, VII, and IX or similar coat protein encoding genes of filamentous phage. Although fusion of one or more peptide sequences to a coat protein is described herein as a preferred embodiment, it should be understood that other methods of attachment—and other moieties besides peptides—are useful as well. Further, as those of skill in the art can readily appreciate, the present invention may be utilized to screen for peptide sequences that enhance transduction via endosomal escape or nuclear targeting when combined with ligand targeting agents. Accordingly, dual display peptides may be utilized for such screening, wherein one coat fusion or conjugate represents a known ligand and the other coat protein fusion or conjugate represents the sequence to be screened.

As with single display vectors, novel peptides or variants may be selected through rounds of cell contact and recovery of cells expressing the reporter gene, followed by identification of the encoded ligand.

In yet other embodiments, combinations of elements that facilitate transduction may be utilized. For example any combination of genotoxic treatment, endosomal escape peptides, and nuclear targeting may be used. In one example, UV and heat shock or heat shock and an endosomal escape peptide or nuclear targeting peptide may be utilized.

In yet another embodiment, cell attachment moieties (e.g., peptides) may be chemically conjugated, non-covalently, (e.g., electrostatic, antibody-antigen, biotin-streptavidin, etc.) or genetically fused to the exterior of the ligand display package. For example, many animal viruses encode sequences for both general cell binding and sequences specific for internalization. In this regard, adenovirus particles use both the knob protein (for receptor binding) and integrin binding for internalization. Highly charged proteins such as polylysine also facilitate binding to the negatively charged cell membrane, but may also result in undesirable non-specific transduction of any cell. Thus, when non-specific transduction is sought, highly charged peptides such as polylysine, histone, etc. may be used. However, in the practice of the present invention preferred peptides are those that will bind to cells at low affinity and facilitate internalization only in the presence of a second displayed ligand (e.g., EGF). Examples include L-selectin, the sequences encoding known heparin binding peptides (e.g., residues 65-81 from FGF2 which have been implicated in heparin binding (Imamura et al., Biochem. Biophys. Acta 1266:124-130, 1995)) and the heparin binding sequences identified in angio-associated migratory cell protein (Beckner et al., Cancer Res. 55:2140-2149, 1995) that fit a heparin binding consensus RRXRRX (SEQ ID NO: 18) (Cardin and Weintraub, Arteriosclerosis 9:21-32, 1995) and RGD containing sequences (Pierschbacher and Ruoslahti, Nature 309:30-33,1984) that bind cell surface integrins.

As one of ordinary skill in the art can readily appreciate, the selection methodologies described herein may be utilized to screen for additional cell attachment peptides. Briefly, cell attachment peptides may be conjugated or genetically fused to the exterior of a ligand display package. Typically, the cell attachment moiety will be attached to a display package such as a dual display phage such that an internalizing ligand and the cell attachment moiety will be jointly displayed. Accordingly, such joint display allows one of skill in the art to detect a change in transduction efficiency between packages displaying internalizing ligands alone and co-display in the presence of a cell attachment moiety (e.g., peptide).

In one example, to allow independent binding of the EGF-pIII fusion protein and the accessory cell attachment peptide, the cell attachment peptides are fused or conjugated to the phage major coat protein, pVIII (a small protein that makes up the tubular protein sheath that encapsulates the phage genome of which there are about 2700 copies per particle). Further, it has been established that pVIII can tolerate the addition of small peptides on its N-terminus (Ilyichev et al., FEBS Lett 301:322-324, 1992; Makowski Gene 128:5-11, 1993) which is used to produce the so-called “landscape phage” (Petrenko et al., Protein Eng. 9:797-801, 1999). Small peptides (˜6-8aa) may be fused directly to the N-terminus or made as substitutions in the middle of pVIII as described by Petrenko (supra). For larger peptides or where more than one peptide is to be inserted into or conjugated to the coat, a wild type pVIII protein may be included in the phage system as described for the dual display phagemid. Once sequences are found that enhance transduction efficiency in a representative cell line such as COS1 or other cells, the sequences may be further optimized by mutation and further selection by the ligand identification methods described herein.

As noted above, dual display vectors are useful within the context of the present invention. In dual display embodiments, additional elements displayed on a coat protein (e.g., pIII, pVI, pVII, pVIII and/or pIX and the like), such as an endosomal escape sequence, nuclear trafficking sequences or random peptide sequence, may be incorporated into or conjugated to the phage particle to enhance phage mediated transduction or to test for enhanced transduction. These elements enhance transduction by facilitating cell binding, endosomal escape, nuclear localization, and other points along the transduction pathway that are currently rate limiting. Accordingly, it would be advantageous to display separate elements on pIII and pVIII (or analogous exterior proteins, e.g., pVI or others) such that, for example, the targeting ligand is expressed on pIII and the endosomal escape sequence on pVIII. Such separation may also minimize the possibility of interference of the function of one element with the other. Thus, a phagemid vector can be constructed that allows fusion and display of distinct peptides or proteins on both pIII and pVII. For example, in one embodiment, only one pIII gene (the fusion) may be present when phage rescue is performed with pIII deleted helper phage. However, two pVIII genes are present: one on the phagemid (the pVIII fusion) and one (wild-type) on the helper phage. Thus, the vector is designed to display a peptide or protein ligand on pIII and additional accessory peptide(s) or protein(s) on pVIII as a mosaic with wild type pVIII. Further, as there may exist an upper limit to displaying peptides on pVIII because peptides larger than about 8 residues interfere with assembly of the phage particle (Ilyichev et al. (supra); Makowski, (supra)), displaying the accessory protein as a mosaic allows for proper particle assembly and the display of peptides larger than 6-8 residues on the major coat protein, pVIII.

Thus, an example of a dual display filamentous phage presents a ligand (e.g., FGF) as a fusion to gene III and an endosomal escape peptide fused to gene VIII. The locations of the ligand and escape sequences are interchangeable. Escape sequences that are suitable include, without limitation, the following exemplary sequences: a peptide of Pseudomonas exotoxin (Donnelly, J. J., et al., PNAS 90:3530-3534, 1993); influenza peptides such as the HA peptide and peptides derived therefrom, such as peptide FPI3; Sendai Virus fusogenic peptide; the fusogenic sequence from HIV gp1 protein; Paradaxin fusogenic peptide; and Melittin fusogenic peptide (see WO 96/41606).

Another sequence that may be included in a vector is a sequence that facilitates trafficking proteins into the nucleus. These so-called nuclear translocation or nuclear localization sequences (NLS) are generally rich in positively charged amino acids. Because the carboxyl terminus of gene VIII protein of filamentous phage already carries a positive charge, increased charge and likeliness of nuclear transport may be enhanced by fusing known mammalian cell NLS sequences to the gene VII protein. NLS fusions to other coat proteins of filamentous phage may be substituted.

Examples of NLS sequences include those resembling the short basic NLS of the SV40 T antigen; the bipartite NLS of nucleoplasmin; the ribonucleoprotein sequence A1; the small nuclear ribonucleoprotein sequence U1A, and human T-lymphocyte virus-1 Tax protein. Other useful NLS sequences include the HIV matrix protein NLS, the nuclear translocation components importain/hSRP1 and Ran/TC4, the consensus sequence KXX(K/R) (SEQ ID NO: 4) flanked by Pro or Ala, the nuclear translocation sequence of nucleoplasmin, or the NLS from antennapedia (see WO 96/41606).

Further, sequences which direct the genetic package to various cellular compartments may be useful within the context of the present invention. For example, while FGF appears to be trafficked to the nucleus via a nuclear localization-like peptide, EGF appears to be trafficked through the lysosome. Accordingly, in addition to a putative ligand, a lysosomal directing sequence may be incorporated into one of the coat proteins of the genetic package. Exemplary sequences in this regard are KCPL (SEQ ID NO: 11) which acts as a lysosomal targeting sequence (Blagoveshchenskaya et al., J. Biol. Chem. 273(43):27896-27903, 1998), the ubiquitin-dependent endocytosis motif DSWVEFIELD (SEQ ID NO: 12) (Govers et al., EMBO J. 18(1):28-36, 1999), and DQRDLI (SEQ ID NO: 13) or EQLPML (SEQ ID NO: 14) from MCHII which also target the lysosome (Kang et al., J. Biol. Chem. 273(32):20644-20652, 1998).

As described herein, the library is then propagated in the display phage by transfection of a suitable bacteria host (e.g., DH5αF′ for filamentous phage), and growing the culture, with the addition of a replication-competent helper virus (for phagemid vectors) if necessary, overnight at 37° C. The phage particles are isolated from the culture medium using standard protocols.

In certain embodiments, infection of mammalian cells with phage is performed under conditions that block entry of wild type phage into cells (Barry et al., Nature Med. 2:299-305, 1996). Phage are added directly to cells, typically at titers of ≦10¹² CFU/ml in a buffer, such as PBS with 0.1% BSA or other suitable blocking agents, and allowed to incubate with the cells at 37° C. or on ice. The amount of phage added to cells will depend in part upon the complexity of the library. For example, a phage display library containing 10⁵ members has each member represented 10⁶ times in 1 ml of a typical phage titer of 10¹¹ colony forming units/ml.

In order to enhance transduction with a ligand-displaying genetic package, transfection or infection enhancing reagents may be added to cells or packages during transfection or infection. Transfection agents may increase the rate of transduction or increase expression of a detectable marker by a transduced cell, for example. A variety of transfection enhancing reagents are known and available in the art, e.g. DEAE-dextran, and cationic lipid and non-lipid formulations. Preferably, the transfection enhancing reagents enhance ligand-specific transduction, rather than merely non-specific uptake by a cell rotissue. Such agents may be added either before, during, or following contacting a ligand displaying genetic package with a cell or tissue.

In one embodiment, phage-mediated cell transduction is enhanced with DEAE-dextran. Treatment of cells with phage in the presence of DEAE-dextran increased phage transduction and marker gene expression substantially, as described in Example 39. While not wishing to by bound by a particular theory, the DEAE-dextran may work by complexing with the phage particles and neutralizing the negatively charged particles, which would allow greater contact with the negatively charged cell surface. This theory is consistent with the observation that simply preincubating the cells with DEAE-dextran also increased transduction by subsequent addition of targeted phage (data not shown). In addition, DEAE-dextran may be facilitating endosomal escape and nuclear trafficking, which could also account for the increased gene expression per cell. Regardless of the enhancement mechanism, the addition of DEAE-dextran is particularly useful for assessing phage gene delivery in situations where transduction or gene expression is comparatively low in the absence of DEAE-dextran or another transfection-enhancing agent. For example, DEAE-dextran may be useful for assessing phage gene delivery in an in vivo gene activated matrix (GAM) model. GAMs include biocompatible matrixes that allows for cell ingrowth, for example, and are described in U.S. patent applications Ser. No. 08/631,334, Ser. No. 09/344,581, and Ser. No. 09/178,286, which are incorporated by reference in their entirety. Moreover, DEAE-dextran may also be useful for increasing plasmid expression in a GAM.

Accordingly, the methods of the invention may also comprise the addition of a transfection or infection-enhancing agent. In one embodiment, the transfection-enhancing agent is DEAE-dextran. DEAE-dextran may be added at any sutiable concentration, depending on the particular cell type, for example. Determining the appropriate concentration to use for a particular cell type or delivery vehicle is routine in the art. Examples of appropriate concentrations of DEAE-dextran include, but are not limited to, 0.3 ug/ml, 0.6 ug/ml, 1.0 ug/ml, 10 ug/ml, 25 ug/ml, 50 ug/ml, 100 ug/ml, 150, ug/ml, 200 ug/ml, and any integer value within. In one particular embodiment, the concentration is 0.6 ug/ml. In other embodiments, the concentration may be greater than 200 ug/ml.

II. Detection/selection of Transgene Expression

A ligand displaying genetic package or a library of the same is ultimately screened against a target tissue or cell line. Screening can be performed in vitro or in vivo. While combinatorial screening methods have been performed in the past, these methods are unable to determine the transduction capability of a displayed ligand (see, U.S. Pat. No. 5,733,731, incorporated herein by reference). The criteria for a positive “hit” in the present invention is that a phage must be able to bind, be internalized, and enable detection of the internalized ligand by detecting a selectable marker, such as, for example, by expressing the phage genomic DNA containing the reporter/selectable gene in the target or test cell or allowing direct nucleic acid detection (e.g., PCR, rolling circle amplification by phi 29 polymerase or similar polymerase, or DNA binding). In this regard, while not wishing to be bound to a particular theory, it is believed that the phage should bind, internalize, uncoat, translocate to the nucleus, and replicate, in order to express the gene or otherwise facilitate detection (however, translocation and uncoating may occur in any order). Accordingly, direct nucleic acid detection can detect both cytoplasmic and nuclear located nucleic acid molecules. Thus, in preferred embodiments, only sequences that reach the nucleus are selected.

In certain aspects of the present invention, a variety of modifications may be utilized to enhance detection. For example, nucleic acid sequences may be recovered from targeted cells by Hirt extraction, by PCR (full vector PCR or fragment PCR), by RT-PCR of the encoded RNA transcript of the encoded detectable marker, by utilizing a binding agent such as a primer or a nucleic acid binding domain that is labeled and allows separation once bound to phage nucleic acid sequences (e.g., the primer could be biotinylated and then mixed with a streptavidin conjugated magnetic bead, thereby allowing extraction of only the sequences, such as phage sequences, bound by the primer), by DNA amplification methods that use rolling circle methods such as phi 29 polymerase, available from Amersham (see, e.g., Dean et al., Genome Res, 11(6):1095-1099, 2001), and similar systems (e.g., exo(−)BST DNA polymerase). In addition, vectors for selection of in-frame inserts may be utilized to enhance selection of only in-frame sequences from cDNA, mutein, or other nucleic acid libraries.

In one embodiment, nucleic acid sequences trafficked to the nucleus may be directly detected by RT-PCR of RNA transcribed from the delivered nucleic acid sequence. Briefly, in such an embodiment, a vector sequence may be designed to encode a detectable marker linked genetically with a ligand and/or coat protein. In certain of these embodiments, the poly A tail of the detectable marker is removed such that a single RNA transcript may be utilized for RT-PCR and detection of the internalizing ligand. Those of ordinary skill in the art can readily perform RT-PCR based on known protocols and commercially available kits and supplies.

In certain embodiments wherein rolling circle polymerase amplification is utilized, such methods allow for improved means for selecting internalized phage from phage display libraries by direct amplification of phage DNA internalized in the cells of interest. Utilizing such rolling circle amplification and full-vector PCR overcomes the necessity of subcloning recovered DNA generated by fragment PCR into an additional vector for further rounds of phage display screening. In addition, rolling circle amplification eliminates artifacts associated with PCR and allows for amplification at a single temperature, thus increasing ease of use. Recovery of internalized phage is greatly improved by the amplification of circular phage DNA using phi 29 DNA polymerase, which replicates multiple branched copies of each genome, resulting in exponential amplification in a single temperature reaction. Zhang, W. et al., J. Clin. Microbiol. 40:128-32 (2002). Using this method, internalizing DNA is enriched over one million fold in one selection (data not shown). By comparison to affinity selection of a ligand by biopanning, a high affinity ligand (Kd=10 nM) is enriched about 40,000 after a single round, while a low affinity ligand (Kd=18 uM) is enriched only about 1000 fold when selected on a purified target. Kay, B. K. et al., Phage Display of Peptides and Proteins: A Laboratory Manual, ed. Kay, B. K. et al., San Diego: Academic Press, pp. 55-65 (1998). Such increased sensitivity in detecting internalizing phage permit the detection and recovery of internalizing phage after only one round of selection, rather than multiple selection rounds, as required for biopanning. Thus, the invention includes the selection and identification of ligands according to methods of the invention following only one round of selection, following two or fewer rounds of selection, following three or fewer rounds of selection, following four or fewer rounds of selection, following five or fewer rounds of selection, or following any number of rounds of selection fewer than the number required for the selection of any ligand by biopanning. In addition, in certain embodiments, the methods of the invention may provide enrichment of at least 1,000-fold, at least 5,000-fold, at least 10,000-fold, at least 20,000-fold, at least 50,000-fold, at least 100,000-fold, at least 250,000-fold, at least 500,000-fold, at least 1,000,000-fold, or enrichment by at least any integer value between 1,000 and 1,000,000-fold. In one embodiment, the invention provides greater than 1,000,000-fold.

Test cells may be any cells that express a receptor of choice or may be a cell type or source for which gene therapy is destined. Test cells may include any cell or tissue, particularly where the invention is being used to identify cells bound by a known ligand or to identify ligand/anti-ligand pairs. Thus, in some instances, the receptor may be unknown. In some cases, the selection method can be used to isolate a ligand for a receptor without a known ligand (orphan receptor) such as erbB3 or similar orphan receptor. Briefly, the orphan receptor is cloned into a mammalian expression vector that also contains a selectable drug resistance gene and transfected into mammalian cells, such as COS cells. Stable transfectants that overproduce the orphan receptor are selected by cultivation in the appropriate drug. This receptor-transformed COS cell line is then used as the cell line for selection of ligand-displaying phage.

In one aspect of the present invention, natural ligands or domains (i.e., a naturally occurring ligand for a cell surface protein that internalizes) are selected or identified. In this aspect, display of protein domains instead of full length cDNAs is utilized by fragmenting the cDNAs to an average size that would encode proteins ranging from about 50 to 900 amino acids (˜150 to 2700 base pairs). It is estimated that 80% of all active protein domains fall within this range. In addition to revealing active domains, fragmentation may allow more sequences to be displayed, because the smaller active peptide domains would be separated from sequences that inhibit display. Fragmentation is accomplished by using random primers and PCR amplification during cDNA synthesis or by DNAse 1 digestion of full-length cDNA. See, e.g., Cochrane et al., J. Mol. Biol. 297:89-97, 2000 and Santini et al., J. Mol. Biol. 282:125-135, 1998. Accordingly, the present invention has application in identifying cell-targeting ligands from cDNAs derived from various cell types.

In one embodiment, a library of cDNAs that are representative of the sequences encoding all the protein domains made by a cell type or tissue is utilized. Briefly, the library is constructed using random oligonucleotide primers that have extensions encoding restriction enzyme recognition sites, such that the final cDNA products can be digested with the restriction enzymes and ligated into an appropriate phage vector (e.g., MG4, pUC-MG4). For example, the first strand cDNA synthesis is primed with a random 6-mer oligonucleotide containing a Pst1 restriction site extension. The second strand synthesis is performed using a random 6-mer oligonucleotide extended with a Nco1 restriction endonuclease. In this manner the cDNA fragments are cloned into the Nco1/Pst1 sites in a vector in the 5′ to 3′ direction. Thus only three possible reading frames out of six (if read off both coding and complementary strands) are inserted into the vector and one or more of these orfs encodes a protein that is present in the cell. The resulting cDNA fragments may be size selected to obtain a population of a preferred size ranging from 30 to 1000 nucleotides. The preferred library is normalized to remove highly redundant sequences and increase the probability of selecting protein domains encoded by rare mRNAs. For example, normalization could be performed using subtractive hybridization to remove repetitive sequences (high Cot), (see e.g., Bonaldo et al., Genome Res. 6:791-806, 1996). An alternative to random cDNA synthesis is to make a library of DNAse fragmented cDNAs (from a library or from individual cDNAs) or using methods described by Roninson for the generation of GSEs (Gudkov et al., Proc. Natl. Acad. Sci. USA 91:3744-3748, 1994) and others (Fehrsen and du Plessis, Immunotechnology 4:175-184, 1999; Petersen et al., Mol. Gen. Genet 249:425-431, 1995; Kay (supra)).

Following vector construction and phage production, the phage cDNA library is contacted with cells, tissues or organs that display the receptor for which the cognate ligand is sought, and the ligand is selected using the selection strategies described herein. The cell line may express a natural receptor or be engineered to overexpress a recombinant receptor gene that is stabley expressed in that cell line using standard recombinant DNA methods. At 72 to 96 hours after phage addition, the cells are harvested and the reporter gene (e.g. GFP) positive cells are collected. The sequences encoding the ligand are recovered by PCR, rolling circle DNA amplification, or Hirt supernatant extraction, or the phage are reconstituted and used as input phage for the next round or selection. The library is monitored at each round of selection by restriction enzyme analysis of phage DNA to determine if the complexity of the library has been sufficiently reduced to allow identification of one or more active ligands. The ligand encoding sequences are compared to databases of known genes to determine if the recovered sequence is a portion of a known gene. Alternatively, the ligand encoding cDNAs are used as probes of conventional cDNA libraries to identify the full-length gene using standard molecular biology protocols for identification of full-length cDNA from partial sequences.

The feasibility of the aforementioned approach can be evidenced by using the cDNA encoding a proopiomelanocortin (POMC) gene (which encodes six different peptide ligands) that is fragmented at random by DNase 1, ligated to appropriate linker adaptors (see Chapter 9 by Du Plessis and Jordaan in Kay et al., 1998), and inserted into an appropriate phage or phagemid. The resulting library contains a mixture of DNA fragments, some of which will be inserted in-frame (⅓ if cloning is directional and ⅙ if cloning is bi-directional) with the pIII coat protein of the phage vector. The phage that display fragments encoding one of the six peptide ligands or a functional fragment thereof will bind and internalize in cells that display the appropriate receptor. The library is selected against cell lines that naturally express one of the receptors or against cells that are engineered to overexpress the receptor. For example, the POMC fragmented gene library is selected against COS cells that are made to overexpress the melanocortin1 receptor. Accordingly, the cDNA fragments that are selected encode the MSH-∝ peptide contained within the POMC cDNA. A series of overlapping cDNAs selected in this manner will define the minimal sequence that is sufficient to functionally interact with the melanocortin1 receptor. Thus, the minimal functional peptide is defined in a manner analogous to the identification of minimal epitopes for antibody binding using methods described by Geyson et al. (J. Immunol. Meth. 102:259-274, 1987). Similarly, the library may be selected against COS cells that overexpress ACTH or β-endorphin receptor to identify cDNAs encoding their cognate receptors.

In further embodiments, due to post-translational modifications, the selection of natural ligands for mammalian cells may be enhanced by utilizing ligands produced in mammalian cells and conjugating such ligands to ligand display packages of interest. Briefly, cDNAs are expressed in mammalian cells, thereby allowing post-translational processing and modification that takes place in these cells. In this regard, the phage may be conjugated to the mammalian cell synthesized library by non-covalent (e.g., electrostatic, etc.) linkage (e.g., an antibody-antigen epitope interaction, streptavidin-biotin, etc.) or by covalent means. The total cDNA is subcloned into a mammalian expression vector such as pSecTag2 (available from Invitrogen, CA), which is designed to tag each cDNA gene product with a binding site for an antibody or other site specific protein-binding sequence (e.g., IgG binding domain). As many cDNA molecules may contain stop codons, an additional tag may be added downstream of the secretion signal, but upstream of the cDNA insert (e.g., Flag, HA etc.). The cells are transfected with an epitope tagged library and distributed into individual or pools of transfected cells. The conditioned media is removed from the cells, phage that display an epitope binding antibody fragment or other epitope binding moiety are added to the medium (at about 10¹¹ pfu/ml), and the mixture is added to fresh target cells. The phage bind the ligands via the displayed antibody and the epitope tag on the ligand. Those ligands that are internalized by the target cells direct phage-mediated transduction of the target cells. Positive transduction is detected by GFP fluorescence, drug selection, or any reasonable detection means. The cells that secrete functional ligands are identified as those from which transduction competent conditioned medium was drawn and the positive cDNAs are recovered by PCR. In the case of pools of cDNAs, the pools are deconvoluted to identify individual cDNAs.

In yet additional embodiments, tissue-specific or tumor-specific ligands can be selected by pre-absorption of the phage library against normal or non-targeted tissues of cell cultures. For example, the selection process can be applied in vivo by injecting the library into an animal such as a tumor-bearing mouse. In such an experiment the tumor is removed from the mouse 48-72 h after injection. A cell suspension is prepared and phage genome bearing cells are selected by one of the methods described herein. The gene whose product allows entry and expression of the phage genome is then isolated from the drug resistant cell colonies.

Screening may be performed directly against target cells with no pre-screening or pre-enrichment. In one aspect, the present invention provides a method of identifying target cells or tissues for known or putative ligands. In this regard, phage display may be used to display a library of known or putative ligands (e.g., peptides, antibody fragments, and the like) and screen a singular tissue or cell type, or pools of tissues or cell types, thereby identifying target cells or tissues that are effectively transduced by a ligand. As used herein, “pool” refers to two or more cell or tissue types. In one embodiment, known ligands that are presented on a ligand displaying genetic package are contacted with a pool of a variety of cell or tissue types and, subsequently, the transgene expression is monitored. In a further embodiment, putative ligands are used to screen a pool of a variety of cell or tissue types for transduction ability. In this regard, ligands may be recovered and identified which efficiently transduce a particular tissue or cell type. Identification of cell specific ligands could greatly improve existing vectors for therapeutic gene delivery by targeting specific cells, thus reducing toxicity and allowing vectors to be administered systemically.

Such cell type or tissue-type screening provides for selection that requires biological interaction rather than simple binding, but does not require recovery of any infective phage. In addition, cell surface receptors need not be identified and purified for the screening to be effective. A further aspect of the present invention is that it can be easily adapted to high throughput applications for screening a variety of cell types or tissues and/or for screening libraries of putative ligands against libraries of putative receptors/binding partners (i.e., anti-ligands) which lead to transgene expression (see infra). In this regard, screening of a variety of ligand/cell interactions could be performed, including, for example, pathogen/host interactions, ligand/receptor, etc.

In one aspect, the present invention may be utilized to identify a variety of protein-protein interactions. In particular, a set of unknown proteins/peptides may be selected based upon interaction with another set of known or unknown proteins/peptides (e.g., random peptides, cDNA libraries, or antibody gene libraries). In one embodiment, putative ligands are displayed on the surfaces of filamentous phage that carry a reporter gene. These display phage are contacted with a cell line displaying a putative anti-ligand (protein/peptide) on its surface as a receptor fusion protein, such that the successful detection of the reporter gene requires binding of the phage display ligand and the cell surface displayed anti-ligand, as well as internalization and transgene expression. Such screening can be utilized in a variety of methods. For example, a known ligand may be screened against a library of potential anti-ligands. A library of unknown ligands may be screened against a known protein/peptide anti-ligand, and two libraries of peptides/proteins may be screened against each other to identify ligand/anti-ligand interactions (protein-protein).

A ligand/anti-ligand pair refers to a complementary/anti-complementary set of molecules that demonstrate specific binding, generally of relatively high affinity. Exemplary ligand/anti-ligand pairs include an antibody and its ligand as well as ligand/receptor binding. It should be understood that the designation of either component of the above mentioned ligand/anti-ligand pairs as either a ligand or anti-ligand is arbitrary. When necessary to specify a particular component, a “ligand”, as used herein, is meant to describe peptides or proteins displayed on a genetic package carrying an expressible transgene. Further, when necessary to define anti-ligand with specificity, an “anti-ligand”, as used herein, demonstrates high affinity and is expressed on the surface of the target cell to be monitored for transgene expression.

Any cell surface receptor may be used as a fusion construct for a cell surface displayed anti-ligand. However, in a preferred embodiment, the extracellular domain of the receptor is replaced with the putative anti-ligand. Construction of such fusions is routine in the art given that sequences, as well as the extracellular intracellular domains, of numerous receptors are known and available in the art Komesli et al., Eur. J. Biochem 254(3):505-513, 1998; Naranda et al., Proc. Natl. Acad. Sci. USA 94(21):11692-11697, 1997; Rutledge et al., J. Biol. Chem. 266(31):21125-21130, 1991; Lemmon et al., Embo J. 16(2):281-294, 1997; Foehr et al., Immunol. Cell Biol 76(5):406-413, 1998. Exemplary fusion constructs include, for example, anti-ligand-FGF receptor or anti-ligand-EGF receptor constructs.

In a further embodiment, a large pool of cDNAs may be tested by transfecting into a large number of mammalian cells (e.g., COS cells). Ligand displaying phage are exposed to the transfected cells, and positive cells are identified by either drug selection or detection of an expressed transgene (e.g., GFP sorted by FACs). PCR may be performed on single cells to identify ligand/anti-ligand binding pairs. In this regard, PCR primers directed to the known portion of the fusion construct may be used. For example, for phage display using pIII to display the ligand, the PCR primer will be directed to the pIII gene, while in order to identify the anti-ligand, the PCR primer will be directed to the surface membrane protein (e.g., a receptor domain) encoding portion of the fusion construct. Alternatively, the plasmids within positive cells may be rescued by Hirt supernatant method and separated from phage DNA by gel electrophoresis or chromatography. (Kay et al., supra). The selected cDNA plasmids may then be used to retransform bacteria. New plasmid DNA is prepared and used for additional rounds of screening by transfection into the cells and phage contact.

In an alternative embodiment, detection may be by any means which allows for the detection of the internalized nucleic acid molecules, and may include Hirt extraction of small DNA, direct polymerase chain reaction (PCR) amplification of ligand DNA from reporter gene expressing cells, other DNA amplification methods (e.g., phi 29 polymerase as well as other polymerases that function by rolling circle replication) and non-endogenous protein-nucleic acid molecule binding interactions (e.g., lac operon and lac repressor in a mammalian cell) in positive cells. In addition, it is possible to use direct PCR amplification, full vector PCR, RT-PCR, or rolling circle DNA amplification of ligand DNA from cells wherein no reporter gene is used, thereby allowing for direct identification of internalizing sequences in a high throughput fashion. These sequences could then be further characterized for the ability to deliver expressible genes or used to facilitate internalization of small molecules or polypeptides or like molecules conjugated or fused thereto.

In the various embodiments of the present invention utilizing PCR amplification of ligand sequences, the methodologies allow for the rapid amplification of only internalized sequences. Typical phage display technologies require that the phage of interest (e.g., that which binds to a particular target) be eluted and amplified following transduction of the appropriate host bacterial strain. However, transduction of bacteria requires that bacteriophage are intact and maintain infectivity. To eliminate the requirement for infective phage, methodologies provided herein allow those of ordinary skill in the art to recover, by PCR or other nucleic acid amplification methods, phage DNA sequences that have been internalized or internalized and trafficked to the nucleus, digest these sequences with appropriate restriction enzymes, remove extraneous sequences, subclone the desired sequences back into the phage display vector, and transform bacteria with this vector. Accordingly, by not requiring that the recovered phage be infective, the ability to display larger ligands as fusion constructs with coat proteins is possible. Further, recovery of uncoated phage, such as those targeted to the lysosomal or endosomal compartments, as well as those capable of directing expression in the nucleus is possible.

Briefly, in one aspect, the recovery by PCR amplification proceeds as follows: An initial selection of cells is performed using the detection of a reporter gene, selective conditions, or the like, and then the total DNA is recovered from these cells. The recovered DNA is used as a template for PCR primers that are designed to flank the sequence of interest (the ligand encoding sequence, Le., by using the pIII or pVIII sequences flanking the ligand insert as primer templates). The primers can be manufactured such that they can be easily removed from the ligand insert following restriction enzyme digestion, for example, the primers may contain a biotin moiety at the 5′-end. In the alternative, the primers may be removed by any known methods, including, for example, gel extraction, selective precipitation, and the like. In other alternatives, primers need not be removed, however, their removal facilitates ligation efficiency of ligand insert to vector. Further, the primers can provide restriction sites for subcloning.

Following amplification, the PCR product is purified to remove the polymerase and digested with restriction enzyme to excise the putative ligand insert. Enzymes are chosen in order to facilitate directional subcloning into either the original vector or a new construct, if so desired. Following enzyme digestion, the extraneous sequences are removed (e.g., by using biotinylated primers and streptavidin conjugated to beads or other solid support). The resulting DNA sequences are then ligated into a desired vector and the resulting vector is transformed into bacteria using standard methodologies. The transformed bacteria are then used to generate new phage particles or new DNA for additional rounds of screening. However, while it should be understood that the use of a reporter gene may lead to enhanced DNA recovery and fewer rounds of screening, there is not always a requirement that a reporter gene be used.

In an alternative embodiment, recovery of replicated internalized nucleic acid molecules may be achieved via a nucleic acid binding domain. Accordingly, when using phage, the phage genome can be altered such that a DNA binding sequence is incorporated therein. In one example, the phage vector may contain one or more copies of a lac operon, thereby allowing any internalized and replicated phage vectors to be purified from a cell lysate by a solid surface having conjugated thereto the lac repressor protein. Briefly, target cells are contacted with ligand displaying genetic packages (e.g., phage) for 48 to 72 hours. Since only the packages displaying an appropriate ligand are internalized and reach the cell nucleus where vector replication takes place, these will be the sequences that will be selected for and, thus, no reporter gene is required (e.g., GFP). Accordingly, the replicated vector is double stranded, and the double stranded form of the lac operon will bind the lac repressor. The cells are then lysed, and nuclear extracts are prepared, which are then passed over a solid support (e.g., Sepharose 4B) having conjugated thereto the lac repressor protein. The column is washed, then eluted by a salt or pH gradient, thereby releasing the bound DNA which can now be utilized in PCR reactions to amplify the ligand sequences for sub-cloning into another vector for further rounds of infection or characterization or the DNA can be used directly (without PCR) to transform bacteria and thereby produce more phage for further screening.

In other embodiments, the ligand displaying genetic package may also contain the nucleic acid sequences that encode the nucleic acid binding protein. For example, in the illustration above, the vector could also encode the lac repressor and the solid support has an anti-lac repressor antibody conjugated thereto, thereby allowing for recovery of nucleic acid molecules bound by the lac repressor.

One of ordinary skill in the art would readily recognize that a variety of nucleic acid binding proteins could be utilized as described above. In this regard, many proteins have been identified that bind specific sequences of DNA. These proteins are responsible for genome replication, transcription, and repair of damaged DNA. The transcription factors regulate gene expression and are a diverse group of proteins. These factors are especially well suited for purposes of the subject invention because of their sequence-specific recognition. Host transcription factors have been grouped into seven well-established classes based upon the structural motif used for recognition. The major families include helix-turn-helix (HTH) proteins, homeodomains, zinc finger proteins, steroid receptors, leucine zipper proteins, the helix-loop-helix (HLH) proteins, and β-sheets. Other classes or subclasses may eventually be delineated as more factors are discovered and defined. Proteins from those classes or proteins that do not fit within one of these classes but bind nucleic acid in a sequence-specific manner, such as SV40 T antigen and p53 may also be used.

These families of transcription factors are generally well-known (see GenBank; Pabo and Sauer, Ann. Rev. Biochem. 61:1053-1095, 1992; and references below). Many of these factors are cloned and the precise DNA-binding region delineated in certain instances. When the sequence of the DNA-binding domain is known, a gene encoding it may be synthesized if the region is short. Alternatively, the genes may be cloned from the host genomic libraries or from cDNA libraries using oligonucleotides as probes or from genomic DNA or cDNA by polymerase chain reaction methods. Such methods may be found in Sambrook et al., supra.

The helix-turn-helix proteins include well studied λ Cro protein, λcl, and E. coli CAP proteins (see Steitz et al., Proc. Natl. Acad. Sci. USA 79:3097-3100, 1982; Ohlendorf et al., J. Mol. Biol. 169:757-769, 1983). In addition, the lac repressor (Kaptein et al., J. Mol. Biol. 182:179-182, 1985) and Trp repressor (Scheritz et al., Nature 317:782-786, 1985) belong to this family. Members of the homeodomain family include Drosophila protein Antennapaedia (Qian et al., Cell. 59:573-580, 1989) and yeast MATα2 (Wolberger et al., Cell. 67:517-528, 1991). The zinc finger proteins include TFIIIA (Miller et al., EMBO J. 4:1609-1614, 1985), Sp-1, zif 268, and many others (see generally Krizek et al., J. Am. Chem. Soc. 113:4518-4523, 1991). The steroid receptor proteins include receptors for steroid hormones, retinoids, vitamin D, thyroid hormones, as well as other compounds. Specific examples include retinoic acid, knirps, progesterone, androgen, glucocosteroid and estrogen receptor proteins. The leucine zipper family was defined by a heptad repeat of leucines over a region of 30 to 40 residues. Specific members of this family include C/EBP, c-fos, c-jun, GCN4, sis-A, and CREB (see generally O'Shea et al., Science 254:539-544, 1991). The proteins of the helix-loop-helix (HLH) family appear to have some similarities to the leucine zipper family. Well-known members of this family include myoD (Weintraub et al., Science 251:761-766, 1991), c-myc, and AP-2 (Williams and Tijan, Science 251:1067-1071, 1991). The β-sheet family uses an antiparallel β-sheet for DNA binding, rather than the more common α-helix. The family contains MetJ (Phillips, Curr. Opin. Struc. Biol. 1:89-98, 1991), Arc (Breg et al., Nature 346:586-589, 1990) and Mnt repressors. In addition, other motifs are used for DNA binding, such as the cysteine-rich motif in yeast GAL4 repressor, and the GATA factor. Viruses also contain gene products that bind specific sequences. One of the most-studied such viral genes is the rev gene from HIV. The rev gene product binds a sequence called RRE (rev responsive element) found in the env gene. Other proteins or peptides that bind DNA may be discovered on the basis of sequence similarity to the known classes or functionally by selection. Furthermore, those of ordinary skill in the art will appreciate that the nucleic acid binding domain chosen for a particular recovery method will preferably be one which is not already present within the target cells.

In one embodiment, the LIVE™ method of identifying internalizing phage display ligands is based on the use of a genetic marker to tag the cells that have internalized phage. For example, GFP expression can be used to isolate cells that express a phage encoded GFP gene and FAC sorting used to select the GFP positive cells. The ligand encoding sequences are then recovered by PCR or other nucleic acid amplification methods and analyzed or used to make a second library of ligand display phage that is used for the next round of selection. The efficiency of selection at each round may be limited by the presence of a high ratio of phage DNA in the cytoplasm compared to the nucleus or the presence of a vast majority of phage remaining single-stranded following transfection of cells with a targeted phage, e.g., an EGF targeted phage, even in cells expressing the transgene, e.g. GFP. Purification of nucleic may be performed to reduce the amount of single-stranded phage DNA, but it does not completely eliminate it.

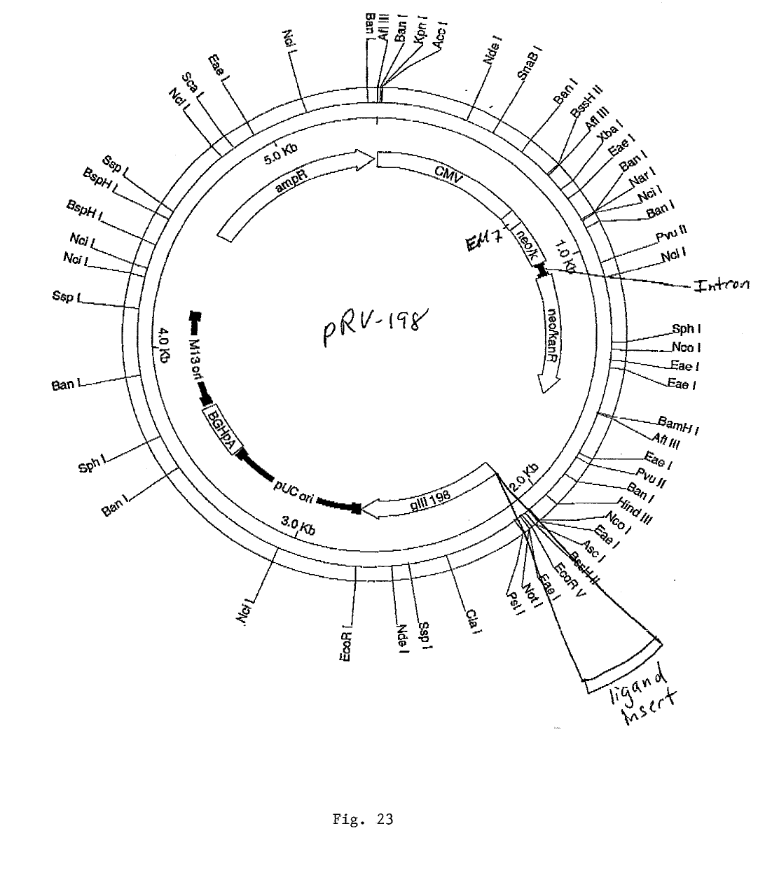

In certain embodiments, the invention provides a method for identifying phage that can translocate to the nucleus and for distinguishing between cytoplasmic internalized phage genomes and nuclear phage genomes that are capable of gene expression. This method utilizes a “retro-vector.” One example of a retro-vector is the phagemid vector pRV-198, shown in FIG. 23. The retro-vector typically contains a marker gene interrupted by intervening sequences, such as an intron, for example. The marker gene may be any gene capable of encoding an mRNA or polypeptide that may be identified or selected when expressed in cells containing the marker gene. For example, the marker may be a drug resistance gene capable of conferring drug resistance to cells expressing the drug resistance gene. Alternatively, the marker gene may be a nucleotide sequence capable of expressing an mRNA that can be identified by other means, such as RT-PCR, for example. The interrupted marker gene lies downstream of both a bacterial promoter sequence and a mammalian promoter sequence, each capable of transcribing the interrupted marker gene in suitable cells.

In mammalian cells, RNA processing enzymes can remove the intervening sequence from the interrupted marker gene to produce a transcript capable of expressing an uninterrupted marker gene mRNA or polypeptide. Thus, in certain embodiments, the retro-vector produces an RNA in a mammalian cell that when made into a cDNA (using reverse transcriptase, for example) and circularized by self-ligation becomes a functional plasmid capable of expressing the marker gene in bacterial cells. The mRNA produced by such a retro-vector contain a marker gene that is controlled by both a mammalian and bacterial transcriptional promoter, ligand encoding sequence (so that the ligand that allowed internalization and/or translocation to the nucleus may be identified), and a bacterial origin of replication. In one embodiment of the invention, the marker gene is a drug resistance gene capable of conferring drug resistance to a host bacterial cell following mRNA expression and processing in a transfected mammalian cell and subsequent isolation and transfer to a bacterial cell. In certain embodiments, the marker gene is the neomycin resistance gene, which can be selected for in bacteria using media containing the drug kanamycin, for example.

In another embodiment, a marker gene capable of expression in a transfected mammalian cell may be subsequently identified in a bacterial cell since mRNA expressed and processed in a mammalian cell will be shorter in length than unprocessed marker mRNA expressed in a bacterial cell. The different size messages can be determined by routine methods of mRNA amplification or detection, such as RT-PCR, for example. In a related embodiment, the absence or presence of the intervening sequence or intron in the marker gene may be detected in a bacterial cell following transfection and isolation from a mammalian cell, using routine methods known in the art, such as PCR or RT-PCR. For example, one or more primers may correspond to interveining sequence, so that amplification will only occur in the presence of interveining sequence. The absence of the intervening sequence indicates that the mRNA was expressed and processed in a mammalian cell, thereby permitting the identification and selection of phage or other ligand-displaying genetic packages or delivery vehicles capable of facilitating nuclear translocation and expression of a delivered nucleic acid sequence.

In certain embodiments of the retro-vector method, phage is produced using the retro-vector phagemid and contacted with suitable mammalian cells. After approximately 72-96 hours, the cells are washed to remove bound phage and lysed. Total mRNA is prepared from the lysed cells using standard protocols. RT-PCR is used to amplify the phage encoded mRNA using reverse transcirptase and oligonucleotides primers that bind specifically to the message to produce a cDNA that includes the bacterial promoter, the marker gene (e.g. drug resistance gene), the ligand fusion gene, and the bacterial origin of replication. The primers may have extended sequences that create restriction enzyme sites at each end of the cDNA. The ends of the amplified linear cDNA are cut with an appropriate restriction endonuclease and ligated to each other using T4 DNA ligase, for example, to form closed circular DNA. The closed circular DNA is used to transform E. coli bacteria and the drug resistant transformants are selected by growing on the appropriate drug-containing selective media. Only cDNA amplified from RNA will confer drug resistance. If single-stranded or double-stranded DNA is inadvertently amplified, it will not confer drug resistance because the drug resistance gene in the DNA vector is interrupted by an intron. Thus, the DNA must be transcribed in a mammalian cell and the intervening sequence (intron) removed by normal RNA processing enzymes in the cell to create a drug resistnace gene that will function in E. coli or other bacteria. Thus, the method is highly selective for only those phage genomes that have undergone transcription in a mammalian cell.

In a further embodiment, known or putative ligand-display phage may be used to screen a panel of cells that each express a potential target receptor. The source of the target receptor may be a known (i.e. cloned) receptor cDNA, or a collection of putative receptor cDNAs. For example, the putative receptor cDNAs may be identified from an epitope-tagged cDNA library as cDNAs that encode proteins that appear on the surface of cells. (see, Sloan et al., Protein Expression and Purification 11:119-124, 1997). Such cDNAs are inserted into an appropriate mammalian expression vector and transfected into a host cell. Preferably the host cell is eukaryotic, and more preferably the host cell is mammalian. The expression of the cDNA may be either stable or transient. Following expression, the cells are contacted with the ligand-display phage and monitored for transgene expression (e.g., drug resistance, GFP, or other detectable product). One skilled in the art would recognize that identification of cell or tissue types, as described above, in addition to using ligand display phage, could be also performed by utilizing other ligand displaying means, such as RNA-peptide fusions as described by Roberts and Szostak (Proc. Nat. Acad. Sci. USA 94:12297-12302, 1997), other phage types, viruses, or on bacteria.

Pre-screening or pre-enrichment may be used and can be especially helpful when either too few or too many hits are observed. Enrichment for cell binding may improve detectability if no hits are found in an initial screen. A prescreen to remove phage that bind non-specific cell surface proteins may reduce non-specific hits if there are too many initial hits. For example, infection of 1⁷ target cells is performed with about 10¹¹ phage, however a variety of cell density and phage titer ranges are useful. The cells are incubated for at least 2 hours and preferably 24-48 hours in PBSIBSA and washed extensively (Barry et al., Nature Med. 2:299-305, 1996). The cells are incubated in media at 37° C. for 24-96 hours and then detected or selected on the basis of expression of a reporter gene.

Assays for each of these reporter gene products are well known. For example, GFP is detected by fluorescence microscopy or flow cytometry. SEAP is detected in medium using a fluorescent substrate (Clontech; Palo Alto, Calif.). Human growth hormone may be detected in medium by a simple and sensitive radioimmune assay (Nichols Institute; CA). Western blotting and ELISA may also be used to immunologically detect and measure the presence of a reporter gene product. Alternatively, the message for a reporter gene is detected using RNase probe protection or fluorescent probe hybridization. For isolation of the phage vector DNA and insert, any technique that can identify and isolate the cells expressing detectable marker product may be used. Flow cytometry, in particular, is well suited for detecting fluorescence in or on a cell and isolating that cell. Further, flow cytometry is well suited for high throughput methodologies when necessary to isolate individual cells or groups of cells that express a reporter gene.

When the reporter gene is a selectable marker, cells are grown in selective conditions. Depending upon the marker, the conditions may be a particular growth temperature, addition of a drug, or the like. In the examples provided herein, the selectable marker is neomycin transferase, which confers G418 resistance on mammalian cells. Briefly, the cells are grown in the presence of G418 for 7-14 days or until resistant colonies are visible microscopically. Colonies are picked and phage vector DNAs recovered conveniently by amplification of the insert or a Hirt supernatent.

Alternatively, multiple rounds of infection and selection are performed to reduce the complexity of the infecting phage. For example, drug-resistant colonies are pooled and the selected inserts amplified and cloned back into the phage display vector for a new round of infection. When the reporter is fluorescent, flow cytometry can be used to select the strongest fluorescing cells to select the most highly efficient gene delivery ligands. More stringent screening conditions also include higher selective drug concentrations. At the completion of a selection process, representative phage clones may be subjected to DNA sequence analysis to further characterize gene delivery ligands.

In a further aspect, high throughput screening methodologies, such as screening libraries by sub-selection of pools, may be utilized to identify ligands. Briefly, phage stocks containing a variety of members, as individual plaques, may be used in combination with an array to identify potential internalizing ligands. For example, a stock of bacteriophage containing library members may be divided into subset pool stocks such that each stock contains about 10² to about 10³ members. Each stock solution is then screened utilizing an array (e.g., multi-well plates containing target cells). Upon detection of a reporter gene the phage stock may be sub-divided again and screened repeatedly until the phage which contains the internalizing ligand is identified. Alternatively, those of skill in the art will appreciate that the array may contain a variety of cell types which are capable of being screened with one or more phage libraries, of which may also include a variety of reporter genes (if so desired). Thus, multiplex screening is particularly applicable to the present invention. For example, a variety of alternatively colored fluorescent protein expression vectors are available that can be used as reporter genes to provide multiplexing capability (Clontech, Palo Alto, Calif.). Accordingly, rapid identification of those cells which internalize the bacteriophage and/or libraries that contain internalizing ligands for a specific cell type, may be identified. Utilizing both a variety of bacteriophage libraries, as well as a variety of cell types, would allow for a high throughput method of determining subsets of libraries that contain ligands for specific cell types simultaneously. Arrays for binding biomolecules are known in the art and therefore could be adapted to utilize the phage screening methodology of the present invention, see, e.g., PCT Application No. WO 95/11755, PCT Application No. WO 95/35505, U.S. Pat. No. 4,591,570. In addition, affinity based biosensors such as a Biacore instrument, available commercially from Biacore AB, Uppsula, Sweden, may be used to immobilize phage or cells for high throughput screening.

Moreover, while commonly used high throughput methodologies that utilize live cells are typically performed on arrays of 6 to 96 well plates, the current invention may also be carried out using cellular micro-arrays, such as those described by U.S. Pat. No. 5,776,748. Briefly, such arrays may be manufactured such that designated areas of the array bind a defined number of cells or size of tissue. For example, the arrays can be constructed such that they bind only a single cell. Therefore, an array of single cells may be constructed with a variety of cell or tissue types. Because the size of the cell binding islands on the array may be chosen such that no more than one cell may bind on any given island, because the locations and geometric pattern of the islands may be predetermined, and because the cells will remain at fixed locations during assaying, cellular micro-arrays can provide for a high efficiency and high throughput method of assaying for internalizing ligands, anti-ligands, or target cells or tissues.

In a preferred embodiment, flow cytometry is utilized, and the cells are identified and counted by an automated detector unit. Because the locations and geometric patterns of the islands are predetermined, the detector can be designed or programmed to take measurements specifically at those locations. Therefore, identification of individual cells that have been successfully transduced by a ligand displaying genetic package carrying a nucleic acid molecule that encodes a detectable product is easily accomplished. In some embodiments, cells transduced by a ligand displaying genetic package carrying a nucleic acid molecule that encodes a selectable marker may be first selected on the basis of the appropriate sensitivity or resistance and then plated as individual cells and further selected or characterized by the methods described herein. In particular, selection may be employed prior to plating on the plates to isolate transformed or transfected cells, and then the cells may be assayed in situ.

In addition, when using fluorescence assays, a detector unit may be placed above the plate or, if the plate is translucent, below the plate. In the case of transmission spectrophotometric assays, a translucent plate is used, a source of electromagnetic radiation is placed on one side of the plate and a detector unit on the other. Because of the small distances between individual isolated cells permitted by the present invention, detectors employing fiber optics are particularly preferred. Such sources of electromagnetic radiation and such detectors for electromagnetic transmission, reflection or emission are known in the applicable art and are readily adaptable for use with the invention disclosed herein.

The constructs and methods disclosed herein are also applicable to screening in vivo. Such screening may be performed similar to methods for targeting organs or xenograft tumors using phage displayed peptides (Pasqualini et al., Nature Biotech. 15: 542-546, 1997; Pasqualini et al., Nature 380: 364-366, 1996; and U.S. Pat. Nos. 5,622,699; 6,232,287; and 6,068,829, all of which are incorporated by reference herein in their entirety), except that the tissues, organs, or tumors themselves, and not just vasculature, are examined for reporter gene expression or internalization of the ligand bearing genetic package, instead of merely the presence of infective phage in the vasculature of that tissue. Briefly, a phage display library is delivered to an animal, generally mice, but preferably humans, either intravenously, through an airway, intrathecally, through the digestive tract, or any other method of delivery, and organs or tumor samples are generally tested for reporter gene function or phage DNA recovery at about 48-96 hours to about 1, 2, or 3 weeks, including all incremental integer values of time therebetween, following delivery. Tumor cells may be cultured in selective conditions or sorted by flow cytometry or any other method to enrich for cells that express the phage transducing gene. The ligand encoding sequences can be amplified from selected cells as described above. As in in vitro screening, repeated rounds of infection and re-screening, alone or in combination with increased screening stringency, may be used to obtain the most efficient gene delivery ligands.

In one embodiment, applicable to in vivo or in vitro screening, the present invention utilizes nucleic acid amplification to detect and/or characterize internalizing ligand bearing genetic packages. This method has been termed “LIVE-NA”. The LIVE-NA method is particularly valuable for identifying ligands that target and internalize in cells of different organs and tissues from libraries that are applied to whole animals.

The existing technology for selecting peptide display ligands is referred to as in vivo biopanning. In vivo biopanning is a phage display method that is used to select peptides from highly diverse phage display libraries that “home” to various organs. In the biopanning method, the libraries are injected into an animal and typically allowed to circulate briefly (several minutes), after which the animal is sacrificed, and homing phage are recovered from the target organ by contacting an extract of the organ with bacterial cells. Surviving infectious phage are thus propagated in the host bacteria to achieve an amplification of selected peptide display phage. The peptides that are isolated by this technique have been shown to bind unique molecular addresses in the vasculature of the target tissue (such as a specific peptidase). Thus, in vivo panning appears to be largely limited to finding binding peptides that are trapped in vasculature of target organs. See, e.g., Molenaar et al, Virology 293: 182-191, 2002.

The present invention provides a more useful selection for the purpose of drug and gene delivery, since it allows the identification of peptides that target cells within the organ itself and not simply the vasculature, likely due to extravasation of genetic packages. In vivo panning generally fails to identify these sorts of peptides at least in part because: 1) phage penetration into tissues, while possible, is a slower process and therefore longer time periods (hours to days) may be needed for targeted phage to accumulate in the targeted organ or tissue; and 2) those phage that have penetrated into the organ and internalized into cells are likely to have lost infectivity due to proteolysis and, therefore, are not retrieved by in vivo biopanning. However, the present invention overcomes these problems by not requiring infective phage recovery.

One embodiment of the selection strategy, termed LIVE-NA, is based on extraction of the phage DNA or RNA (that encodes the targeting ligand) from the targeted tissue or organ. Identification of cells that have internalized phage may be accomplished through the use of a reporter gene encoded by the phage. However, direct recovery of the phage DNA/RNA can also be accomplished by extraction of circular phage DNA (or transcribed RNA) from the organ/tissue, nucleic acid amplification (by PCR or single temperature amplification with a phage polymerase), and transformation of suitable host bacteria with the DNA using electroporation. Extensive washing in acid and or protease solution is used to remove externally bound phage and phage that are trapped in the vasculature. The selected phage library can then be characterized by sequencing individual phage DNA or used as input for the next selection round.

Accordingly, in various embodiments, the LIVE procedure does not require that the selected phage be infective and, therefore, is more likely to recover internalized phage that have been degraded. Furthermore, the phage library can be allowed to circulate for longer time periods, such as hours or days, to allow penetration onto desired target organs without regard to phage protein degradation. In fact, phage DNA internalized by cultured cells remain remarkably intact over a period of at least three days following internalization. In addition, even if some phage DNA degradation has occurred, DNA amplification would be useful for recovering the remaining phage that are intact.

One of ordinary skill in the art would recognize that amplification could then be extended to phage encoded RNA to recover ligand encoding sequences from cells in organs that have internalized phage and expressed the encoded product. For example, a promoter (e.g., CMV) may be placed upstream of the ligand encoding sequences in the phage genome and a transcriptional termination signal (e.g., polyadenylation site) placed downstream. Phage that internalize and traffic to the nucleus will transcribe RNA encoding the ligand sequence. The transcription of RNA typically amplifies the DNA sequence 10 to 100-fold. RT-PCR (cDNA synthesis with reverse transcriptase followed by PCR) may be used to obtain further amplification. The ligand encoding sequences may then be characterized by sequencing and/or subcloned back into the phage vector and used for the next selection round.

Specificity may also be examined in vitro using a panel of non-targeted and targeted cell lines and detecting expression of the phage transducing gene. Competition studies with a free ligand or a neutralizing antibody to the ligand or receptor are used to confirm specific entry of phage via the ligand receptor complex. Alternatively, the cloned receptor for the ligand can be overexpressed in a cell line that normally does not express that receptor. Phage internalization and expression into the stable transfectants expressing the receptor, but not the parent cell line, indicates the specificity of the ligand for its receptor on receptor bearing cells.

Ligands that are identified as gene targeting ligands using the selection strategies described herein may be further tested for specificity by reporter gene expression in target and non-target cells and tissues. The ligand may also be tested in a variety of gene delivery methods, such as ligand-polylysine/DNA complexes (Sosnowski et al., J. Biol. Chem. 272:33647-33653, 1996) or retargeted adenovirus gene delivery (Goldman et al., Cancer Research 57:1447-1451, 1997).

The specificity of the targeting ligand may alternatively be determined in vivo by biodistribution analysis using one of the reporter genes described herein, such as luciferase. At various time points, mice injected with the ligand displaying phage are sacrificed and tissues examined for the presence of phage in non-targeted tissues by immunohistochemistry, an enzymatic assay that detects reporter product activity, or the like.

III. Uses

The methods described herein are designed to select cDNAs, Fabs, sFv, random peptides, and the like for discovery of new ligands or anti-ligands. The methods can also be used to select mutated and gene-shuffled versions of known ligands for targeting ability. Accordingly, as discussed above, the methodologies described herein allow for the directed evolution of genetic packages and/or ligands to develop ligands or whole vectors that deliver their associated components to the interior of the cell and facilitate expression of associated nucleic acid molecules. Thus, the methods can be used to identify ligands useful for delivery of any molecule to the interior of a cell and can be further screened to target specific intracellular compartments, such as nucleus, mitochondria, chloroplast, etc. The methods also can be used to direct the evolution of genetic packages into a vector for gene delivery and confer mammalian-cell specific tropism. Thus, the design is selected for a function as opposed to a biased engineering approach.

Although it is possible to modify vectors both chemically and genetically for more efficient gene transfer, the choice of each enhancing element must be determined by trial and error. However, with genetic display packages such as phage, it becomes possible to apply the power of phage display and genetic selection to the evolution of more efficient vectors and thus bypass the more tedious and time-consuming process of rational design. Indeed, along these lines, it is demonstrated herein that it is possible to selectively enrich for specific phage by their ability to introduce a reporter gene into target cells. Thus, novel sequences, unanticipated by rational design, can be selected from libraries of highly diverse peptides or cDNAs using the methods described herein.

This directed evolution can also be used to create phage that are more suitable for in-vivo gene delivery having, for example, increased serum half-life, selective tissue targeting, and/or decreased immunogenicity. A recent example of this is the selection of long-lived phage by repeated rounds of injection of phage libraries into animals and selection of surviving phage using either lambda or T7 phage. See, e.g, Merril et al., Proc. Natl. Acad. Sci USA 93(8):3188-3192, 1996 and Sokoloff et al., Mol. Ther 2(2):131-139, 2000. Sokoloff et al. have identified sequences that increase the half-life of T7 phage by protecting phage against complement activation. Perhaps even more importantly, these peptides could be used as “stealthing” agents to protect other gene or drug delivery vectors from clearance.

The work of Pasqualini and coworkers also demonstrates the ability to evolve phage in-vivo for the ability to home to the vasculature of specific tissues or to tumors. Recently, Samoylova et al. applied in vivo panning to identify phage that targeted muscle, indicating that phage can be developed that penetrate the vasculature to target tissues in vivo. Muscle Nerve 22(4):46-466, 1999. Accordingly, these cited studies demonstrate that the disclosed methodologies utilizing the present invention allow not only evolving of a targeting agent, but more importantly, an agent that targets, internalizes, and facilitates the expression of an associated nucleic acid molecule. Thus, it allows for the evolution of a highly effective vector for in vivo gene delivery, rather than creation through rational design.

An advantage of the methods of the present invention, however, is that they permit the targeting or introduction of nucleic acids or other molecules associated with a display package into selected tissue- or organ-specific cells of target tissues or organs, and not just into the vasculature of target tissues or organs. Similarly, the methods of the present invention permit the identification of ligands that bind and internalize into tissue-specific cells of specific tissues and organs, rather than merely vasculature-associated cells of target tissues and organs. Accordingly, the invention permits the targeting of substantially non-vasculature-associated cells of a target tissue or organ and the identification of ligands that facilitate binding and/or internalization into substantially non-vasculature cells of a specific tissue or organ. In certain embodiments, the substantially non-vasculature cells are substantially non-endothelial cells.

A related advantage of the present invention is that it permits the targeting or introduction of nucleic acids or other molecules associated with a display package to the parenchyma or parenchymal cells of a tissue or organ. As used herein, the parenchyma is the specific tissue or an organ as distinguished from its supportive, vasculature, or connective tissue, for example. Similarly, the methods of the present invention permit the identification of ligands that bind and internalize into the parenchyma or parenchymal cells of a tissue or organ.

Relatedly, the methods of the present invention permit detection of internalization events at various time points following transfection of a mammalian cell with a phage or other ligand displaying genetic package. For example, detection may be performed at least 96 hours following transfection, thereby allowing extra time for the phage or ligand displaying genetic package to target tissue-specific cells, as opposed to vasculature-associated cells. Without being bound to any particular theory, it is hypothesized that the additional time permits the phage or ligand-displaying genetic package to extravasate from the vasculature into the parenchyma or tissue-specific cells. Of course, detection may also be performed at any other time following transfection, as described previously, including, for example, up to 24 hours, 24 to 48 hours, 48 to 72 hours, 72-96 hours, and all integer values between. Methods of detection that may be performed at these different time points include all those described herein, including the detection of gene expression by a selectable marker or direct mRNA expression.

These ligands may have increased transduction efficiency (as measured by an increase in the percentage of infected cells that express the reporter gene), increased expression of the reporter gene (as measured by intensity of reporter gene expression) in the phage transduced cells, increased specificity of transduction for target cells (as measured for ligand specificity), increased stability of the ligand (as measured by ability to target the ligand in vivo to tumor cells), increased affinity for receptor (e.g., removing dimerization requirements for ligands that dimerize), increased functionality (e.g., stimulates internalization), elimination of the need for cofactors (e.g., development of an FGF variant that binds with high affinity to the FGF receptor but not to heparin), and altered specificity for receptor subtypes (e.g., an FGF variant that reacts with only one of the four FGF receptors).

The ligands identified by the methods described herein may be used as targeting agents for delivering therapeutic agents to cells or tissues. For example, a therapeutic gene can be incorporated into the phage genome and delivered to cells via phage bearing the gene delivery ligand on its protein coat.

A transducing gene, as used herein, refers to a gene that encodes a detectable product in a target cell. Preferentially, the transducing gene is a therapeutic gene. A “therapeutic nucleic acid” or “therapeutic gene” describes any nucleic acid molecule used in the context of the invention that effects a treatment, generally by modifying gene transcription, translation, or supplies a replacement or amplification of an existing gene. It includes, but is not limited to, the following types of nucleic acids: nucleic acids encoding a protein, ribozyme, antisense nucleic acid, DNA intended to form triplex molecules, protein binding nucleic acids, and small nucleotide molecules. As such, the product of the therapeutic gene may be DNA or RNA. These gene sequences may be naturally-derived sequences or recombinantly derived. A therapeutic nucleic acid may be used to effect genetic therapy by serving as a replacement for a defective gene, by encoding a therapeutic product, such as TNF, or by encoding a cytotoxic molecule, especially an enzyme. The therapeutic nucleic acid may encode all or a portion of a gene, and may function by recombining with DNA already present in a cell, thereby replacing a defective portion of a gene. It may also encode a portion of a protein and exert its effect by virtue of co-suppression of a gene product.

As discussed above, the therapeutic gene is provided in operative linkage with a selected promoter, and optionally in operative linkage with other elements that participate in transcription, translation, localization, stability and the like.

The therapeutic nucleotide composition of the present invention is from about 20 base pairs to about 100,000 base pairs in length. Preferably, the nucleic acid molecule is from about 50 base pairs to about 50,000 base pairs in length. More preferably, the nucleic acid molecule is from about 50 base pairs to about 10,000 base pairs in length. Even more preferably, it is a nucleic acid molecule from about 50 pairs to about 4,000 base pairs in length.

In one embodiment, therapeutic nucleic acids used according to the invention are ribozymes. A ribozyme is an RNA molecule that specifically cleaves RNA substrates, such as mRNA, resulting in specific inhibition or interference with cellular gene expression. Generally, a ribozyme is an RNA that has both a catalytic domain and a sequence homologous to a particular mRNA. The ribozyme functions by associating with the mRNA (through the homologous domain of the ribozyme) and then cleaving (degrading) the message (using the catalytic domain). There are at least five known classes of ribozymes involved in the cleavage and/or ligation of RNA chains. Ribozymes can be targeted to any RNA transcript and can catalytically cleave such transcripts (see, e.g., U.S. Pat. Nos. 5,272,262; 5,144,019; and U.S. Pat. Nos. 5,168,053, 5,180,818, 5,116,742 and 5,093,246 to Cech et al.). Methods of designing and using ribozymes are known in the art, and are described, for example, in the aforementioned patents, as well as U.S. Pat. Nos. 5,334,711, 5,225,337, 5,625,047, 5,631,359, 6,022,962, and references cited within.

In another embodiment, therapeutic nucleic acids used according to the invention are antisense molecules. Antisense molecules are oligonucleotides that bind in a sequence-specific manner to nucleic acids, such as mRNA or DNA. Antisense RNA technology involves expressing or introducing an RNA molecule (or derivative) that is homologous to sequences found in a particular mRNA into a cell. By associating with the mRNA, the antisense RNA inhibits use of the mRNA for production of the protein product of the gene. (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucl. Acids Res. 21:3405-3411, which describes dumbbell antisense oligonucleotides). Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988)). Methods of designing and producing antisense molecules to disrupt expression through a particular sequence element are well known in the art.

Other therapeutic agents that may be delivered using targeting agents and ligands identified by the methods of the invention include small molecules, such as organic molecules and synthetic and designed small molecules, for example. Such small molecules may be delivered according to the invention to stimulate or inhibit the function of a wide variety of intracellular targets and to treat a large number of diseases. A variety of therapeutic small molecules are known in the art, and methods of designing, optimizing, synthesizing, and manufacturing small molecules are also known to those of ordinary skill in the art.

The ligands/anti-ligands provided herein are useful in the treatment and prevention of various diseases, syndromes, and hyperproliferative disorders, such as restenosis, other smooth muscle cell diseases, tumors, such as melanomas, ovarian cancers, neuroblastomas, pterygii, secondary lens clouding, and the like. As used herein, “treatment” means any manner in which the symptoms of a condition, disorder or disease are ameliorated or otherwise beneficially altered. Treatment also encompasses any pharmaceutical use of the compositions herein. As used herein, “amelioration” of the symptoms of a particular disorder refers to any lessening, whether permanent or temporary, lasting or transient, that can be attributed to or associated with administration of the composition.

In certain embodiments, the compositions of the present invention may be used to treat angiogenesis-dependent diseases. In these diseases, vascular growth is excessive or allows unwanted growth of other tissues by providing blood supply. These diseases include angiofibroma, arteriovenous malformations, arthritis, atherosclerotic plaques, corneal graft neovascularization, delayed wound healing, diabetic retinopathy, granulations due to burns, hemangiomas, hemophilic joints, hypertrophic scars, neovascular glaucoma, nonunion fractures, Osler-weber syndrome, psoriasis, pyogenic granuloma, retrolental fibroplasia, scleroderma, solid tumors, trachoma, and vascular adhesions.

By inhibiting vessel formation (angiogenesis), unwanted growth may be slowed or halted, thus ameliorating the disease. In a normal vessel, a single layer of endothelial cells lines the lumen, and growth of the vessel requires proliferation of endothelial cells and smooth muscle cells.

As well, the ligands, anti-ligands, and cells identified by the present invention may be used to treat tumors. In these diseases, cell growth is excessive or uncontrolled. Tumors suitable for treatment within the context of this invention include, but are not limited to, breast tumors, gliomas, melanomas, prostate cancer, hepatomas, sarcomas, lymphomas, leukemias, ovarian tumors, thymomas, nephromas, pancreatic cancer, colon cancer, head and neck cancer, stomach cancer, lung cancer, mesotheliomas, myeloma, neuroblastoma, retinoblastoma, cervical cancer, uterine cancer, and squamous cell carcinoma of skin. For such treatments, ligands are chosen to bind to cell surface receptors that are generally preferentially expressed in tumors.

Through delivery of the compositions of the present invention, unwanted growth of cells may be slowed or halted, thus ameliorating the disease. The methods utilized herein specifically target and kill or halt proliferation of tumor cells having receptors for the ligand on their surfaces.

The identified ligands/anti-ligands may also be used to treat or prevent atherosclerosis and stenosis, a process and the resulting condition that occurs following angioplasty in which the arteries become reclogged. Generally, treatment of atherosclerosis involves widening a stenotic vascular lumen, permitting greater blood flow and oxygenation to the distal tissue. Unfortunately, these procedures induce a normal wound healing response in the vasculature that results in restenosis. Of the three components to the normal vascular response to injury, thrombosis, elastic recoil and smooth muscle cell proliferation, anti-thrombotics/platelet inhibitors and vascular stents effectively address acute/subacute thrombosis and elastic recoil, respectively. However, no existing therapy can modify the vascular remodeling that is due to proliferation of smooth muscle cells at the lesion, their deposition of extracellular matrix and the subsequent formation of a neointima. Accordingly, phage could be used to deliver therapeutic nucleic acids or polypeptides that would inhibit restenosis.

Wound response also occurs after other interventions, such as balloon angioplasty of coronary and peripheral vessels, with or without stenting; carotid endarterectomies; vein grafts; and synthetic grafts in peripheral arteries and arteriovenous shunts. Although the time course of the wound response is not well defined, if the response can be suppressed for a short term (approximately two weeks), a long term benefit is achieved.

In certain aspects of the invention, the invention provides a method of selecting or identifying therapeutic molecules, such as nucleic acids or small molecules, for example. A ligand displaying package comprising a ligand capable of binding and being internalized into a cell suffering from a disease or disorder may be used to deliver a therapeutic molecule to the cell. Thus, the same ligand displaying package may be used to deliver candidate therapeutic compounds to a cell with a disease or disorder. The effect of delivering candidate therapeutic molecules to the cell may be determined at one or more suitable time points following delivery of a candidate therapeutic molecule to a cell. Any phenotypic cell characteristic, such as gene expression, proliferation, cell cycle, and attachment-dependence, for example, or any characteristic or symptom associated with a disease or disorder may be examined. One of ordinary skill in the art understands that such characteristics differ depending on the particular disease or disorder being examined, and appropriate characteristics associated with any disease are known to those of skill in the art. Therapeutic products may be identified by their ability to lessen a disease or disorder-related characteristic or symptom following delivery to the cell.

In a related embodiment, a therapeutic product may be identified by a method comprising identifying an internalizing ligand for a cell using any method of the invention, using a ligand displaying package comprising the identified ligand to deliver a candidate therapeutic molecule, such as a nucleic acid or small molecule, for example, to a diseased cell as described above, and identifying a therapeutic molecule from one or more candidates. The cell may be a cell suffering from a disease or it may the same type of cell as a diseased cell, for example.

In another related embodiment, a therapeutic molecule or drug may be manufactured or produced by identifying an internalizing ligand for a diseased cell, identifying a therapeutic molecule for the disease, and manufacturing or producing ligand displaying genetic packages comprising the identified ligand and the identified therapeutic molecule.

In other various embodiments, the applications of the technology described herein is virtually limitless. For example, bacteriophage may be retargeted (e.g., redirected from their native binding) using ligands added to their coat to treat bacterial disease in plants and animals. Briefly, ligands may be screened against a variety of pathogenic bacteria and ligands which effectively deliver expressible products to the interior of the cell may be utilized to shuttle toxic components selectively into these bacteria. One of ordinary skill in the art would readily recognize that the methods described herein may be modified to alter the native binding of a bacteriophage such that the bacteriophage binds and injects its contents or is internalized, thereby delivering its contents in select bacteria. Accordingly, once ligands are identified, the bacteriophage may be used to delivery cytotoxic expression plasmids to the interior of the bacteria or, alternatively, the ligands may be attached to toxic chemical moieties that will be delivered to the bacteria in high concentrations via the ligand targeting agent. Further, the mere delivery of a replication competent phage to a bacteria could induce death by cell lysis, thereby creating a bacteriophage antibiotic, specifically targeted to select bacteria. Such targeting may also be extended to targeting plant cells, yeast, fungi, and virtually any other cell or microorganism. For example, food production may be enhanced by transducing yeast or cheese producing bacteria with bacteriophage carrying a gene of interest and targeted to the yeast or bacteria using a ligand identified by the methods described herein.

The present invention provides the capability of identifying ligands which internalize as well as proteins, antibodies, cell/cell interacting proteins that define the interrelationships between cells, host/pathogen, tumor/stroma, autocrine/paracrine factors and allows identification of molecules that are targets for new drug discovery or are themselves therapeutically or diagnostically useful. Further, other peptides can be discovered by the methodologies taught herein that enhance endosomal escape, nuclear localization, cell binding, and thus discover ligands that are useful not only in phage mediated gene therapy, but generally applicable to standard gene therapy methodologies (e.g., enhancing gene expression from animal viral vectors, DNA-conjugates etc.)

The following examples are offered by way of illustration, and not by way of limitation.

### EXAMPLES

### Example 1

### Modified Phage Vectors for Mammalian Cell Transduction

A mammalian expression cassette is inserted into a phage or phagemid vector and is used to detect ligand mediated phage entry via reporter gene expression in mammalian cells. A type 3 filamentous phage vector is modified for transduction of mammalian cells by insertion of a GFP expression cassette consisting of a CMV mammalian transcriptional promoter, the green fluorescent protein gene from pEGFP-N1 (Clontech; Palo Alto, Calif.), and a bovine growth hormone transcriptional terminator and polyadenylation signal to make the vector, MEGFP3 (see FIG. 1A). The mammalian expression cassette also contains an SV40 origin of replication adjacent to the CMV promoter. Similar constructs for monitoring entry and subsequent expression of phage genomes in mammalian cells are constructed from other known phage or phagemid vectors including pCANTAB 5 E (Pharmacia Biotech; Piscataway, N.J.) or M13 type 3 or 33 for gene III fusions (see, Kay et al., Phage Display of Peptides and Proteins: A Laboratory Manual, Academic Press, 1996; McConnell et al., Mol. Divers. 1:165-176, 1996) and M13 type 8 or 88 vector for fusions to gene VIII protein (Roberts et al., Methods Enzymol. 267:68-82, 1996; Markland et al., Gene 109:13-19, 1991).

### Example 2

### Construction of FGF2-containing Phage Display Vectors

In the following examples, a phage that displays FGF2 on its surface is used to bind to the FGF2 receptor on mammalian cells and be internalized. An FGF2 gene is subcloned into the modified M13 phage type 3 vector, MEGFP3, to create the ligand display phage, MF2/1G3 (see FIG. 1B). The gene may also be mutated such that it encodes an FGF2 (C96S) (C78S) double mutant which enhances expression efficiency. The MEGFP3 vector has been modified with a mammalian expression cassette designed to express the reporter gene GFP to monitor mammalian cell transduction by the phage. Other vectors include pCANTAB 5 E (Pharmacia Biotech; Piscataway, N.J.) or M13 type 3 or 33 for gene III fusions (see, Kay et al., Phage Display of Peptides and Proteins: A Laboratory Manual, Academic Press, 1996; McConnell et al., Mol. Divers. 1:165-176, 1996). Similarly, FGF2 is cloned into M13 type 8 or 88 vector for fusion to gene VIII protein (Roberts et al., Methods Enzymol. 267:68-82, 1996; Markland et al., Gene 109:13-19, 1991).

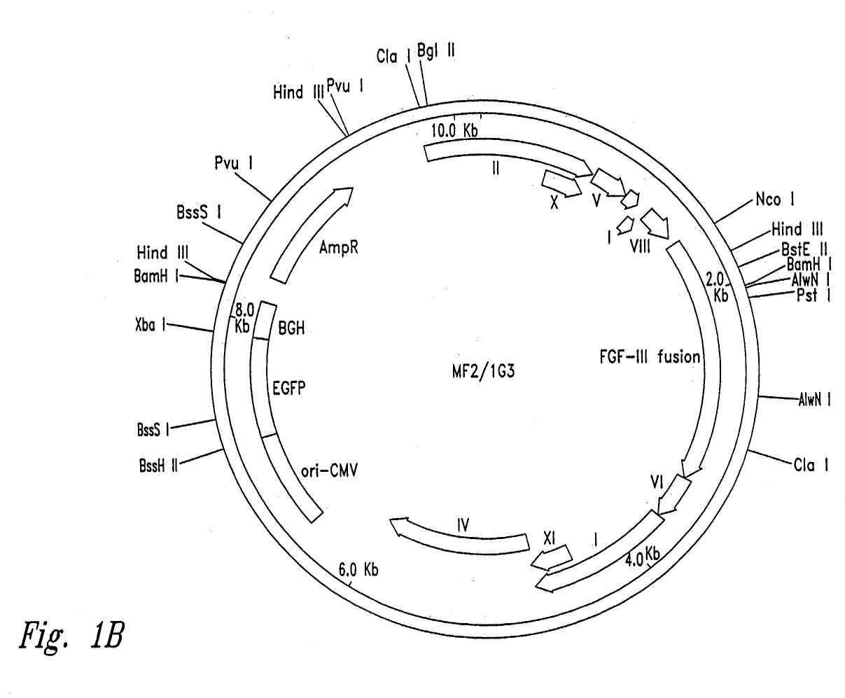

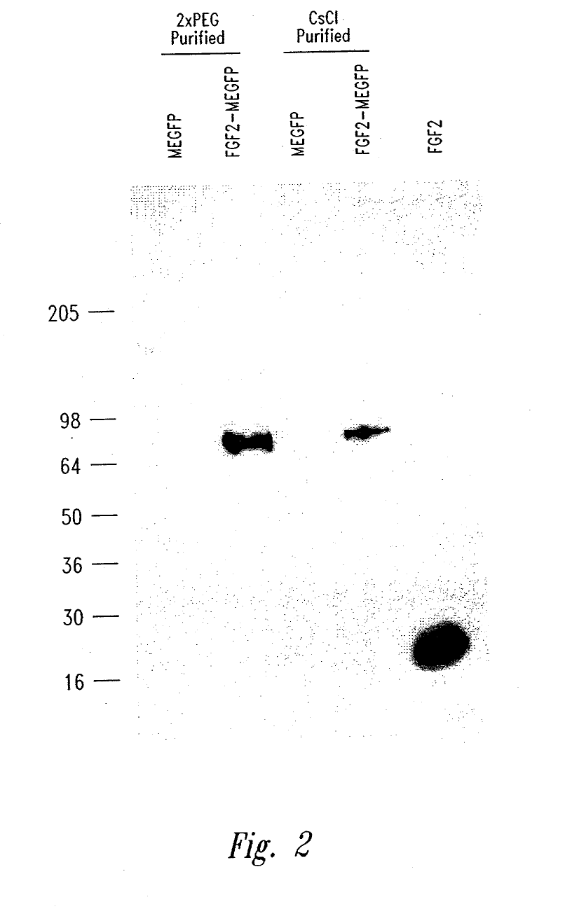

To facilitate cloning, the FGF2 gene is amplified by PCR using oligonucleotide primers that contain appropriate restriction endonuclease sites in the phage vector gene III or VIII genes. The resulting phage express FGF2 on their surface coat as detected by anti-FGF2 antibodies in Western blots (FIG. 2) and by ELISA (FIG. 3).

Western blot detection of FGF2-pIII fusion utilizes extracts from equivalent phage titers of purified FGF2 phage and control phage (MEGFP3) separated by polyacrylamide gel electrophoresis and blotted onto nitrocellulose. FGF2 and FGF2-fusion phage are detected with an anti-FGF2 monoclonal antibody (Transduction Labs; Lexington, Ky.) and HRP conjugated anti-mouse secondary antibody (American Qualex; San Clemente, Calif.) with chemiluminescent development. A single protein band is detected in the cesium chloride purified FGF2-phage extract migrating at about 80 kDa. This is about the size predicted for the FGF2-pIII fusion protein (FGF2 (18 kDa) fused to pIII (migrates ˜60 kDa)). CsCl purification is performed to remove any non-covalently bound FGF2 fusion protein from the phage particles.

Binding of the FGF2 fusion phage to FGF2 receptor is assessed by ELISA in which recombinant FGF2 receptor is attached to the solid phase and an anti-phage antibody is used as the primary detection antibody. Briefly, phage were captured with an anti-FGF2 rabbit polyclonal antiserum bound to the plate well. An HRP conjugated anti-M13 antibody (Pharmacia Biotech; Piscataway, N.J.) was used to detect the bound phage. When anti-phage antibody is used to capture the phage and equivalent OD is observed for both control (MEGFP3) and FGF2-phage (MF2/1G3) indicating that equivalent phage particles are applied to the plate (FIG. 3A). In FIG. 3B an increased OD indicates the presence of FGF2 on the MF2/1G3 FGF2-phage.

### Example 3

### Target Cell Line Engineering

To increase the sensitivity of the assay for transduction by ligand display phage the target cell line is transfected with a plasmid that is designed to express the SV40 large T-antigen (i.e. pSV3neo). This plasmid also contains a drug selection gene such as neomycin phosphotransferase (neo) which confers resistance to the antibiotic G418 in stabley transfected mammalian cells. Following transfection of the target cell line with plasmid DNA using standard methods (i.e. CaPO₄ co-precipitation) the cells are split and maintained in G418 containing media until drug resistant colonies appear. The colonies are expanded to test for SV40 T-antigen synthesis by western blotting or immunoprecipitation using a suitable antibody. Examples of T-antigen expressing target cell lines are: BOS (BHK with SV40 T-Ag) for screening FGF variants, HOS-116 (HCT116 with SV40 T-Ag) for screening peptides that target human colon carcinoma, and AOS-431 (A431 with SV40 T-Ag) for screening EGF variants (all parent cell lines are available from ATCC, Manassas, Va.).

### Example 4

### Binding and Internalization of FGF2-expressing Phage

The FGF2-expressing phage are also assayed for high affinity receptor binding and internalization in receptor bearing cells by immunolocalization and fluorescence microscopy (Hart, J. Biol. Chem. 269:12468-12474, 1994; Barry et al., Nature Med. 2:299-305, 1996; Li, Nature Biotech. 15:559-563, 1997).

Infection of mammalian cells with FGF2-expressing phage is performed under conditions that block entry of wild type M13 phage into cells except chloriquine is not used (Barry et al., supra). Phage are added directly to cells at titers of ≦10¹⁰ CFU/ml in PBS with 0.1% BSA or other suitable blocking agents and incubated at 37° C. or on ice for at least 1 hour. The cells are then washed extensively in PBS, fixed in 2% paraformaldehyde, and permeabilized in 100% methanol at room temperature for 10 minutes. Cells are incubated with rabbit anti-M13 antibody (Sigma; St. Louis, Mo.) in PBS/BSA for 1 hour. The primary antibody is detected with a phycoerythrin labeled anti-rabbit antibody (Life Technologies (Gibco BRL); Rockville, Md.). Surface bound (incubated on ice) or internalized (37° C. incubation) phage are detected by fluorescence microscopy.

### Example 5

### Transduction of Mammalian Cells by FGF2-ligand Display Phage

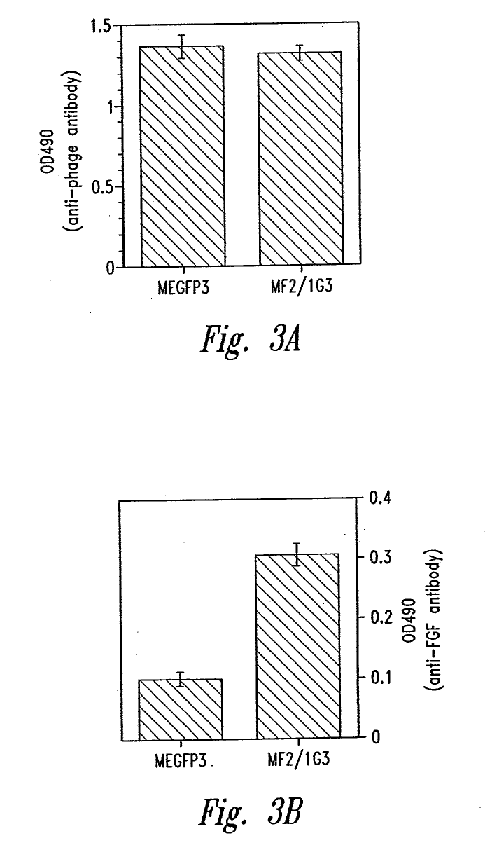

FGF2 display phage (MF2/1G3) and an identical phage that lacks the FGF2 gene (MEGFP3) are compared for receptor mediated internalization and reporter gene expression in COS cells. The phage are incubated with the cells for 4 hours at 37° C. in DME (Dulbecco's modified Eagles medium, Life Technologies (Gibco BRL); Rockville, Md.) containing 2% BSA (bovine serum albumin) as a blocking agent. After washing to remove unbound phage the cells are returned to the incubator for an additional 72 hours. Transduction is measured by counting GFP positive autofluorescent cells. As shown in FIG. 4B, the FGF2 display phage result in about a 10 fold greater transduction efficiency than the control phage, indicating that the displayed FGF2 ligand on the surface of the phage particles results in receptor mediated binding and internalization of phage with subsequent expression of the phage reporter gene. The specificity of the FGF2-phage mediated transduction is demonstrated by successful inhibition of transduction with excess free FGF2 (2 μg/ml) (FIG. 4B). The low level nonspecific uptake and transduction by the control phage (MEGFP3) is not affected by the presence of excess FGF2.

It is important to show that the MEGFP3 control phage is equally capable of transducing mammalian cells as the display phage when appropriately targeted. To compare the transduction ability of both the FGF2-phage and the control phage, equivalent titers of each phage were used to transfect COS cells using a avidin-biotin FGF2 targeting method. In this method biotinylated FGF2 is contacted with the cells and used to capture phage particles via the addition of avidin and a biotinylated anti-phage antibody. The phage/FGF2/cell binding is performed on ice, unbound phage removed by washing, cells returned to the incubator at 37° C., and transduction assessed at 72 hours. As seen in FIG. 4A, there is no significant difference in transduction between FGF2-phage and control phage when FGF2 is attached to the phage via an avidin biotin linkage. In this case the biotinylated FGF2 is in excess of the FGF2 displayed on the phage surface such that internalization is expected to be primarily via the biotinylated FGF2. These data demonstrate specific receptor mediated transduction of mammalian cells by filamentous phage that genetically display a targeting ligand (FGF2).

### Example 6

### Construction of a Reporter Gene and a Drug Resistance Gene in Phage Display Vectors

A GFP expression cassette consisting of the GFP gene (Cormack et al., Gene 173:33-37, 1996) under control of a CMV promoter, a neomycin phosphotransferase gene under control of the SV40 early gene promoter, and an SV40 origin of replication are cloned into a gene III phagemid vector such as PCANTAB 5E using standard methods (Sambrook et al., Molecular Cloning: A Laboratory Manual, Cold Spring Harbor Press, 1989). The resulting phage is designated pmaM13. The same phagemid genome also containing FGF2-3 fused to gene III is designated pFGF-maM13. Similar constructs are also made with M13 phage type3 and type 33 and gene VIII phagemid and phage vectors. Recombinant phage displaying FGF2 on the coat and carrying the mammalian expression cassettes including the SV40 replication origin are prepared by phagemid rescue with M13K07 (or suitable helper phage) are added to COS cells as described above. GFP expression is detected by fluorescence microscopy, fluorometry, and flow cytometry at 48-96 hours after phage addition. Drug resistant cells are selected with G418.

### Example 7

### Selection of FGF2-expressing Phage from a Mixed Population

A M13 phage display library of random or unknown sequences is spiked with pFGF-maM13 phage. The mixture is used to infect COS cells as described above. The cells are washed extensively to remove non-specifically bound phage. Cells are re-plated 48-96 hours later at a 1 to 10 dilution and grown in G418 to select only cells that receive the transducing phage gene. Alternatively, the GFP expressing cells are isolated by flow cytometry using an excitation wavelength of 488 and emission wavelength of 510.

DNA is extracted from G418-resistant cells and the FGF2 sequence is amplified. The amplification primers have sequences complementary to phage sequences located on each side of the FGF2 sequence in the gene III coding sequence. Detection of the FGF2 sequences in selected COS cells that are infected with a mixture of phage where the pFGF-maM13 phage is diluted at least 1:10,000 with the random sequence phage library demonstrates feasibility of the technique.

### Example 8

### Identification of FGF2 Variants for Improved Gene Delivery

A library of shuffled FGF2 mutants is created using the gene shuffling method described by Stemmer (supra). The FGF2 gene is amplified by PCR and fragmented by DNase 1 treatment. The fragments are reassembled using PCR in the absence of primers. The reassembled gene is cut with the appropriate restriction enzymes and cloned into an M13 phage vector such that the FGF mutants are fused in-frame with the pIII coat protein gene. The phage vector contains a CMV promoter driven GFP reporter gene and an SV40 origin of replication. Several individual phage clones are sequenced to confirm that an average of 3 mutations per phage have been generated during the reassembly process. The resulting phage library of FGF2 mutations is amplified by standard protocols. The target cell line, BOS (BHK with T-Ag) is incubated with the library such that each member of the library is at an m.o.i. of at least 10. Accordingly, 10¹¹ phage representing 10⁶ copies of 10⁵ individual phage species are applied to 10⁵ cells. The phage are incubated with the cells in PBS supplemented with 2% fetal bovine serum for 1-3 hours, after which non-binding phage are removed by extensive washing with PBS. Media is added and the cells returned to the incubator at 37° C. to allow phage internalization.

### Example 9

### Screening Libraries for Gene Delivery Ligands

If the source of the desired ligand is not known, random peptide libraries or a cDNA library from placenta is used as a starting point for cDNA library screening. The library is amplified in the maM13-33 phage by infecting DH5αF′ (or other suitable host) bacteria, growing the culture overnight at 37° C. and isolating the phage from the culture medium using standard protocols. A cDNA library containing 10⁵ members has each member represented 10⁶ times in a typical phage titer of 10¹¹ colony forming units/ml. The amount of phage used to infect is adjusted to the complexity of the library.

The completed maM13 phage library is screened against the target tissue or cell line. Screening can be performed in vitro or in vivo. The criteria for a positive “hit” is that the phage must be able to bind, be internalized, translocate to the nucleus, uncoat and replicate and express the genomic DNA containing the reporter gene in the target cell. Thus, only transduced target cells are selected either by GFP expression and cell sorting or drug resistance. Screening is performed directly against the target cells with no prescreening or enrichment. Enrichment for cell binding is performed if no hits are found in the initial screen. A prescreen to select out phage that bind non-specific cells surface proteins is performed to reduce non-specific hits or if there are too many initial hits. Infection of at least 10⁷ target cells is performed with at least 10¹¹ phage. The cells are incubated for at least 2 hours in PBS and washed extensively as described by Barry (Barry et al., Nature Med. 2:299-305, 1996). The cells are incubated in media at 37° C. for 48-96 hours and selected in the appropriate drug (e.g., G418) for 7-14 days or until resistant colonies are visible microscopically. Drug resistant colonies are pooled, and the selected cDNAs amplified and subcloned back into the maM13-33 phage vector using PCR and standard molecular biology methods. Alternatively individual colonies are screened. Representative phage clones are sequenced to identify potential gene delivery ligands. Repeated rounds of infection and selection are performed to reduce the complexity of the selected clones. More stringent screening conditions such as increased selective drug concentrations or FACS sorting or the strongest fluorescent cells are performed in the later screens to select the most highly efficient gene delivery ligands from the initial screening.

Screening in vivo is performed using methods similar to those described by Pasqualini for targeting organs or xenograft tumors using phage displayed peptides (Pasqualini, R. et al., Nature Biotechnology 15, 542-546, 1997; Pasqualini, R. et al., Nature 380, 364-366, 1996) except that the organs or tumors are examined for reporter gene expression instead of the presence of phage. The phage library may be delivered into an animal such as a mouse and organs or tumor samples tested for phage DNA or RNA or reporter gene function at 48-96 hours after delivery or for a time sufficient for phage penetration and internalization which could require screening at between several days to several weeks to allow the phage to exit the vasculature and penetrate the tissue itself for subsequent internalization. Tumor cells are cultured in G418 or FACs sorted (for GFP expression) to enrich for cells that express the phage transducing gene. The ligand encoding sequences are amplified from selected cells using PCR as described for in vitro screening. As in in vitro screening, repeated rounds of infection and rescreening are performed at increasing screening stringency to obtain the most efficient gene delivery ligands.

### Example 10

### Identification of Ligands that Target Colon Carcinoma

In this example, a library of oligonucleotides encoding random peptides is inserted into a filamentous phage genome such that the peptides are fused to the C-terminus of intact pIII coat proteins. A type 3 phage vector that only contains one copy of the pIII gene is used and, therefore, all of the pIII protein that is made will be fused to a peptide. Thus, 3-5 copies of a peptide is displayed on each phage. To simplify the screening the complexity of the library is first reduced by screening it for internalizing peptides. Peptides that facilitate the internalization of phage into a colon carcinoma cell line are isolated through several rounds of selection. The phage library is incubated with the cells for 3 hours at room temperature. The cells are washed extensively in PBS. A brief proteinase K treatment is used to inactivate phage that adhere to the cell surface. The cells are then lysed and cell lysates incubated with host bacteria. Internalized phage are amplified in bacteria and subjected to 4 or more iterations of exposure to cells and recovery of internalized phage. Replicative form DNA is prepared from the resulting sublibrary of internalizing phage. The random sequences in the sublibrary are subcloned into a phage vector MEGFP2 that contains a copy of the CMV driven reporter gene (GFP) and an SV40 replication origin. MEGFP2 differs from MEGFP3 (FIG. 1A) in that the ori-CMV/EGFP expression cassette is in the reverse order, EGFP is followed by an SV40 polyadenylation site instead of Bovine Growth Hormone poly A, and the vector contains three additional Nco I sites within the ori-CMV/EGFP expression cassette.

The resulting CMV-GFP modified sublibrary is incubated with the HOS-116 recipient cell line such that each member of the library is represented at least 10⁶ times. Thus, for example, a library with 10⁵ members is added to ˜10⁵ cells at a titres of ˜1×10¹¹ yielding an m.o.i. for each member of at least 10. The phage are incubated with the cells in PBS supplemented with 2% fet al bovine serum for 1-3 hours, after which non-binding phage are removed by extensive washing with PBS. Media is added and the cells returned to the incubator at 37° C. to allow phage internalization.

### Example11

### Recovery of Ligand Encoding Sequences from Replicative Phage

At 72 hours following the addition of the phage library, the target cells are removed from the plate and sorted for GFP expressing cells by FACS. The positively sorted cells are lysed and treated with proteinase K. The proteins are extracted with phenol/chloroform (24:1 solution) and nucleic acids precipitated in ethanol. The resulting DNA is resuspended in S1 nuclease buffer and treated with S1 nuclease to remove non-replicative single strand phage DNA. The DNA is again extracted with phenol/chloroform, precipitated, and resuspended in polymerase chain reaction buffer. Alternatively, nuclei are prepared from the positive cells, proteinase K treated and the lysate used directly in the PCR reaction. In either case, an equivalent number of negatively sorted cells are treated in parallel and used in the PCR reaction to monitor the enrichment of replicative phage DNA (double-stranded) over non-replicative phage DNA (single stranded) such that there is no phage DNA amplified in the samples from GFP negative cells. If phage DNA is amplified from negatively sorted cells then conditions must be made more stringent for the removal of single stranded phage DNA such as increasing treatment with S1 nuclease or further purification of nuclei through repeated sucrose step gradient purification or other suitable methods known for purification of nuclei (to remove non-replicative phage). These conditions might need to be determined empirically for each cell line and library used.

The phage sequence(s) encoding the ligand peptide is amplified using an appropriate set of oligonucleotide primers that flank the ligand encoding DNA sequence inserts that is fused to the pIII gene. These amplified inserts are recloned into the parent phage vector to create a sub-library of phage enriched now for gene delivery ligands for the target colon carcinoma cell line. Sequencing is performed on representative clones to determine the complexity. The screening process is reiterated until the complexity is reduced sufficiently to identify one or more targeting ligands.

### Example 12

### Second Generation Screening of Peptides

Peptides are selected which have been previously identified from a random library by one or more panning or screening procedures using conventional vectors and panning methods (see Kay et al., Phage Display of Peptides and Proteins: A Laboratory Manual, Academic Press, 1996). The DNA encoding the selected peptides is inserted as a fusion to the pIII coat protein in the MEGFP2 vector containing the GFP reporter gene cassette.

An M13 phage random peptide library is screened for peptides that bind and internalize in an FGF receptor overproducing cell line, Flg37 (an FGFR1 stable transfectant of L6 cells (available from the ATCC; Manassas, Va.) obtained from Dr. Murray Korc, UCl; Irvine, Calif.). In addition, such a cell line may be easily created by those skilled in the art. Following 5 rounds of panning and rescreening the complexity of the library is reduced such that 80% of the phage are represented by a single peptide-pIII fusion. The resulting peptide, FL5, has the sequence FVPDPYRKSR (SEQ ID NO: 1). The same library is also screened against Flg37 cells by selecting infective phage particles that internalize and associate with nuclei and cytoskelet al proteins. The 2 predominant peptide sequences identified by this screen after 5 rounds of panning are FN5A, CGGGPVAQRC (43%) (SEQ ID NO: 2) and FN5B, CLAHPHGQRC (34%) (SEQ ID NO: 3).

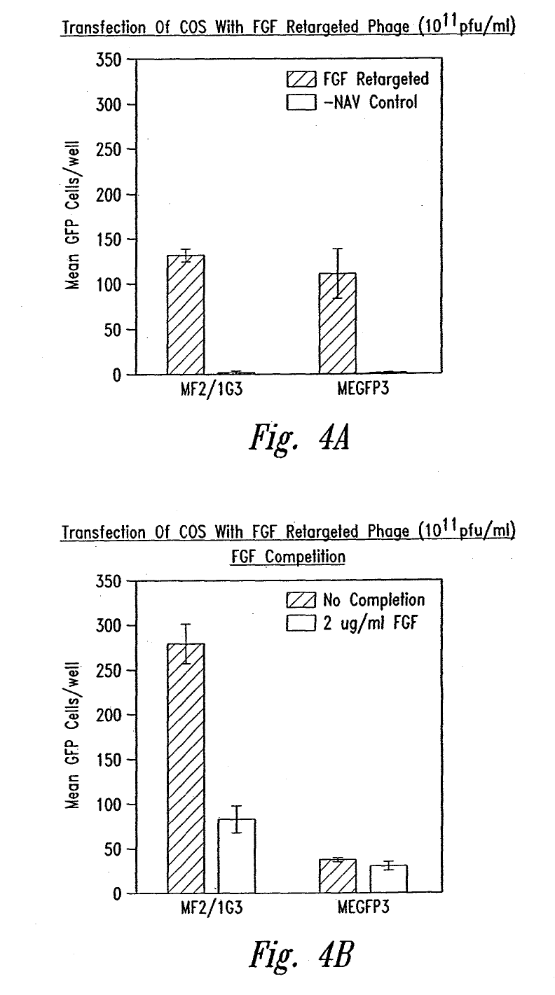

Oligonucleotides encoding the 3 peptides are inserted into the MEGFP vector as fusions to the pIII coat protein. The resulting phage are used to transfect COS cells. Phage are added to cells and incubated overnight at 37° C. in medium with 10% fet al calf serum. The cells are washed to remove unbound phage and returned to the incubator. Transduction is assessed by counting GFP expressing autofluorescent cells at 72 hours after the addition of phage. The results (FIG. 5 are that a greater transduction efficiency is observed with FL5 than FN5A or FN5B indicating that FL5 is more efficient as a gene transfer ligand in this system. The transduction screening method as a second generation screen is capable of distinguishing among peptides that were selected by different primary cell based screens.

### Example 13

### EGF Mediated Mammalian Cell Transduction

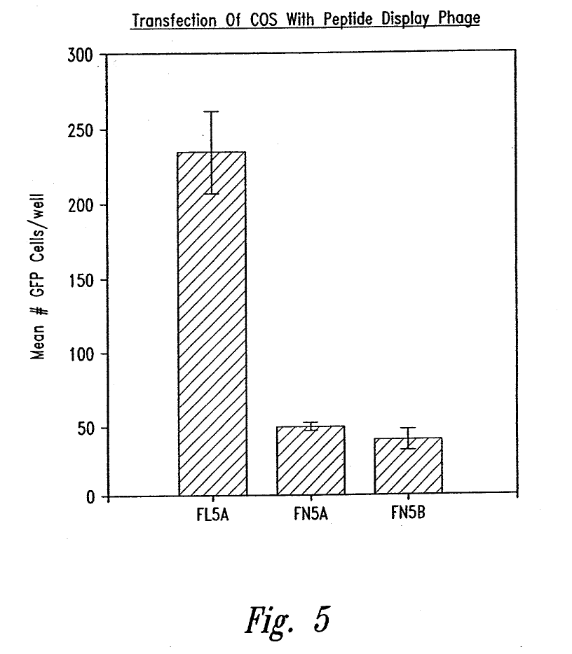

Epidermal growth factor displaying phage were constructed as described above for FGF displaying phage. Western blot analysis demonstrates that EGF was efficiently expressed on the phage coat in a multivalent manner (FIG. 6). Phage were prepared for Western analysis by obtaining the EGF-phage from cultures of infected host bacteria, and purified by PEG precipitation and CsCl gradient centrifugation. The phage particle proteins were then separated by gel electrophoresis and blotted onto a nitrocellulose membrane. Blots were then probed with either anti-EGF or anti-pIII antibody (mouse anti-human EGF, Biosource International; Camarillo, Calif.) or anti-pIII antibody (mouse anti-pIII, MoBiTech; Germany) followed by HRP-goat-anti-mouse (Jackson Laboratories, USA).

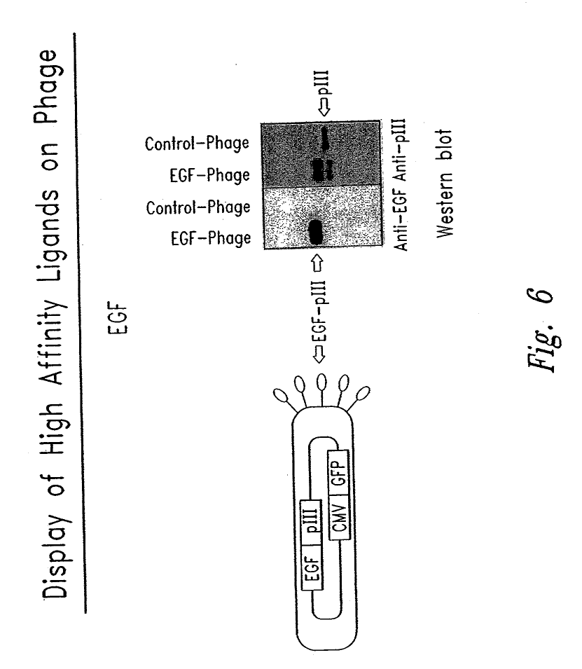

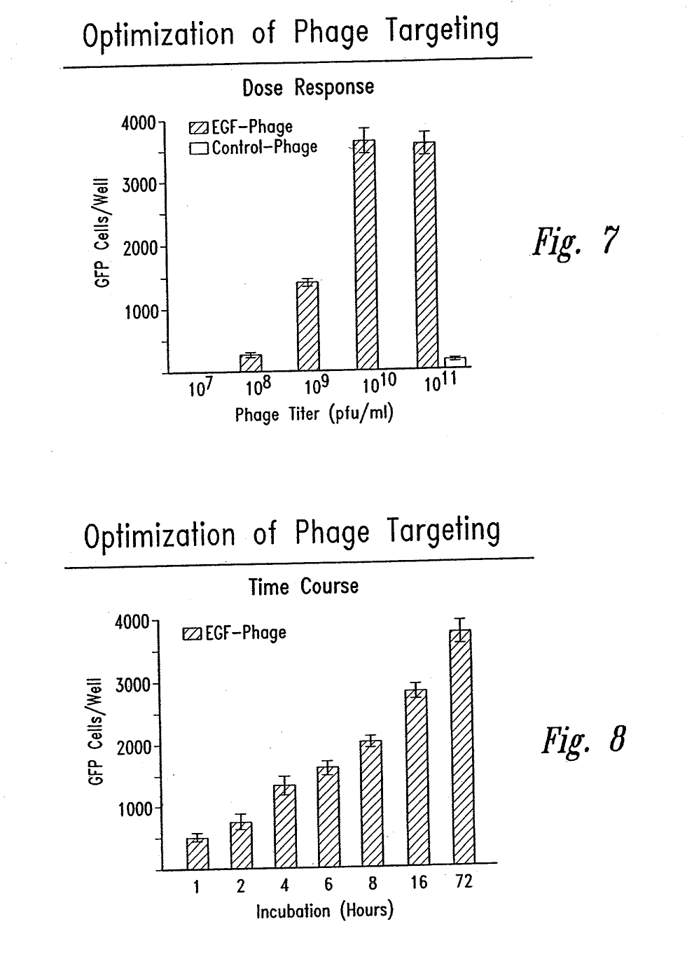

Following the procedures detailed above, EGF-phage were screened for their ability to effectively transduce COS cells. Briefly, EGF-phage were incubated with COS cells (˜75,000 cells/well) for 72 hours with a variety of phage titers. As demonstrated by FIG. 7 the optimal dose was 10¹⁰ pfu/ml which resulted in the highest transduction efficiency with almost no non-specific transduction by untargeted phage. Transduction efficiency also increases with longer incubation times. As demonstrated in FIG. 8, when EGF-phage were incubated with COS cells (˜75,000 cells/well) at 10¹¹ pfu/ml for various times and subsequently measured for GFP expression at 72 hours, longer incubation times increased transduction efficiency.

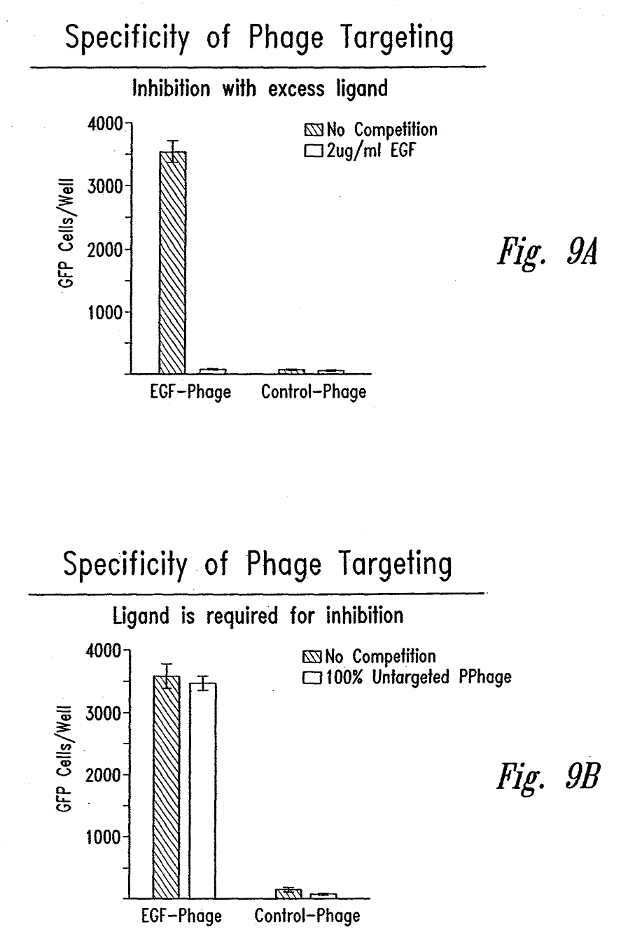

Further, specificity of EGF-phage mediated COS cell transduction was determined by incubating EGF-phage with excess ligands. As depicted in FIGS. 9A and 9B, COS cells incubated with 10¹¹ pfu/ml of phage for 72 hours with or without excess ligands or untargeted phage demonstrate that targeting is due to the presence of the ligand.

### Example 14

### Simultaneous Identification of Internalizing Ligands and Anti-ligand Binding Targets

To identify internalizing ligand-anti-ligand binding target interactions, the putative ligand is displayed on the surface of filamentous phage that carry a mammalian reporter gene expression cassette. The candidate binding target peptides/proteins are expressed on the surface of COS cells by substituting the target cDNA for the extra cellular domain encoding DNA portion of the EGF receptor in a suitable mammalian cell expression vector (i.e., pcDNA 3.1; Invitrogen, CA).

To accomplish this, a library of cDNAs is inserted into a mammalian expression vector (pcDNA 3.1) such that the cDNAs are fused to the transmembranes and intracellular domains of EFG receptor cDNA. DNA is prepared from individual or pools of bacterial clones that have been transformed to carry the cDNA-receptor fusion protein expression plasmid. COS cells are transfected with the resulting plasmid DNAs in six well plates at low density. At 24 hours later, ligand display phage carrying the CMV driven reporter gene GFP are added to the transfected COS cells.

Binding of the phage displayed ligand to the cell surface display binding target (i.e. protein—EGF receptor fusion protein), results in dimerization of the receptor and subsequent internalization of phage that displays the binding ligand. The internalized phage are trafficked to the nucleus where the reporter gene is expressed. 72 hours after adding phage, cells expressing the reporter gene are selected by FACs. cDNAs encoding reactive peptides are identified by the presence of GFP positive cells in the COS transfectants for each cDNA or cDNA pool. The binding ligand is identified by PCR amplification and sequencing of the phage ligand-pIII fusion gene. The target peptide is identified by PCR amplification and sequencing the peptide-EGF receptor fusion protein from the selected cell(s).

### Example 15

### Identification of Cell Targets

Phage that display a ligand as a pIII fusion on the phage coat and carry the GFP expression cassette are prepared using standard protocols, as discussed above. Control phage that carry GFP but don't display a ligand are also prepared. Candidate cell targets are seeded into 6 well culture plates at about 40,000 cells/well. At 24 hours after seeding the cells, phage are added at ˜10¹⁰ pfu/ml. The plates are incubated at 37° C. for an additional 72 hours. Each cell well is scored by counting GFP positive autofluorescent cells. The cell types that have a ratio of GFP positive cells in the ligand-phage treated well/control phage treated cells of greater than 1.0 are selected as targets for further study and characterization. As an alternative to GFP, a drug resistance gene can be used in which case after 72 hours the cells are allowed to continue growth in selective medium containing the drug. Positive cell types are scored by counting wells that have drug resistant colonies.

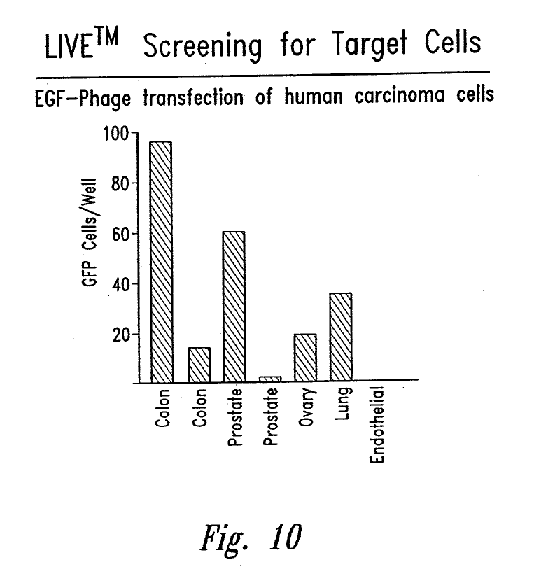

Carcinoma cell lines which are known to express EGF were screened by the above method using EGF-phage and compared to the control endothelial cell line which is EGF receptor negative (Cell lines obtained from ATCC, Manassas, Va. and grown under standard ATCC culture conditions). As shown in FIG. 10, the carcinoma cell lines derived from various tissues were differentially transduced while the receptor negative, endothelial cells displayed no transduction. Accordingly, identification of target cells or tissues can be accomplished using these methods.

### Example 16

### Identification of Pathogen Target Cells

Ligand display phage are constructed as discussed above, with the ligand being full-length or fragments of coat or envelope proteins of a known or suspected pathogen. The ligand expressed on the display phage coat can be expressed from the cDNA or cDNA derivative of the coat or envelope protein of a known or suspected pathogen (e.g., HIV envelope protein gene). The envelope gene is randomly fragmented to form a library of display phage display distinct portions of the coat protein, thereby allowing determination of the portion of the gene that encodes a protein that functionally interacts with the host cell surface receptors allowing internalization. Smaller pathogen coat proteins are displayed in entirety. The pathogen coat display phage acts as surrogate pathogen with the advantage of providing a simple assay for detection of host cells. Phage displaying coat protein are screened against various cell types in vitro as described above or in vivo by injection and subsequent identification of target cells and tissues by fluorescent microscopy, FACS analysis to detect GFP, or growth in selective medium to detect expression of a drug resistance marker.

### Example 17

### Identification of Pathogen Ligands

The gene(s) or portion of a gene that interacts with the host cell surface receptor to allow internalization is identified by making a phage display library of the cDNAs expressed by the pathogen or of the pathogen genome or fragments of the genome. The display library phage vector carries the GFP or suitable reporter gene driven by the mammalian promoter, as described above. The libraries are then screened against known or putative host cell types by detecting transgene expression (i.e., drug selection or other detectable marker). Once cells are identified, the sequence of the nucleic acid encoding the internalizing ligand is determined by PCR sequencing of the pIII-putative ligand fusion construct.

### Example 18

### Identification of Secreted and Internalizing Ligands for Tumor Cells

Tumor cells interact with surrounding host stromal and other cell types via chemo-attractants and other factors which, for example, stimulate the stromal cells to secrete factors that support tumor growth (i.e., VEGF). To investigate these interactions, a library of putative secreted ligand cDNAs is prepared from tumor cell mRNA and selected by methods known in the art such as epitope-tagging, Sloan et al., Protein Expression and Purification 11:119-124, 1997. The secreted proteins encoding cDNAs are inserted into the reporter phage vector as described above.

Individual phage clones or pools of phage clones are screened against various stromal cell types to identify cell types that are targets for tumor cell secreted factors, and to identify the secreted factors. The inverse strategy can also be applied by screening a library of, for example, fibroblast or other stromal cell secreted proteins encoding cDNAs for factors that bind and internalize into various tumor cell types.

### Example 19

### Selection of EGF-expressing Phage from a Population of Non-targeted Phage

Non-targeted M13 phage was spiked with EGF-phage. The mixture was used to infect COS cells and incubated for 72 hours, as described above. The cells are washed extensively to remove non-specifically bound phage. The GFP expressing cells are isolated by flow cytometry (FACS) using an excitation wavelength of 488 and emission wavelength of 510.

DNA was extracted from GFP positive cells and the EGF sequence was amplified by PCR. The amplification primers have sequences complementary to phage sequences located on each side of the EGF sequence in the gene III coding sequence. These sequences were re-cloned into the phage vector and new phage were prepared for subsequent rounds of selection.

Briefly, the following biotinylated oligonucleotides were used to amplify the ligand gene III fusion:

| | | | |
|---|---|---|---|
| Anchor1M8f | 48 5′Bio | AAAGGATCCGGGTTCCCGCGTGGGCGATGGTTGTTGTCATTGTCGGC | (SEQ ID NO: 5) |
| M3rev2 | 25 bio | CCGTAACACTGAGTTTCGTCACCAG | (SEQ ID NO: 6) |

However, the following have also been used with success:

(SEQ ID NO: 7)

| | | |
|---|---|---|
| M8for2b | 30 biotin | GCGTGGGCGATGGTTGTTGTCATTGTCGGC |

(SEQ ID NO: 8)

| | | |
|---|---|---|
| m3revB | 25 biotin | CCACAGACAACCCTCATAGTTAGCG |

Proteinase K treated nuclear preparations of FACs sorted COS cells. PCR is carried out using Clontech's Advantage GF polymerase mix and cycled under the following conditions:

1 cycle:

94° C. 1 min.

40 cycles:

94° C. 20 sec.

60° C. 20 sec.

72° C. 20 sec.

Following amplification, duplicate samples were combined and purified using Qiaquick columns (Qiagen Inc., Valencia, Calif.). The PCR products were then digested with Nco1 and Pst1 restriction endonucleases. While SA-magnetic beads or other means for removal of “ends” of fragments increases ligation efficiency, such removal is not required. The digestion product is ligated into a new phage vector and used to transform competent cells by electroporation.

After sub-cloning and electroporation of bacterial cells, the bacteria were plated in top agar and grown overnight at 37° C., plaques were selected from plates and analyzed via PCR using oligonucleotides as follows:

| | | | |
|---|---|---|---|
| MANPglllf | 20 | TTTTGGAGATTTTCAACGTG | (SEQ ID NO: 9) |
| MANPglllr | 20 | TGCTAAACAACTTTCAACAG | (SEQ ID NO: 10) |

However, the oligonucleotides listed previously above are equally useful.

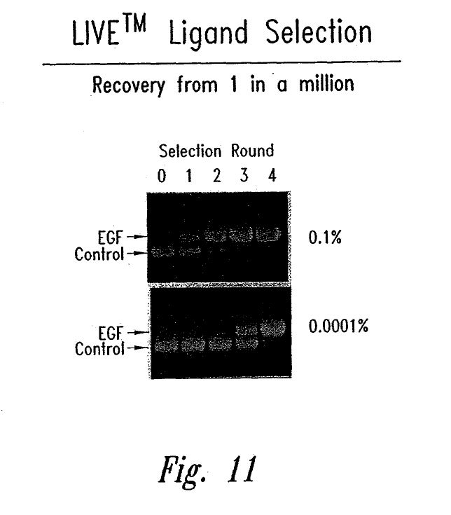

As demonstrated in FIG. 11, enrichment of targeted EGF-phage from 0.1% to 100% EGF-phage was complete after 3 rounds of selection and enrichment from 0.0001% to 100% EGF-phage was complete after 4 rounds of selection. Accordingly, this experiment demonstrates the ability to select a specific ligand expressing phage from a population at dilutions of 1:10³ and 1:10⁶. In addition, while further diluted ligand expressing phage can be detected, further rounds of selection may be necessary.

### Example 20

### Creation of a Sub-library of Peptides that are Internalized and Trafficked to the Nucleus

Phage that display a candidate ligand as a pIII or pVIII fusion on the phage coat are prepared using standard protocols, as discussed above. In the present experiment a phage library (MANP-TN10, Dyax, Corp.) is used. COS cells are plated on 2×10 cm plates at about 105,000 cells/plate. At 24 hours after seeding the cells, phage are added at ˜10¹⁰ pfu/ml. The plates are incubated at 37° C. for an additional 72 hours. The cells are then harvested in Trypsin-EDTA and pelleted. The cells are re-suspended in 0.5 ml of PBS and under-layed with 0.5 mls of nuclear isolation buffer (NIB) and spun at 200×g for 8 minutes at 4° C. (NIB=40% Sucrose, 0.1% DMSO, 2% NP40, 1.6% Triton X-100, 0.2 mM AEBSF (4-(2-Aminoethyl)benzenesulfonyl Fluoride, in PBS). The pellet is then re-suspended in 400 μl 1% NP40 and under-layed with 40% sucrose and again spun at 200×g for 8 minutes at 4° C.

The pellet is then re-suspended in 100 μl PKB (Proteinase K Buffer—50 mM Tris-HCl pH 8.5, 1 mM EDTA, 0.5% Tween-20). 1.4 μl of PK (Proteinase K from Boehringer Mannheim, 14 mg/ml) is added and incubated at 55° C. for 3 hours, then heated to 95° C. for 15 min. The DNA is then pelleted at maximum speed in a microfuge and washed 1 time with 70% ethanol followed by air drying in a hood. The resulting pellet is resuspended in a 20 μl of 10 mM Tris pH 7.2 and the ligand gene III fusion is amplified as described in Example 19, above, except that the cycling program was adjusted as follows:

1 cycle:

94° C. 1 min.

25 cycles:

94° C. 20 sec.

60° C. 20 sec.

72° C. 20 sec.

Following amplification, the product was purified using a Qiaquick column (Qiagen) followed by digestion using NcoI and PstI restriction enzymes, as described above. Biotinylated fragments are removed using streptavidin conjugated beads (Promega Corp., Madison, Wis.), and the resulting sample is ethanol precipitated. The precipitated DNA is then washed and re-suspended in 10 μl of 10 mM Tris pH 8.5 and ligated into the reporter gene carrying phage vector. Following ligation the DNA is ethanol precipitated, washed, and used to transform competent cells (Stratagene electrocompetent cells XI1 Blue MRF′). Phage selected for in this manner (TN10 nuclear selection) are then compared to non-selected phage (TN10 library). The comparison of the pre-selected pool demonstrates that a significant population of the library which did not internalize has been removed in one round of screening, see Table I below:

| Phage | | Dilution | | | Titer | Vol of | Phage | Fraction |
|---|---|---|---|---|---|---|---|---|
| Prep | In/Out | factor | μl plated | plaques | (pfu/ml) | prep (μl) | recovered | of input |
| TN10 | In | 8 | 100 | 123 | 1.23E + 08 | 5000 | 6.15E + 11 | |
| library | | | | | | | | |
| TN10 | Out | 0 | 10 | 127 | 1.27E + 01 | 400 | 5.08E + 03 | 8.26E − 09 |
| nuclear | | | | | | | | |

### Example 21

### Ligand Selection Via Nucleic Acid Binding Domains

A phage library comprising a number of ligand displaying phage is created using a lac operon containing phagemid. The phagemid can be either a reporter gene containing phage (e.g., MEGFP3) or a non-reporter gene containing phagemid (e.g., any vector containing a phage origin of replication, such as pCOMB3, pBS+, pCR, and the like). The MEGFP3 vector has been modified with a mammalian expression cassette designed to express the reporter gene GFP to monitor mammalian cell transduction by the phage. Other vectors include pCANTAB 5 E (Pharmacia Biotech; Piscataway, N.J.) or M13 type 3 or 33 for gene III fusions (see Kay et al., Phage Display of Peptides and Proteins: A Laboratory Manual,, Academic Press, 1996; McConnell etal., Mol. Divers. 1:165-176, 1996). Similarly, the ligand library is cloned into M13 type 8 or 88 vector for fusion to the gene VIII protein (Roberts et al., Methods Enzymol. 267:68-82, 1996; Markland et al., Gene 109:13-19, 1991).

Candidate cell targets are seeded into 6-well culture plates at about 40,000 cells/well. At 24 hours after seeding the cells, phage are added at ˜10¹⁰ pfu/ml. The plates are incubated at 37° C. for an additional 72 hours. Following incubation with the phage library, the target cells are removed from the plate and sorted for GFP expressing cells by FACS or directly lysed and the nucleic acid purified by passing over a sepharose 4B DNA affinity-column having conjugated thereto the lac repressor protein. Prior to affinity purification the cells are lysed and a nuclear extract is produced by following standard procedures, such as those described by Cull et al., Proc. Nat'l. Acad. Sci. USA 89:1865-1869, 1992; Schatz et al., Methods in Enzymology 267:171-191, 1996). The nuclear extract is then passed over the affinity column.

After applying to the column, the column is washed extensively with loading buffer (20 mM Tris-HCl pH 7.2) and eluted with a salt gradient. The resulting DNA containing fractions are pooled, amplified by PCR using the flanking gene III or gene VIII fusion sequences as primer templates, and subcloned back into a phagemid vector for further rounds of enrichment or alternatively for direct sequence characterization.

### Example 22

### Internalized Ligand Sequence Amplification by SV40 Shuttle Vector Transduction

The phagemid-shuttle vector includes the SV40 origin and packaging sequences, as well as SV40 capsid-encoding late genes under control of a promoter functional in target cells. This vector is created by PCR amplification of relevant sequences or direct restriction enzyme digestion and sub-cloning. These sequences can be obtained from commercially available vectors or wild-type virus. Similarly, the phage coat protein fusion, the phage origin and packaging sequences, and bacterial selection markers can be assembled from current phage vectors.

Phage particles expressing a library of ligands as genetic fusions on the coat proteins (gIII or gVIII) are generated by rescuing phagemid containing bacteria with a helper phage, such that the phagemid genome is packaged into the phage particle expressing the ligand which is encoded by that genome. A target cell line containing the SV40 T antigens (either transfected or provided in trans with the VP22 fusion protein as a delivery vehicle) is incubated with the phage particles. Those particles expressing the appropriate ligands deliver the phagemid DNA to the nucleus. Due to the presence of the large T antigen in the cells and the SV40 origin, the DNA replicates. The dsDNA is then packaged into SV40 viral particles due to the presence of the capsid proteins encoded by the SV40 genes also carried on the phagemid genome. The SV40 particles carrying the phagemid then infect other neighboring cells and thus amplifying the internalized ligands until the whole population of cells is infected. Eventually, all permissive cells are lysed due to viral production. Viral particles are harvested from the supernatants and the DNA they contain is analyzed by sequencing to determine the sequence of the ligand responsible for the initial internalization.

### Example 23

### Enhancement of Target Cell Transduction using Heat Shock and/or Genotoxic Treatment

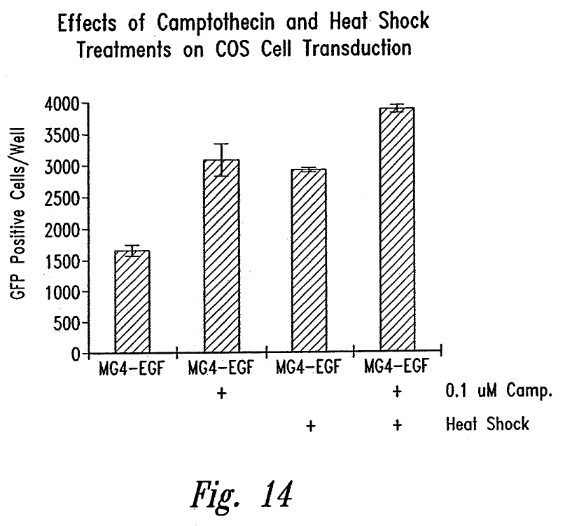

The effects of heat shock and another genotoxic treatment, camptothecin, were tested. Camptothecin is a type I topoisomerase inhibiter that produces DNA strand breaks. Camptothecin is found to enhance transduction efficiency up to 2 fold at 0.1 μM but was inhibitory at higher concentrations (data not shown). EGF displaying phage (MG4-EGF vector) were incubated with COS cells for 24 hours at which time the cells were either not further treated, subjected to a four hour heat shock (42.5° C.), or treated with 0.1 μM camptothecin for four hours. GFP autofluorescent cells were counted at 72 hours after addition of the phage. As depicted in FIG. 14, the results indicate about a 2 fold enhancement of targeted phage mediated transduction with either heat shock or camptothecin. The combination of both treatments resulted in a modest synergistic increase in efficiency over either treatment alone.

### Example 24

### Enhancement of Transduction Utilizing Endosomal Escape Peptide Display

The addition of endosomal escape peptides that are derived from minimal peptide sequences needed for viral endosome escape have been shown to enhance non-viral delivery of condensed DNA (Sosnowski et al., J. Biol. Chem. 271:33647-33653, 1996). Accordingly, such endosomal sequences were tested with the ligand display system described herein. Endosomal escape peptide fusions to pill that are in-frame with both the EGF gene and the pill gene were constructed. The sequence MAEGLFEAIEGFIENGWEGMIDGWYG (SEQ ID NO: 15) (adapted from the INF7 N-terminal sequence of the influenza virus X-31 hemagglutinin subunit HA-2) was added to the N-terminus of EGF in MG4-EGF or PIII in MG4. This sequence is identical to the endosome disruptive peptide (INF7) described by Plank et al. (J. Biol. Chem. 269:12918-12924, 1994) except for the addition of the MAE tripeptide at the N-terminus. It is encoded on 2 overlapping oligonucleotides that are annealed and ligated into the Nco1 site in the MG4 and MG4-EGF vectors.

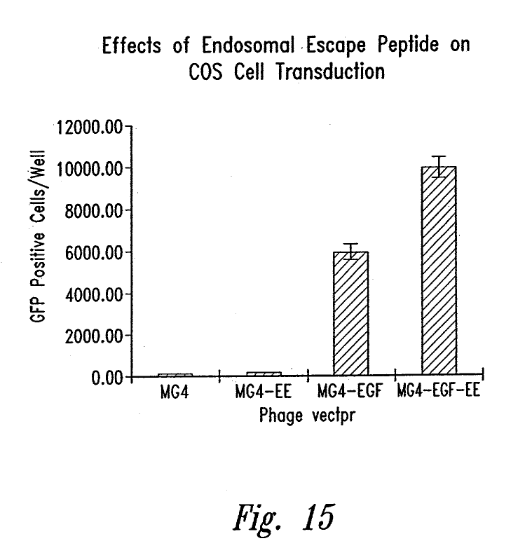

The INF7 containing phage were tested on COS cells to determine transduction efficiency. The results (FIG. 15) show that transduction efficiency is increased about 1.8 fold with the addition of the INF7 sequence relative to the unmodified MG4-EGF phage. The addition of the INF7 sequence in the control phage has no effect on the background levels of transduction observed when transfecting COS1 cells with this phage at a titer of 10¹¹ pfu/ml. Thus, an endosomal disruptive peptide enhances the ability of targeted phage to transduce COS1 cells when presented on the N-terminus of the EGF-pIII coat fusion protein.

### Example 25

### Enhancement of Transduction Utilizing Endosomal Escape Peptide Display and Heat Shock

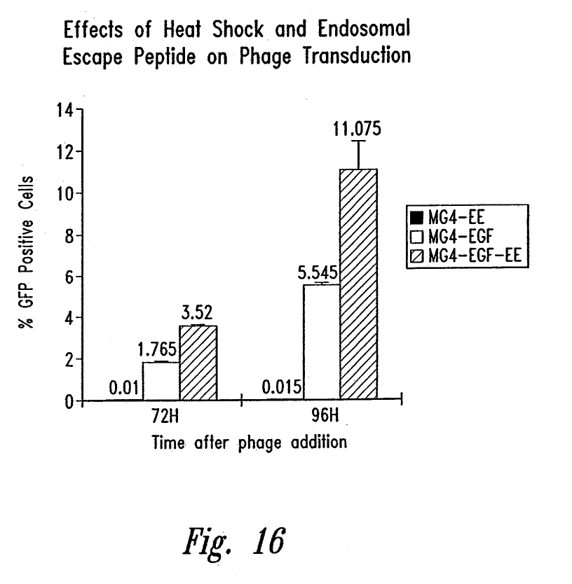

The effect of both heat shock and addition of an endosomal escape peptide was tested in combination to determine whether they effect distinct aspects of the transduction pathway and to test the limits of phage mediated transduction. EGF-phage (MG4-EGF), EGF-phage co-displaying an endosomal escape peptide (INF7 as above) (MG4-EGF-EE) or control phage (MG4) were added to COS1 cells at a titer of about 10¹¹ pfu/ml and incubated for 72 or 96 hours. The cells were subjected to a seven hour heat shock (42.5° C.) at 40 hours after phage addition. The percentage of GFP positive cells was determined by FACS analysis. The results (FIG. 16) show that the effects of heat shock (HS) and the addition of the endosomal escape (EE) sequence are additive. At 72 hours after phage addition, the combination of HS and the EE sequence results in transduction efficiency of 3.5% and at 96 hours in about 11% transduction efficiency. At both time points the addition of the EE peptide results in about 2× the efficiency compared to heat shock alone. The highest transduction efficiency we observe in the absence of HS or the EE peptide is about 2% at 96 hours.

### Example 26

### Construction of Dual Display Vector

The PIII/pVIII dual display vector is constructed by the insertion of a pVIII encoding open reading frame down stream from the stop codon of PIII in a PIII-fusion phagemid vector (e.g., pUC-MG4 (FIG. 17)). This is accomplished by amplifying the mature gVIII gene from MG4 phage using primers that contain EcoR1 restriction endonuclease extensions on their ends.

The DNA sequence encoding the Eco R1 insert for making dual display vector encodes the pEL B Signal Peptide and gVIII gene with restriction enzyme sites Sst II, Xho1 and BamH1 for insertion of peptide fusion s to gene VIII.

| | |
|---|---|
| DNA | (SEQ ID NO: 16) |

GAATTCATGAAATACCTATTGCCTACGGCCGCAGCAGGTCTCCTCCTCTT
AGCAGCACAACCAGCAATGGCCGCGGAGTGACTCGAGGATCCCGCAAAAG
CGGCCTTTAACTCCCTGCAAGCCTCAGCGACCGAATATATCGGTTATGCG
TGGGCGATGGTTGTTGTCATTGTCGGCGCAACTATCGGTATCAAGCTGTT
TAAGAAATTCACCTCGAAAGCAAGCTGATAAGAATTC

| | |
|---|---|
| Protein translation: | (SEQ ID NO: 17) |

MKYLLPTAAAGLLLLAAQPAMAAE.LEDPAKAAFNSLQASATEYIGYAWA
MVVVIVGATIGIKLFKKFTSKAS

“.”=STOP CODON

pEL B leader is in bold type

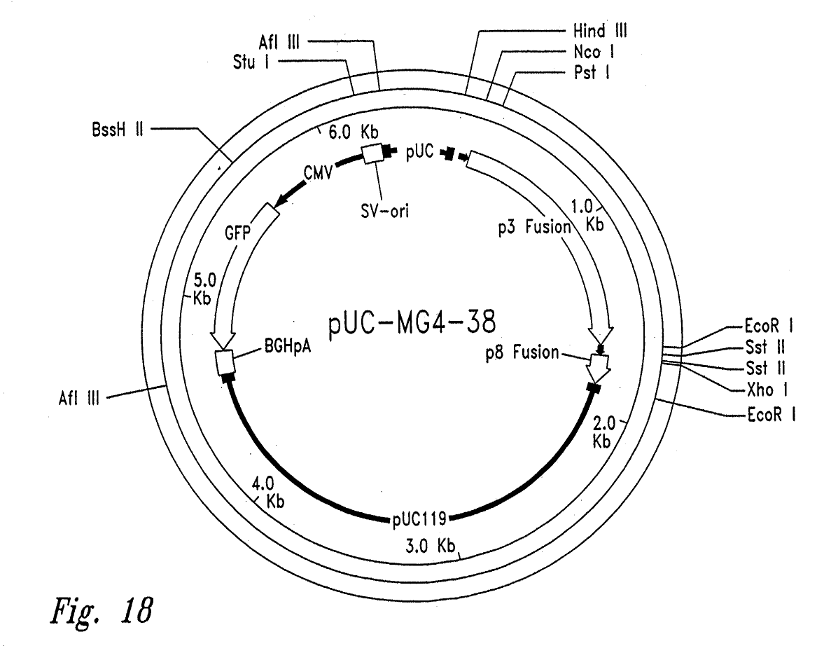

The forward primer contains an additional extension that encodes the pelB secretion signal peptide (Power et al., Gene 113:95-99, 1992) and restriction sites (SstII, XhoI, BamH1) for insertion of peptide or protein encoding fusions in-frame with the pVIII gene. There is an in-frame stop codon at the 5′ end of the pVIII gene such that no pVIII protein is translated unless a peptide or protein encoding sequence is inserted in-frame near the sequences encoding the N-terminus of pVIII. The resulting PCR product is digested with EcoR1 and inserted into the EcoR1 site of the phagemid vector forming a bicistronic message encoding pIII followed by pVIII, both of which are regulated by the lac promoter upstream from the 2 consecutive open reading frames. (See FIG. 18)

### Example 27

### Ligand Selection by using Hirt Supernatant Method

Hirt supernatant method is useful for directly recovering small circular DNAs from transfected or infected cells.

(1) Comparison of Transformation Efficiency Between ssDNA and dsDNA

Varying amounts of purified double-stranded phage DNA and gel isolated single-stranded phage DNA were transformed into XL1-Blue E. coli using electroporation and the number of resulting colonies were counted. The results summarized in Table II demonstrate that dsDNA has a much higher transformation ability compared with ssDNA.

TABLE II
Comparison of Transformation Efficiency Between ssDNA
and dsDNA

| Amount of DNA | Colonies:ssDNA | Colonies:dsDNA |
|---|---|---|
| 10 ng | 50 | 6500000 |
| 1 ng | 10 | 800000 |
| 100 pg | 0 | 350000 |
| 10 pg | 0 | 280000 |

(2) Isolation of Replication Competent dsDNA Using Hirt Supernatant Method

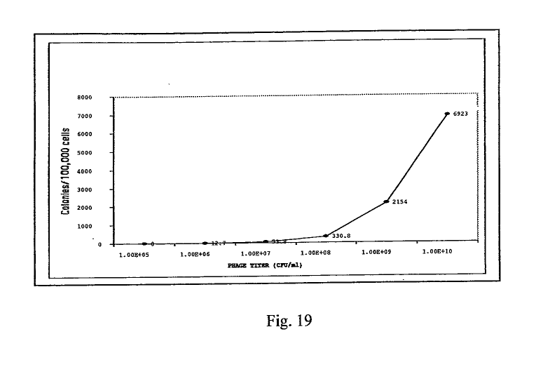

Replication-competent dsDNAs were isolated from phage-infected PC3 cells using the Hirt supernatant method. EGF-targeted phage (1×10⁵-1×10¹⁰ phage) were incubated on PC3 cells for 96 hours, followed by Hirt extraction and electroporation of the DNA into XL1-Blue E. coli. The results summarized in FIG. 19 demonstrated a dose-dependent increase in colonies at phage concentraions of 1×10⁵-1×10¹⁰ (FIG. 1). DNA isolated from cells which had been incubated with 1×10¹⁰ non-targeted control phage resulted in no colonies (data not shown).

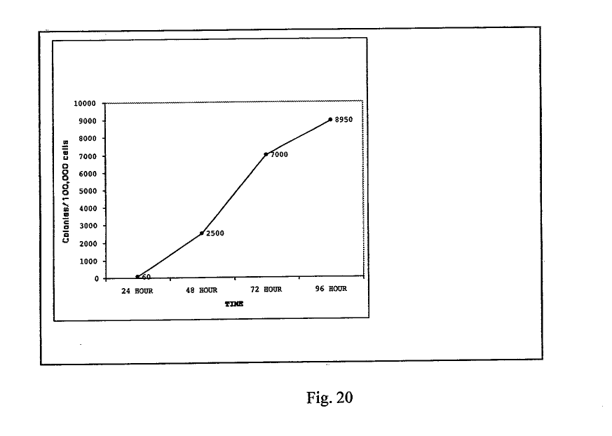

The relationship between the transformation potential of Hirt-extracted DNA from EGF-phage infected PC3 cells to the incubation time after transfection or infection was examined. All experiments were performed the same as described above, except that the cells were incubated with the phage for different time periods before the cells were subject to the Hirt extraction. The results summarized in FIG. 20 demonstrated that the cells incubated with 1×10¹⁰ EGF-targeted phage resulted in a time-dependent increase in the number of colonies.

An experiment was further conducted to test the ability to isolate replication competent phage DNA from cells incubated with EGF-targeted phage diluted up to a million-fold with untargeted phage. EGF-targeted phage (1×10⁴-1×10⁷ cfu/ml) was mixed with untargeted pEGFPN1 phage (1×10¹¹ cfu/ml) and incubated with PC3 cells for 96 hours. DNA was extracted using the Hirt procedure, and DNA was electroporated into XL1-Blue Ecoli. Since the EGF-targeted phage contain an antibiotic resistance marker to ampicillin while pEGFPN1 phage are kanamycin resistant, the relative contribution of transformable DNA from these sources was determined by plating equivelant amounts of tranformation mix onto LB/AMP and LB/KAN plates. The results are summarized in Table III below.

TABLE III
Sensitivity of Hirt Extraction

| Concentration | Ratio EGF phage:Un- | | Colonies |
|---|---|---|---|
| EGF Phage | targeted Phage | Colonies(KAN) | (AMP) |
| 1.0E + 4 cfu/ml | 1:1000000 | 25 | 60 |
| 1.0E + 5 cfu/ml | 1:100000 | 25 | 100 |
| 1.0E + 6 cfu/ml | 1:10000 | 40 | 1600 |
| 1.0E + 7 cfu/ml | 1:1000 | 26 | 2000 |

The sensitivity of the Hirt extractions in combination with cellular fractionation was tested by infecting PC3 cells with a mixture of EGF targeted and untargeted phage at more dilute ratios (1:10000 and 1:100000 and 1:10⁶[twice the number of cells was used for this dilution]) and analyzing Hirt DNA from cytoplasmic and nuclear fractions. The number of colonies increased with increasing amount of EGF in the initial samples. The percentage of colonies identified as EGF by sequencing also increased with increasing amounts of EGF in the original phage samples. The data suggest that the level of detection in one round of selection using Hirt extraction screening is at least 1:10⁶

TABLE IV
Sensitivity of Hirt Extraction With Cellular Fractionation

| | Ratio | | | | |
|---|---|---|---|---|---|
| | EGF phage: | | | Colo- | |
| Concentration | Untargeted | Colo- | | nies: | |
| EGF Phage | Phage | nies:Cyt | % EGF | Nuc | % EGF |
| 1.0E + 4 cfu/ml | 1:1000000 | 130 | 11.10% | 8 | 12.50% |
| 1.0E + 5 cfu/ml | 1:100000 | 171 | 26.70% | 10 | 40.00% |
| 1.0E + 6 cfu/ml | 1:10000 | 471 | 64.30% | 63 | 71.10% |

In order to validate the Hirt procedure as an alternative to conventional screenings, Hirt isolation was used to select a fragmented EGF gene library for functional EGF fragments. Library phage (1×10¹⁰ cfu/ml) were added to one dish containing 2×10⁵ PC3 cells. 96 hours later cells were washed, isolated, and subjected to Hirt DNA isolation. Hirt DNA was then used to transform bacteria. A total of 110 colonies were obtained. Random clones were sequenced. A total of 3/19 clones that were sequenced contained the mature EGF sequence. The remaining sequences localized to other regions of the EGF precursor gene.

### Example 28

### Construction of pBADamp Vector and cDNA Library

The pBADamp vector was constructed to select for in frame libraries. The vector pBAD/pIII (Invitrogen Corp., Carlsbad, Calif.) was used as a template for inverse PCR. Two primers were used:

ampinv (GGCTCGAGCGGCCGCTGCAGCTCACCCAGAAACGCTGGTGAAAG, SEQ ID NO: 19)

and

pbadinv (GGCTCGAGGGCCGGCCGGCGCGCCCGCCATGGTGCTATGGCTAT-AGMCG, SEQ ID NO: 20),

eliminating the MCS and sequences upstream of the ampicillin resistance gene ORF. The PCR conditions were as follows: 94° C. for 5 minutes, followed by 30 cycles of 94° C. for 30 seconds, 60° C. for 30 seconds, 72° C. for 3 minutes. A long extension at 72° C. for 10 minutes was performed. The linear PCR product (3.4 kb) was digested with XhoI and self-ligated to create pBADamp. The pBADamp vector contains a new MCS with the cloning sites (NcoI, AscI, FseI, XhoI, NotI, and PstI), including three restriction enzymes recognizing eight nucleic acid residues for fragmented cDNA libraries. This vector fuses the PIII signal sequence to the ampicillin resistance gene so that only in frame inserts will allow expression of β-lactamase. This vector was verified by sequence analysis.

A fragmented cDNA library was constructed in pBADamp and in frame clones were selected. Fragmented cDNAs from human placenta were cloned into NcoI and NotI digested pBADamp that was also treated with calf intestine phosphotase. Colonies were selected on media containing 60 μg/ml ampicillin and 0.2% arabinose to induce the pBAD promoter. 500,000 cDNA clones were made into a large DNA prep and clones were assayed for in frame sequences. Sequence analysis indicated 96.7% cDNA inserts (29/30) were in frame with pIII.

### Example 29

### Selection of In-frame cDNA Inserts

The pBADamp vector was constructed as disclosed above. EGF ligand and 3 out of frame clones from the EGF library were inserted into this vector and plated on media containing either glucose (2%) (promoter off) or arabinose (0.2%) (promoter on). None of the out of frame sequences yielded colonies on either glucose or arabinose media. EGF clones grew on the arabinose media (500-1000 colonies), and only 3 colonies grew on the glucose media. This experiment summarized in Table V indicates that there is a strong preference in the vector for in-frame inserts.

TABLE V
Selection of In-Frame cDNA Inserts

| | 2X YT 2% glucose | 0.2% arabinose |
|---|---|---|
| Vector control | 0 | 1 colony |
| Rd 0 #5 unselected | 1 colony | 3 colonies |
| Rd 1 #2 selected | 0 | 0 |
| Rd 1 #10 selected | 0 | 3 colonies |
| EGF ligand | 3 colonies | ˜1000 colonies |

### Example 30

### Construction of pUC-based Phagemid Display Vectors

pUC-based vectors deleting the N-terminal 188, 198, 207 or 250 amino acids of PIII were constructed. It has been established that deletion of the N-terminal 250 amino acids of pIII has led to increase in stability of the EGF-pIII fusion and resulted an increase in cell transduction. Phage carrying the different peptides were made using these vectors with deletions of 188, 198, or 207 amino acids, and compared with the construct with a deletion of 250 amino acids. When EGF-pIII fusions were constructed in the 188, 198, and 207 deletion vectors and resulting phage compared to 250-EGF phage, all phage had similar levels of monophage and PIII display compared to 250-EGF except 188-EGF which had reduced levels of monophage. All phage resulted in similar levels of transduction in PC cells as pUC250-EGF. When a random peptide library (CX8C) was constructed in all four vectors and analyzed, two of the alternative deletions (198 and 207) have improved monophage and PIII display compared to the 250 deletion vector. The 188 deletion vector showed only modest increase in PIII display. When a cDNA library derived from human placental RNA was constructed in all four deletion vectors and phage analyzed by multiphage analysis and western blotting, the 198 truncation results in the best display. When several other ligands (neurotensin, MSH, and FGF-Heparin peptide) were constructed in the deletion vectors and phage were analyzed, the three shorter deletion vectors (188, 198, 207) showed improved display characteristics compared to the 250 deletion mutant.

### Example 31

### Subcloning cDNA Inserts from pBAD-amp Plasmid to pUC-based Phagemid Display Vectors

Placental cDNAs were digested from the pBADamp vector with NcoI and NotI, and ligated into pUC198, pUC207 and pUC250. These were analyzed by sequence, Western blot and multiphage. Analysis of the cDNA sequences from these libraries and of the previous pUC250 cDNA library showed that the original cDNA library in pUC250 (not selected for in frame inserts) has 10.5% in-frame inserts (2/19) from test ligations/pre-rescue or 19.2% in frame (5/26) by PCR of phage particles, that pBADamp library of cDNA's (selected in-frame inserts with ampicillin resistance) contains 96.7% in frame (29/30) clones from library, and that new libraries made from cDNA inserts from pBADamp (in-frame inserts selected by ampicilin resistance) contain 71.4% (10/14) in-frame inserts of pUC198 library from test ligations, 77% (10/13) in-frame inserts of pUC207 library from test ligations, and 87% (13/15) in-frame inserts of pUC250 library from test ligations.

### Example 32

### Selection of Intracellular Trafficking Peptides

In order to improve the efficiency of phage-mediated transduction, it has been attempted to display sequences on the phage that will lead to greater endosomal escape, nuclear localization and/or DNA replication and transcription. A random peptide library (CX₈C)GGGS (SEQ ID NO: 21) as a N-terminal extension of EGF-pIII was constructed and screened on PC3 cells. 24 hours after transfection, low molecular weight DNA was extracted by Hirt supernatant method from cytoplasmic and nuclear fractions. The Hirt extractions were used to transform E. coli. Phage libraries were produced from both the cytoplasmic and nuclear colonies and used as input phage for a second round of screening.

The ratio of colonies obtained from the cytoplasm versus the nuclear (N/C ratio) suggested that phage were enriched for sequences that increased phage targeting to the nucleus. Sequence analysis from random clones revealed a collapse in sequence diversity. One sequence from round 2 (CQQESGKQSC) (SEQ ID NO: 22) continued to be enriched in subsequent rounds and represented 40% of the isolated phage by round 4. In addition, the N/C ratio of this individual phage is approximately 20-fold higher than EGF-phage or the phage population of the unselected library. Two other sequences were similarly identified.

### Example 33

### selection of Ligand Targeted Phage by Drug Resistance

An EGF targeted phagemid vector was created by substitution of the GFP gene in pUC-250 with a neomycin phosphotransferase gene (neo). PC-3 cells were transfected with various doses of pUC-250 neo. At 96 hours after transfection, cells were transferred to selective medium containing G418. Colonies of drug resistant cells were stained with Geimsa and counted after 10 days. It has been demonstrated a dose dependent increase in colonies with increasing dose of pUC-250 neo.

### Example 34

### Comparative Amplification and Quantification of Circular Genetic Packages

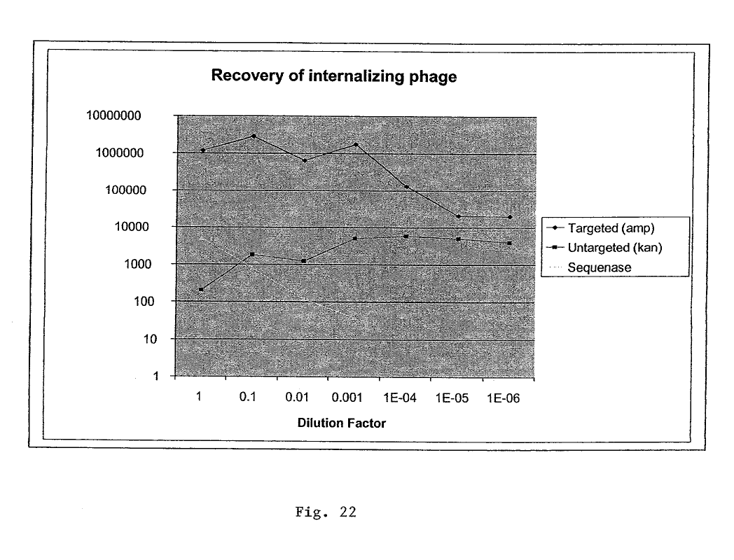

Nucleic acid sequences are recovered from cells transfected with a genetic package display vector by DNA amplification methods, such as the rolling circle method using phi 29 polymerase or single-strand conversion using Sequenase™, for example. Equal amounts of cell lysates containing template circular genetic packages (i.e., from internalized phage vectors) were mixed with Phi 29 polymerase or Sequenase™, as described in Example 35. Amplification products are digested with Pst1, religated and transformed into competent bacterial host cells. Transformed host cells were plated and incubated overnight at 37° C., and the number of colonies counted. Following transformation of bacterial host cells, the number of antibiotic resistant colonies counted correlates with the ratio of targeted to non-targeted phage internalized by PC-3 mammalian cells. A significantly higher number of antibiotic resistant colonies from amplification reactions using Phi 29 polymerase were counted, compared to parallel amplification reactions that were performed with Sequenase™ (FIG. 22). Biopanning showed similar results.

### Example 35

### A Method for Sensitive Amplificiation of Circular Genetic Packages using Phi 29 Polymerase

We describe a sensitive and selective method for the detection and identification of internalizing ligands using Phi 29 DNA polymerase amplification of a ligand targeted vector comprising a circular genetic package. The disclosed amplification method provides a significantly higher degree of sensitivity compared to conventional identification procedures. The method set forth is selective and can be applied to any biological sample containing an internalized circular genetic package. When, for example, the circular genetic package is a targeted phage display expression vector, this procedure recovers the internalized circular genetic package, thereby identifying the encoded ligand mediating internalization. The biological sample may, but need not be a tissue or a cell from a patient with a disease such as cancer, or a subject patient that is otherwise disease free (normal). According to the methods of this Example, the biological source may be derived from any animal or any plant known to or suspected of containing an internalized circular genetic package.

Another advantage of this method is to overcome the problem of loss of infectivity known to those of ordinary skill in the art. For example, it is common to attempt to isolate infective phage particles representing internalized (targeted) genetic packages from cell lysates. However, internalized phage particles that are unavoidably damaged during internalization or preparation of the lysate cannot infect a bacterial host cell. The targeting ligand mediating internalization, therefore, cannot be identified (a false negative). The method described here overcomes this problem by specifically producing (amplifying) a large number of internalized genetic packages, without requiring the recovered phage particle to be infective; the amplified DNA is then incorporated into a host cell, which may be detected using a selectable marker such as resistance to the antibiotic ampicillin. According to the method described here, small circular DNA can, for example, be recovered from cells or cell lysates according to the procedure described by Hirt (1967, J. Mol. Biol. 26(2):365-9). The single stranded phage DNA so isolated is then converted into double-strand DNA by addition of a reaction mixture comprising one or more selected oligonucleotide primers or a plurality of random oligonucleotide primers, nucleotide triphosphates and DNA polymerase (e.g., sequenase or Phi 29 polymerase); the reaction products are then used to transform bacteria.

When using Sequenase™, according to procedures currently used in the art, the recovery of a targeted phage genetic package is approximately 1 out of 100,000 input phage particles. However, as set forth here, recovery is greatly improved when amplification is performed using Phi 29 polymerase, according to the instant application, where internalized phage genetic packages are enriched over 1 million fold in one round of selection (see FIG. 22). In addition, the procedure described here eliminates or greatly reduces (i) the formation of artifacts associated with the commonly used PCR amplification of DNA, and (ii) the necessity of subcloning amplified inserts. In brief, any extract (lysate) derived from a tissue and/or cell can be evaluated for the presence of an internalized circular genetic package. A cell and/or tissue sample, lysate and/or extract, is prepared and contacted with Phi 29 polymerase, or mechanistically equivalent thereof, as would be recognized by those of ordinary skill, under conditions and for a time sufficient to amplify a detectable amplification product, according to procedures available to the skilled artisan.

In this particular Example, mammalian cells are incubated with two bacteriophage-based vectors, each containing a circular genetic package, but different selectable and identifiable reporter genes. Accordingly, one such phage vector, containing a circular genetic package, also includes an encoded mammalian cell-targeting component (ligand) displayed on the phage surface, a targeted phage display vector, plus the selectable marker conferring ampicillin resistance. The other phage vector does not contain a displayed ligand (i.e., is non-targeted), although it does encode a different selectable marker conferring kanamycin resistance. In order to compare the detection efficiency of the subject invention using Phi 29 polymerase with another DNA detection procedure using Sequenase™, cell lysates containing the targeted and subsequently internalized genetic package are prepared, divided into two portions, one is subjected to Sequenase™ and the other subjected to Phi 29-mediated amplification, a general rolling circle amplification mechanism (Genome Res. 11(6):1095-9).

Mammalian cells plated on cell culture dishes are contacted with a mixture of targeted phage display vector and non-targeted (control) phage vector. The mixture of phage vectors applied to cells may be adjusted so that cells are contacted with phage mixtures containing different ratios of targeted to non-targeted phage vectors. As shown in FIG. 22, a targeted phage particle carrying an ampicillin resistance gene was mixed with a non-targeted phage particle carrying a kanamycin resistance gene at ratios ranging from an equal amount of both phage vectors, to, 1 targeted phage particle in the presence of one million non-targeted phage particles, a dilution of 1 in 1,000,000. The phage were applied to PC-3 mammalian cells and incubated for 2 hours at 37° C. Cells were then washed in low pH buffer to remove externally bound but not internalized phage particles. Cells are removed from the culture dish with trypsin/EDTA, washed three times with PBS, pelleted and cell lysates containing circular genetic packages (templates) are prepared for amplification according to procedures described by Hirt (1967, J. Mol. Biol. 26(2):365-9). The Hirt extracted DNA preparation containing the phage circular genetic packages is was then incubated with a mixture of random hexamer oligonucleotide primers, dNTPs and Phi 29 DNA polymerase at 30° C., for 2-16 hours. The Phi 29 polymerase amplification reaction is performed following procedure recommended by the manufacturer (Amersham Inc.). Other polymerases may be used to amplify the circular genetic package by this rolling circle procedure, such as exo(−)BST DNA polymerase or a polymerase from an appropriate circular DNA virus. The amplification product was then digested for 4-16 hours with a restriction endonuclease (e.g., Pst1) that cuts the phage DNA at one location. Digested DNA was purified and ligated for 2-4 hours at 16° C. using DNA ligase, and then transformed into suitable bacterial host cells by, for example, electroporation, according to conditions recommended by the manufacturer (BioRad Inc.). Transformed bacteria are plated on agar-based media containing a selection reagent (antibiotic) such as ampicillin or tetracycline. Colonies are recovered, sequenced to identify sequences encoding the internalizing ligand. Colonies are also pooled and used to prepare phage for subsequent rounds of screening.

### Example 36

### Construction of the pRV-198-EGF Phagemid

The phagemid pRV-198-EGF is designed to produce an RNA message that encodes the drug resistance protein neomycin phosphotransferase following the appropriate events (FIG. 23). Neomycin phosphotransferase (neoR or neo/kanR) confers resistance to the drug G418 in mammalian cells and to kanamycin in bacterial cells. The neo/kanR gene is interrupted in the pRV-198 retro-vector by intervening, non-conding or intron sequences. The neo/kanR gene lies downstream of the CMV promoter (active in mammalian cells) and the EM7 promoter (active in bacterial cells). The EGF ligand insert lies downstream of the neo/kanR gene and upstream of pIII coat protein sequence. The vector sequences are arranged so that the RNA message produced from the pRV-198 vector includes the neo/kanR gene, continues through the EGF-pIII fusion encoding sequences, and continues through the pUC origin of replication, which allows high copy plasmid replication in E. coli.

### Example 37

### Detection of Internalizing/transducing Phage by RNA Amplification

To identify phage displayed ligands that promote internalization of phage leading to nuclear localization and gene expression, phage are produced from the pRV-198-EGF phagemid (displaying EGF ligand fused to PIII coat protein) and contacted with PC-3 human prostate carcinoma cells. After 72-96 hours, the cells are washed to remove bound phage and lysed according to standard procedures. Total RNA or polyA+mRNA is prepared from the lysed cells using standard protocols, and RT-PCR is performed to amplify the phage encoded mRNA using reverse transcriptase and oligonucleotides primers that bind specifically to the message produced to produce a cDNA that includes the bacterial promoter, the neo gene, the EGF-PIII fusion gene and the pUC origin of replication. The primers have extended sequences that create restriction enzyme sites at each end of the cDNA. The ends of the amplified linear cDNA are cut with the appropriate restriction endonuclease and ligated to itself using T4 DNA ligase to form closed circular DNA. The closed circular DNA is used to transform E. coli bacteria, and the neomycin producing transformants are selected by growing on kanamycin containing media.

### Example 38

### Selection of Internalizing Phage using Phi 29 Polymerase

A targeted phage particle carrying an ampicilling resistance gene was mixed with a non-targeted phage particle carrying a kanamycin resistance gene at ratios ranging from equal amounts of each to a one in one million dilution of the targeted phage in an excess of untargeted phage. The phage were contacted with PC-3 cells for two hours, cells were washed in low pH buffer to remove externally bound phage and circular DNA prepared from the cell lysates.

The DNA was amplified using phi 29 polymerase and used to transform host bacteria by electroporation. The ratio of ampicillin resistant colonies to kanamycin resistant colonies obtained indicated the relative amounts of each phage in the selected population. After one round of selection in a mixture containing a million-fold excess of non-targeted phage, there was a four-fold excess of targeted phage, which indicated over a million-fold enrichment (data not shown).

### Example 39

### Enhancement of Target Cell Transduction using DEAE-dextran

The effect of the transfection reagent DEAE-dextran on EGF targeted phage transduction using a GFP transgene was examined. Phage were incubated with cells and DEAE-dextran for six hours in serum-free medium, washed, and returned to serum-containing mediua. GFP positive cells were assesses at 96 hours after phage addition.

At concentrations of DEAE-dextran typically used for DNA transfection (200 ug/ml), EGF-targeted phage transduction increased from 0.5% to about 3.5% GFP positive cells, while non-specific transduction increased from 0% to only about 0.5% (data not shown). Significantly, the mean fluorescence intensity of cells transduced by targeted phage was at least ten-fold higher with DEAE-dextran, indicating that DEAE-dextran caused increased expression of the reporter gene. (data not shown)

DEAE-dextran was titered to determine the minimal dose needed to increase transduction efficiency with minimal toxicity. Phage were preincubated with DEAE-dextran for 30 minutes at 30° C. before adding the phage complex to the cells. The maximal effect of DEAE-dextran was obtained at 0.6 ug/ml with little variation in transduction efficiency at doses up to 25 ug/ml (data not shown). Transduction efficiency decreased by about half at 0.3 ug/ml DEAE-dextran. Furthermore, even at low doses of DEAE-dextran (0.6 ug/ml), there was still a significant increase in gene expression, as indicated by a nearly two-fold increase in mean fluorescence and a ten-fold increase in the percentage of GFP positive cells (data not shown).

The specificity of EGF-targeted phage was examined by competing with an excess of free EGF ligand. Free EGF significantly reduced transduction efficiency by about 72%, indicating EGF-targeted phage transduction was specific for the EGF receptor in the presence of DEAE-dextran (data not shown).

From the foregoing it will be appreciated that, although specific embodiments of the invention have been described herein for purposes of illustration, various modifications may be made without deviating from the spirit and scope of the invention. Accordingly, the invention is not limited except as by the appended.

We claim:

1. A vector for selecting in-frame inserts, comprising a first nucleic acid sequence encoding a genetic package coat protein signal sequence and a second nucleic acid sequence encoding a selectable marker, wherein the in-frame insertion of an insert nucleic acid sequence between the first and second nucleic acid sequences allows expression of the selectable marker.

2. The vector of claim 1, further comprising an inducible promoter.

3. The vector of claim 2, wherein the inducible promoter is araC.

4. The vector of claim 1, wherein the bacteriophage coat protein is selected from the group consisting of M13 gIII, M13 gVI, M13 gVII, M13 gVIII, M13 gIX, fd minor coat protein PIII, lambda D protein, lambda phage tail protein pV, fr coat protein, Ø29 tail protein gp9, MS2 coat protein, T4 smaI outer capsid protein, T4 nonessential capsid scaffold protein IPIII, T4 lengthened fibritin protein gene, PRD-1 gene III, Qβ3 capsid protein, and P22 tailspike protein.

5. The vector of claim 4, wherein the bacteriophage coat protein is M13 gIII.

6. The vector of claim 1, wherein the second nucleic acid sequence is an antibiotic resistance gene.

7. The vector of claim 6, wherein the antibiotic resistance gene is an ampicillin resistance gene.

8. The vector of claim 1 or 6, wherein the vector has one insert.

9. A method for making a library of vectors with in-frame inserts, comprising: introducing a mixture of nucleic acid inserts into the vector of claim 1, resulting in insert-containing vectors; transducing the insert-containing vectors into bacteria; and selecting bacterial clones that grow under selective conditions specific for the selectable marker.

10. The method of claim 9, further comprising recovering the vectors from the bacterial clones.

11. The method of claim 9, further comprising isolating the inserts from the vectors and inserting the isolated inserts into a phage genome or a phagemid.

12. The method of claim 11, wherein the phagemid is a pUC-based phagemid.

13. The method of claim 12, wherein the pUC-based phagemid is selected from the group consisting of pUC198, pUC207, and pUC250.

14. The method of claim 11, wherein the phage genome or phagemid comprise a marker gene capable of being expressed in a mammalian cell.

15. The method of claim 9, wherein the mixture of nucleic acid inserts is derived from a cDNA library.

16. The method of claim 9, wherein the mixture of nucleic acid inserts is derived from an antibody gene library.

17. The method of claim 9, wherein the mixture of nucleic acid inserts is derived from a peptide gene library.

18. The method of claim 9, wherein the mixture of nucleic acid inserts is derived from a mutein library.

19. A library of vectors with in-frame inserts produced by the method of claim 9.

20. A library of vectors with in-frame inserts produced by the method of claim 11.

21. The library of claim 20, wherein the vector is a pUC-based phagemid.

22. The library of claim 21, wherein the pUC-based phagemid is selected from the group consisting of pUC198, pUC207, and pUC250.

23. The library of claim 20, wherein the vectors further comprise a marker gene capable of being expressed in a mammalian cell.

24. The library of claim 19, wherein the inserts are derived from a cDNA library.

25. The library of claim 19, wherein the inserts are derived from an antibody gene library.

26. The library of claim 19, wherein the inserts are derived from a random peptide gene library.

27. The library of claim 19, wherein the inserts are derived from a mutein library.

28. A library of bacteriophage comprising in-frame inserts produced by the method of claim 11.

29. A method of identifying a target cell or tissue for a ligand, comprising:
(a) contacting a ligand displaying genetic package comprising an in-frame insert with a cell(s) or tissue(s), wherein the package comprises a nucleic acid sequence encoding a detectable product; and
(b) detecting product in the cell(s) or tissue(s), and thereby identifying a target cell or tissue for an internalizing ligand.

30. The method of claim 29, wherein the package is contacted with the cell(s) or tissue(s) in vivo.

31. The method of claim 29, wherein the package is contacted with the cell(s) or tissue(s) for 24-96 hours prior to the detection.

32. The method of claim 29, wherein the package is contacted with the (cell)s or tissue(s) for 48-72 hours prior to the detection.

33. The method of claim 29, wherein the package is contacted with the (cell)s or tissue(s) for 72-96 hours prior to the detection.

34. The method of claim 29, wherein the package is contacted with the (cell)s or tissue(s) for at least 96 hours prior to the detection.

35. The method of claim 29, wherein the product is detected in substantially non-vasculature cells.

36. The method of claim 29, wherein the product is detected in parenchymal cells.

37. The method of claim 29, wherein the product detected is a polypeptide.

38. The method of claim 37, wherein the polypeptide confers drug resistance to the cell(s) or tissue(s).

39. The method of claim 29, wherein the product detected is an mRNA.

40. The method of claim 39, wherein the mRNA is detected by RT-PCR.

41. The method of claim 29, wherein the product detected is DNA.

42. The method of claim 41, wherein the DNA is detected by Hirt extraction.

43. The method of claim 41, wherein the DNA is detected by PCR.

44. The method of claim 41, wherein the DNA is detected by rolling circle amplification.

45. The method of claim 44, wherein the rolling circle amplification is performed using Phi 29 polymerase or exo(−) BST DNA polymerase.

46. The method of claim 41, wherein the DNA is detected by its ability to bind a specific DNA binding protein or nucleic acid.

47. The method of claim 29, wherein the nucleic acid sequence encoding the detectable product comprises an intron.

48. The method of claim 47, wherein the detectable product is expressed following mRNA processing in a mammalian cell.

49. The method of claim 29, wherein the method is a high throughput method and the cells are immobilized on an array.

50. The method of claim 29, wherein the cell(s) or tissue(s) are contacted with a library of said ligand displaying genetic packages.

51. A method of selecting an internalizing ligand displayed on a genetic package, comprising:
(a) contacting a library of ligand displaying genetic packages comprising in-frame inserts with a cell(s), wherein each package comprises a nucleic acid sequence encoding a detectable product, and
(b) detecting product in the cell(s); and thereby selecting an internalizing ligand displayed on a genetic package.

52. A method of identifying an internalizing ligand displayed on a genetic package, comprising the method of claim 51 and further comprising recovering a nucleic acid molecule encoding the internalizing ligand from cell(s) expressing the detectable product, and thereby identifying an internalizing ligand.

53. The method of claim 51 or 52, wherein the library is contacted with the cell(s) in vivo.

54. The method of claim 51 or 52, wherein the library is contacted with the cell(s) for 24-96 hours prior to the detection.

55. The method of claim 51 or 52, wherein the library is contacted with the cell(s) for 48-72 hours prior to the detection.

56. The method of claim 51 or 52, wherein the library is contacted with the cell(s) for 72-96 hours prior to the detection.

57. The method of claim 51 or 52, wherein the library is contacted with the cell(s) for at least 96 hours prior to the detection.

58. The method of claim 51 or 52, wherein the product is detected in substantially non-vasculature cells.

59. The method of claim 51 or 52, wherein the product is detected in parenchymal cells.

60. The method of claim 51 or 52, wherein the product detected is a polypeptide.

61. The method of claim 60, wherein the polypeptide confers drug resistance to the cell(s).

62. The method of claim 51 or 52, wherein the product detected is an mRNA.

63. The method of claim 62, wherein the mRNA is detected by RT-PCR.

64. The method of claim 51 or 52, wherein the product detected is DNA.

65. The method of claim 64, wherein the DNA is detected by Hirt extraction.

66. The method of claim 64, wherein the DNA is detected by PCR.

67. The method of claim 64, wherein the DNA is detected by rolling circle amplification.

68. The method of claim 67, wherein the rolling circle amplification is performed using Phi 29 polymerase or exo(−)BST DNA polymerase.

69. The method of claim 64, wherein the DNA is detected by its ability to bind a specific DNA binding protein or nucleic acid.

70. The method of claim 51 or 52, wherein the nucleic acid sequence encoding the detectable product contains an intron.

71. The method of claim 70, wherein the detectable product is expressed following mRNA processing in a mammalian cell.

72. The method of claim 51 or 52, wherein the method is a high throughput method and the cells are immobilized on an array.

73. A method of selecting internalizing ligand/anti-ligand pairs, comprising:
(a) contacting a library of ligand displaying genetic packages comprising in-frame inserts with a cell(s), wherein each package comprises a nucleic acid sequence encoding a detectable product, and wherein the cell(s) expresses an anti-ligand-receptor fusion protein on its surface; and
(b) detecting product expressed by the cell(s), and thereby selecting internalizing ligand/anti-ligand pairs.

74. A method of identifying an internalizing ligand and/or an anti-ligand of a ligand/anti-ligand pair, comprising the method of claim 73 and further comprising recovering a nucleic acid molecule encoding an internalizing ligand and/or a nucleic acid molecule encoding an internalizing anti-ligand from cell(s) that grow under the selective conditions, and thereby identifying a ligand and/or anti-ligand of an internalizing ligand/anti-ligand pair.

75. The method of claim 73 or 74, wherein the library is contacted with the cell(s) in vivo.

76. The method of claim 73 or 74, wherein the library is contacted with the cell(s) for 24-96 hours prior to the detection.

77. The method of claim 73 or 74, wherein the library is contacted with the cell(s) for 48-72 hours prior to the detection.

78. The method of claim 73 or 74, wherein the library is contacted with the cell(s) for 72-96 hours prior to the detection.

79. The method of claim 73 or 74, wherein the library is contacted with the cell(s) for at least 96 hours prior to the detection.

80. The method of claim 73 or 74, wherein the product is detected in substantially non-vasculature cells.

81. The method of claim 73 or 74, wherein the product is detected in parenchymal cells.

82. The method of claim 73 or 74, wherein the product detected is a polypeptide.

83. The method of claim 82, wherein the polypeptide confers drug resistance to the cell(s).

84. The method of claim 73 or 74, wherein the product detected is an mRNA.

85. The method of claim 84, wherein the mRNA is detected by RT-PCR.

86. The method of claim 73 or 74, wherein the product detected is DNA.

87. The method of claim 86, wherein the DNA is detected by Hirt extraction.

88. The method of claim 86, wherein the DNA is detected by PCR.

89. The method of claim 86, wherein the DNA is detected by rolling circle amplification.

90. The method of claim 89, wherein the rolling circle amplification is performed using Phi 29 polymerase or exo(−)BST DNA polymerase.

91. The method of claim 73 or 74, wherein the nucleic acid sequence encoding the detectable product comprises an intron.

92. The method of claim 91, wherein the detectable product is expressed following mRNA processing in a mammalian cell.

93. The method of claim 73 or 74, wherein the method is a high throughput method and the cells are immobilized on an array.

94. A method of identifying a target cell or tissue for an internalizing ligands, comprising:
(a) contacting a ligand displaying genetic package comprising a nucleic acid sequence encoding a detectable mRNA that is expressed upon internalization of the package with a cell(s) or a tissue(s); and
(b) detecting the detectable mRNA expressed by the cell(s) or tissue(s), and thereby identifying a target cell or tissue for internalizing ligands.

95. The method of claim 94, wherein the library is contacted with the cell(s) or tissue(s) in vivo.

96. The method of claim 94, wherein the ligand displaying genetic packages comprises an in-frame insert.

97. The method of claim 94, wherein the ligand displaying genetic package is contacted with the cell(s) or tissue(s) for 24-96 hours prior to the detection.

98. The method of claim 94, wherein the ligand displaying package is contacted with the cell(s) or tissue(s) for 48-72 hours prior to the detection.

99. The method of claim 94, wherein the ligand displaying package is contacted with the cell(s) or tissue(s) for 72-96 hours prior to the detection.

100. The method of claim 94, wherein the ligand displaying package is contacted with the cell(s) or tissue(s) for at least 96 hours prior to the detection.

101. The method of claim 94, wherein the mRNA is detected in substantially non-vasculature cells.

102. The method of claim 94, wherein the mRNA is detected in parenchymal cells.

103. The method of claim 94, wherein the mRNA is detected by RT-PCR.

104. The method of claim 94, wherein the nucleic acid sequence encoding the detectable mRNA comprises an intron.

105. The method of claim 104, wherein the detectable mRNA is expressed following mRNA processing in a mammalian cell.

106. The method of claim 94, wherein the method is a high throughput method and the cells are immobilized on an array.

107. A method of selecting an internalizing ligand displayed on a genetic package, comprising:
(a) contacting a library of ligand displaying genetic packages comprising a nucleic acid sequence encoding a detectable mRNA that is expressed upon internalization of the package with a cell(s), and
(b) detecting the detectable mRNA expressed by the cell(s); and thereby selecting an internalizing ligand displayed on a genetic package.

108. A method of identifying an internalizing ligand displayed on a genetic package, comprising the method of claim 44 and further comprising recovering a nucleic acid molecule encoding the internalizing ligand from cells expressing the detectable mRNA, and thereby identifying an internalizing ligand.

109. The method of claim 107 or 108, wherein the library is contacted with the cell(s) in vivo.

110. The method of claim 107 or 108, wherein the ligand displaying genetic packages comprise in-frame inserts.

111. The method of claim 107 or 108, wherein the library is contacted with the cell(s) for 24-96 hours prior to the detection.

112. The method of claim 107 or 108, wherein the library is contacted with the cell(s) for 48-72 hours prior to the detection.

113. The method of claim 107 or 108, wherein the library is contacted with the cell(s) for 72-96 hours prior to the detection.

114. The method of claim 107 or 108, wherein the library is contacted with the cell(s) for at least 96 hours prior to the detection.

115. The method of claim 107 or 108, wherein the mRNA is detected in substantially non-vasculature cells.

116. The method of claim 107 or 108, wherein the mRNA is detected in parenchymal cells.

117. The method of claim 107 or 108, wherein the mRNA is detected by RT-PCR.

118. The method of claim 107 or 108, wherein the nucleic acid sequence encoding the detectable mRNA comprises an intron.

119. The method of claim 118, wherein the detectable mRNA is expressed following mRNA processing in a mammalian cell.

120. The method of claim 107 or 108, wherein the method is a high throughput method and the cells are immobilized on an array.

121. A method of selecting internalizing ligand/anti-ligand pairs, comprising:
(a) contacting a library of ligand displaying genetic packages comprising a nucleic acid sequence encoding a detectable mRNA that is expressed upon internalization of the package with a cell(s) that expresses an anti-ligand-receptor fusion protein on its surface; and
(b) detecting the detectable mRNA expressed by the cell(s), and thereby selecting internalizing ligand/anti-ligand pairs.

122. A method of identifying an internalizing ligand and/or an anti-ligand of a ligand/anti-ligand pair, comprising the method of claim 121 and further comprising recovering a nucleic acid molecule encoding an internalizing ligand and/or a nucleic acid molecule encoding an internalizing anti-ligand from cell(s) expressing the detectable mRNA, and thereby identifying a ligand and/or anti-ligand of an internalizing ligand/anti-ligand pair.

123. The method of claim 121 or 122, wherein the library is contacted with the cell(s) in vivo.

124. The method of claim 121 or 122, wherein the library is contacted with the cell(s) for 24-96 hours prior to the detection.

125. The method of claim 121 or 122, wherein the library is contacted with the cell(s) for 48-72 hours prior to the detection.

126. The method of claim 121 or 122, wherein the library is contacted with the cell(s) for 72-96 hours prior to the detection.

127. The method of claim 121 or 122, wherein the library is contacted with the cell(s) for at least 96 hours prior to the detection.

128. The method of claim 121 or 122, wherein the mRNA is detected in substantially non-vasculature cells.

129. The method of claim 121 or 122, wherein the mRNA is detected in parenchymal cells.

130. The method of claim 121 or 122, wherein the mRNA is detected by RT-PCR.

131. The method of claim 121 or 122, wherein the nucleic acid sequence encoding the detectable mRNA comprises an intron.

132. The method of claim 131, wherein the detectable mRNA is expressed following mRNA processing in a mammalian cell.

133. The method of claim 121 or 122, wherein the method is a high throughput method and the cells are immobilized on an array.

134. A method of identifying a target cell or tissue for an internalizing ligand, comprising:
(a) contacting a ligand displaying genetic package comprising a detectable DNA with a cell(s) or a tissue(s); and
(b) detecting the detectable DNA in the cell(s) or tissue(s), and thereby identifying a target cell or tissue for internalizing ligands.

135. The method of claim 134, wherein the ligand displaying package is contacted with the cell(s) or tissue(s) in vivo.

136. The method of claim 134, wherein the ligand displaying genetic packages comprises an in-frame insert.

137. The method of claim 134, wherein the ligand displaying genetic package is contacted with the cell(s) or tissue(s) for 24-96 hours prior to the detection.

138. The method of claim 134, wherein the ligand displaying package is contacted with the (cell)s or tissue(s) for 48-72 hours prior to the detection.

139. The method of claim 134, wherein the ligand displaying package is contacted with the (cell)s or tissue(s) for 72-96 hours prior to the detection.

140. The method of claim 134, wherein the ligand displaying package is contacted with the (cell)s or tissue(s) for at least 96 hours prior to the detection.

141. The method of claim 134, wherein the DNA is detected in substantially non-vasculature cells.

142. The method of claim 134, wherein the DNA is detected in parenchymal cells.

143. The method of claim 134, wherein the DNA is detected by Hirt extraction.

144. The method of claim 134, wherein the DNA is detected by PCR.

145. The method of claim 134, wherein the DNA is detected by rolling circle amplification.

146. The method of claim 145, wherein the rolling circle amplification is performed using Phi 29 polymerase or exo(−)BST DNA polymerase.

147. The method of claim 134, wherein the DNA is detected by its ability to bind a specific DNA binding protein or nucleic acid.

148. The method of claim 134, wherein the method is a high throughput method and the cells are immobilized on an array.

149. A method of selecting an internalizing ligand displayed on a genetic package, comprising:
(a) contacting a library of ligand displaying genetic packages comprising a detectable DNA with a cell(s), and
(b) detecting the detectable DNA in the cell(s); and thereby selecting an internalizing ligand displayed on a genetic package.

150. A method of identifying an internalizing ligand displayed on a genetic package, comprising the method of claim 149 and further comprising recovering a nucleic acid molecule encoding the internalizing ligand from cells comprising the detectable DNA, and thereby identifying an internalizing ligand.

151. The method of claims 149 or 150, wherein the library is contacted with the cell(s) in vivo.

152. The method of claim 149 or 150, wherein the library is contacted with the cell(s) for 24-96 hours prior to the detection.

153. The method of claim 149 or 150, wherein the library is contacted with the cell(s) for 48-72 hours prior to the detection.

154. The method of claim 149 or 150, wherein the library is contacted with the cell(s) for 72-96 hours prior to the detection.

155. The method of claim 149 or 150, wherein the library is contacted with the cell(s) for at least 96 hours prior to the detection.

156. The method of claim 149 or 150, wherein the DNA is detected in substantially non-vasculature cells.

157. The method of claim 149 or 150, wherein the DNA is detected in parenchymal cells.

158. The method of claim 149 or 150, wherein the DNA is detected by Hirt extraction.

159. The method of claim 149 or 150, wherein the DNA is detected by PCR.

160. The method of claim 149 or 150, wherein the DNA is detected by rolling circle amplification.

161. The method of claim 160, wherein the rolling circle amplification is performed using Phi 29 polymerase or exo(−)BST DNA polymerase.

162. The method of claim 149 or 150, wherein the DNA is detected by its ability to bind a specific DNA binding protein or nucleic acid.

163. The method of claim 149 or 150, wherein the method is a high throughput method and the cells are immobilized on an array.

164. A method of selecting internalizing ligand/anti-ligand pairs, comprising:
(a) contacting a library of ligand displaying genetic packages comprising a detectable DNA with a cell(s) that expresses an anti-ligand-receptor fusion protein on its surface; and
(b) detecting the detectable DNA in the cell(s), and thereby selecting internalizing ligand/anti-ligand pairs.

165. A method of identifying an internalizing ligand and/or an anti-ligand of a ligand/anti-ligand pair, comprising the method of claim 164 and further comprising recovering a nucleic acid molecule encoding an internalizing ligand and/or a nucleic acid molecule encoding an internalizing anti-ligand from cell(s) comprising the detectable DNA, and thereby identifying a ligand and/or anti-ligand of an internalizing ligand/anti-ligand pair.

166. The method of claim 164 or 165, wherein the library is contacted with the cell(s) in vivo.

167. The method of claim 164 or 165, wherein the library is contacted with the cell(s) for 24-96 hours prior to the detection.

168. The method of claim 164 or 165, wherein the library is contacted with the cell(s) for 48-72 hours prior to the detection.

169. The method of claim 164 or 165, wherein the library is contacted with the cell(s) for 72-96 hours prior to the detection.

170. The method of claim 164 or 165, wherein the library is contacted with the cell(s) for at least 96 hours prior to the detection.

171. The method of claim 164 or 165, wherein the DNA is detected in substantially non-vasculature cells.

172. The method of claim 164 or 165, wherein the DNA is detected in parenchymal cells.

173. The method of claim 164 or 165, wherein the DNA is detected by Hirt extraction.

174. The method of claim 164 or 165, wherein the DNA is detected by PCR.

175. The method of claim 164 or 165, wherein the DNA is detected by rolling circle amplification.

176. The method of claim 175, wherein the rolling circle amplification is performed using Phi 29 polymerase or exo(−)BST DNA polymerase.

177. The method of claim 164 or 165, wherein the method is a high throughput method and the cells are immobilized on an array.

178. A method of identifying a target cell or tissue for an internalizing ligand, comprising:
(a) contacting a ligand displaying genetic packages comprising a nucleic acid sequence encoding a detectable product with a cell(s) or tissue(s), wherein the nucleic acid sequence comprises an intron; and
(b) detecting products expressed by the cell(s) or tissue(s), and thereby identifying a target cell or tissue for an internalizing ligand.

179. The method of claim 178, wherein the genetic package is contacted with the cell(s) or tissue(s) in vivo.

180. The method of claim 178, wherein the cell(s) or tissue(s) are contacted with a library of genetic packages comprising in-frame inserts.

181. The method of claim 178, wherein the genetic package is contacted with the cell(s) or tissue(s) for 24-96 hours prior to the detection.

182. The method of claim 178, wherein the genetic package is contacted with the (cell)s or tissue(s) for 48-72 hours prior to the detection.

183. The method of claim 178, wherein the genetic package is contacted with the (cell)s or tissue(s) for 72-96 hours prior to the detection.

184. The method of claim 178, wherein the genetic package is contacted with the (cell)s or tissue(s) for at least 96 hours prior to the detection.

185. The method of claim 178, wherein the product is detected in substantially non-vasculature cells.

186. The method of claim 178, wherein the product is detected in parenchymal cells.

187. The method of claim 178, wherein the product detected is a polypeptide.

188. The method of claim 187, wherein the polypeptide confers drug resistance to the cell(s) or tissue(s).

189. The method of claim 178, wherein the product detected is an mRNA.

190. The method of claim 187, wherein the mRNA is detected by RT-PCR.

191. The method of claim 178, wherein the detectable product is expressed following mRNA processing in a mammalian cell.

192. The method of claim 178, wherein the method is a high throughput method and the cells are immobilized on an array.

193. A method of selecting an internalizing ligand displayed on a genetic package, comprising:
(a) contacting a ligand displaying genetic package comprising a nucleic acid sequence encoding a detectable product with a cell(s), wherein the nucleic acid sequence comprises an intron; and
(b) detecting product expressed by the cell(s); and thereby selecting an internalizing ligand displayed on a genetic package.

194. A method of identifying an internalizing ligand displayed on a genetic package, comprising the method of claim 193 and further comprising recovering a nucleic acid molecule encoding the internalizing ligand from cell(s) expressing the detectable product, and thereby identifying an internalizing ligand.

195. The method of claim 193 or 194, wherein the genetic package is contacted with the cell(s) in vivo.

196. The method of claim 193 or 194, wherein the genetic package is contacted with the cell(s) for 24-96 hours prior to the detection.

197. The method of claim 193 or 194, wherein the genetic package is contacted with the cell(s) for 48-72 hours prior to the detection.

198. The method of claim 193 or 194, wherein the genetic package is contacted with the cell(s) for 72-96 hours prior to the detection.

199. The method of claim 193 or 194, wherein the genetic package is contacted with the cell(s) for at least 96 hours prior to the detection.

200. The method of claim 193 or 194, wherein the product is detected in substantially non-vasculature cells.

201. The method of claim 193 or 194, wherein the product is detected in parenchymal cells.

202. The method of claim 193 or 194, wherein the product detected is a polypeptide.

203. The method of claim 202, wherein the polypeptide confers drug resistance to the cell(s).

204. The method of claim 193 or 194, wherein the product detected is an mRNA.

205. The method of claim 204, wherein the mRNA is detected by RT-PCR.

206. The method of claim 193 or 194, wherein the detectable product is expressed following mRNA processing in a mammalian cell.

207. The method of claim 193 or 194, wherein the method is a high throughput method and the cells are immobilized on an array.

208. A method of selecting internalizing ligand/anti-ligand pairs, comprising:
(a) contacting a library of ligand displaying genetic packages comprising a nucleic acid sequence encoding a detectable product with a cell(s), wherein the nucleic acid sequence comprises an intron, and wherein the cell(s) expresses an anti-ligand-receptor fusion protein on its surface; and
(b) detecting product expressed by the cell(s), and thereby selecting internalizing ligand/anti-ligand pairs.

209. A method of identifying an internalizing ligand and/or an anti-ligand of a ligand/anti-ligand pair, comprising the method of claim 209 and further comprising recovering a nucleic acid molecule encoding an internalizing ligand and/or a nucleic acid molecule encoding an internalizing anti-ligand from cell(s) that express the detectable product, and thereby identifying a ligand and/or anti-ligand of an internalizing ligand/anti-ligand pair.

210. The method of claim 208 or 209, wherein the library is contacted with the cell(s) in vivo.

211. The method of claim 208 or 209, wherein the library is contacted with the cell(s) for 24-96 hours prior to the detection.

212. The method of claim 208 or 209, wherein the library is contacted with the cell(s) for 48-72 hours prior to the detection.

213. The method of claim 208 or 209, wherein the library is contacted with the cell(s) for 72-96 hours prior to the detection.

214. The method of claim 208 or 209, wherein the library is contacted with the cell(s) for at least 96 hours prior to the detection.

215. The method of claim 208 or 209, wherein the product is detected in substantially non-vasculature cells.

216. The method of claim 208 or 209, wherein the product is detected in parenchymal cells.

217. The method of claim 208 or 209, wherein the product detected is a polypeptide.

218. The method of claim 217, wherein the polypeptide confers drug resistance to the cell(s).

219. The method of claim 208 or 209, wherein the product detected is an mRNA.

220. The method of claim 219, wherein the mRNA is detected by RT-PCR.

221. The method of claim 208 or 209, wherein the detectable product is expressed following mRNA processing in a mammalian cell.

222. The method of claim 208 or 209, wherein the method is a high throughput method and the cells are immobilized on an array.
